(19) United States
(12) Patent Application Publication
(10) Pub. No.: US 2026/0327318 A1
(43) Pub. Date: Oct. 22, 2026

(54) FGFR3 S249C X CD3 BISPECIFIC ANTIBODIES AND METHODS OF USE

(71) Applicant: Merus B.V., Utrecht (NL)

(72) Inventors: Colleen Ann BURNS, New York, NY (US); Kyla Elizabeth DRISCOLL, Rutherford, NJ (US); Omar DURAMAD, Berkeley, CA (US); Rikke B. HOLMGAARD, West New York, NJ (US); Kevin LINDQUIST, Berkeley, CA (US); Joshua PARK, Castro Valley, CA (US); Mohan SRINIVASAN, Cupertino, CA (US); Xuan ZHAO, South San Francisco, CA (US); Suzanne Marleen BAL, Utrecht (NL); Nesrine CHAKROUN, Utrecht (NL); Cornelis Adriaan DE KRUIF, Utrecht (NL); Rinse KLOOSTER, Utrecht (NL); Pieter Fokko VAN LOO, Utrecht (NL); Martijn VAN ROSMALEN, Utrecht (NL); Jaina M. PATEL, New York, NY (US)

(21) Appl. No.: 19/543,669

(22) Filed: Feb. 18, 2026

Related U.S. Application Data
(60) Provisional application No. 63/760,411, filed on Feb. 19, 2025.

(51) Int. Cl. C07K 16/28; A61K 39/00; A61K 47/68; A61P 35/00

(52) U.S. Cl. CPC C07K 16/2863; A61K 47/6849; A61P 35/00; C07K 16/2809; A61K 2039/505; C07K 2317/10; C07K 2317/31

(57) ABSTRACT

The present invention relates to antibodies that specifically bind human FGFR3IIIb S249C, to bispecific antibodies that specifically bind to both human FGFR3IIIb S249C and human CD3, compositions comprising such antibodies, and methods of using the same.

### CROSS-REFERENCE TO RELATED APPLICATIONS

This application claims priority to U.S. Provisional Application No. 63/760,411 filed Feb. 19, 2025.

### REFERENCE TO A SEQUENCE LISTING SUBMITTED ELECTRONICALLY VIA EFS WEB

This application includes a Sequence Listing submitted electronically via EFS-web (Name 4096_0690001_SequenceListing_ST26.xml, File Size: 47,173 bytes, and Created On Mar. 10, 2026), which is hereby incorporated by reference in its entirety.

### FIELD OF THE INVENTION

The present disclosure relates to antibodies that specifically bind a variant of the human fibroblast growth factor receptor 3 (FGFR3) carrying the serine to cysteine mutation at position 249 (S249C), to bispecific antibodies that specifically bind both human FGFR3IIIb S249C and human CD3, compositions comprising such antibodies, and methods of using such antibodies for the treatment of certain cancers and other diseases.

### BACKGROUND

The American Association for Cancer Research (AACR) Project GENIE® is a publicly accessible cancer registry of real-world clinico-genomic data assembled through data sharing between 19 leading international cancer centers. The registry aggregates, harmonizes, and links clinical-grade, next-generation cancer genomic sequencing data with clinical outcomes obtained during routine medical practice from nearly every cancer patient treated at participating institutions. The FGFR3 S249C gene mutation is found in approximately 0.43% of such AACR Project GENIE® cases, including being a hotspot mutation for bladder cancer, where approximately 15% of muscle invasive bladder cancers (MIBC) and up to 60% of non-MIBC harbor this mutation. As a result, targeting FGFR3 S249C may be an important therapeutic option across multiple tumor types.

In the treatment of bladder cancer, cisplatin has been the cornerstone of all therapeutic strategies for more than forty years. However, only about 50% of patients are eligible for cisplatin-based treatments (Galsky M D, J Clin Oncol 2011; 29:2432-8). Moreover, the survival benefits are relatively modest, with a median overall survival of 13-15 months, with approximately 15% long responses (Von der Maase H, J Clin Oncol 2005; 23:4602-8). More recently, the pan-FGFR tyrosine kinase inhibitor, Balversa® or erdafitinib, was approved in 2019 to treat metastatic or locally advanced bladder cancer harboring FGFR2 and/or FGFR3 gene fusions or one or more FGFR3 mutations, including the S249C mutation (Loriot, Y., et al., NEJM Oct. 20, 2023; 389:1961-71). However with erdafitinib the blockade of multiple FGFR family members led to several types of toxicity, including gastrointestinal, nail, skin and ocular toxicity, as well as hyperphosphatemia (Yang et al., Cancer Research 2024). Thus, finding ways to specifically target the S249C mutation on FGFR3 should mitigate at least some of these toxicity findings and thereby enhance the therapeutic safety profile.

Another issue with current therapies is when 70-80% of NMIBC patients develop at least one recurrence within 5 years after the initial treatment, and 10-20% of these patients will progress into MIBC (Sikic, D. et al., Cancer Manag. Res. 13, 6567-6578 (2021)). In addition, the recurrence and metastasis rates for MIBC patients are around 50%, with the 5-year overall survival less than 50% (Giannopoulou, A. F. et al., Int. J. Mol. Sci. 20 (2019)). This may be due to an observed acquired drug resistance where “gatekeeper” mutations emerge, which have been shown to block access to a portion of the binding pocket accessed by current pan-FGFR inhibitors. See Krook, M. A., et al., British J Cancer, 2021; 124:880-892. Thus, durable responses are still uncommon, and the majority of patients relapse and succumb to metastatic disease.

Antibodies targeting FGFR3 and its mutations are known in the art-see for example, without limitation, WO2010/111367, WO2015/094900, WO2016/134234, WO2021/010326, and WO2024/107759. Some of these molecules have been tested in human clinical trials, but as of yet no FGFR3 antibody therapeutic has been approved.

Therefore, there is a need to identify alternative therapies to successfully treat cancers involving the FGFR3 S249C mutation, particularly if patients relapse while on currently approved therapies, and at the same time mitigate any cytokine release syndrome (CRS), immune effector cell-associated neurotoxicity syndrome (ICANS), also referred to as neurotoxicity, and other toxicities. The present disclosure provides an advance in the art by providing human FGFR3IIIb S249C× human CD3 bispecific antibodies of the invention, compositions comprising such bispecific antibodies, and methods useful in the treatment of certain cancers by using such bispecific antibodies.

### DESCRIPTION OF THE FIGURES

FIG. 1 shows a schematic of human FGFR3 structure, indicating the location of the S249C mutation in the extracellular portion of the molecule.

FIG. 2 shows in vitro cell binding of TCEs of the invention to human CD3 expressing Jurkat/NFAT cells using TCE samples collected on a flow cytometer. Data is shown as % Activity. See Example 2.

FIGS. 3A-B show in vitro cell binding of TCEs of the invention at 15, 5 or 1 μg/ml concentrations to isolated human T cells from two human donors (RG4063 and RG3387) using TCE samples collected on a flow cytometer. Data was plotted on GraphPad Prism and shown as the MFI Geo mean with standard deviation. See Example 2.

FIGS. 4A-E show the in vitro binding specificity of TCEs of the invention to engineered HeLa cells expressing the S249C mutant FGFR3 isoform IIIb (FIG. 4A-D) or to engineered HeLa cells expressing the S248C mutant FGFR3 isoform IIIb (FIG. 4E). Values for % activity were plotted in GraphPad Prism version 10.1.2. See Example 3.

FIGS. 5A-D show the in vitro binding of TCEs of the invention to engineered Hela cells expressing the wild-type (WT) FGFR3 isoform IIIb. Values for % activity were plotted in GraphPad Prism version 10.1.2. See Example 3.

FIGS. 6A-C show the in vitro binding of TCEs of the invention to engineered Hela cells expressing the S249C mutant FGFR3 isoform IIIc. Values for % activity were plotted in GraphPad Prism version 10.1.2. See Example 3.

FIGS. 7A-C show the in vitro binding of TCEs of the invention to engineered HeLa cells expressing the wild-type (WT) FGFR3 isoform IIIc. Values for % activity were plotted in GraphPad Prism version 10.1.2. See Example 3.

FIGS. 8A-C show the in vitro binding activity of TCEs of the invention to normal primary bladder epithelial cells (BdEC), which do not express the S249C mutation. Data were analyzed in FlowJo and plotted in GraphPad Prism as a mean+/−standard deviation. See Example 4.

FIGS. 9A-C show the in vitro binding activity of TCEs of the invention to RT112 bladder cancer cells expressing a FGFR3-TACC3 fusion, which does not express the S249C mutation. Data were analyzed in FlowJo® and values plotted in GraphPad Prism as a mean+/−standard deviation. See Example 5.

FIGS. 10A-B show cell binding by flow cytometry of TCEs of the invention on DEKK backbones to HEK293T cells engineered to express FGFR1 isoform IIIb (FIG. 10A) and FGFR1 isoform IIIc (FIG. 10B). Data was plotted on GraphPad Prism, shown as a mean with standard deviation. See Example 6.

FIGS. 11A-B show cell binding by flow cytometry of TCEs of the invention on DEKK backbones to HEK293T cells engineered to express FGFR2 isoform IIIb (FIG. 11A) and FGFR2 isoform IIIc (FIG. 11B). Data was plotted on GraphPad Prism, shown as a mean with standard deviation. See Example 6.

FIGS. 12A-D show cell binding by flow cytometry of TCEs of the invention on DEKK backbones to HEK293T cells engineered to express FGFR3 isoform IIIb wild-type (FIG. 12A), FGFR1 isoform IIIb S249C (FIG. 12C), FGFR3 isoform IIIc wild-type (FIG. 12B), and FGFR3 isoform IIIc S249C (FIG. 12D). Data was plotted on GraphPad Prism, shown as a mean with standard deviation. See Example 6.

FIG. 13 shows cell binding by flow cytometry of TCEs of the invention on DEKK backbones to HEK293T cells engineered to express FGFR4. Data was plotted on GraphPad Prism, shown as a mean with standard deviation. See Example 6.

FIG. 14 shows the in vitro binding activity of TCEs of the invention to human CD3 heterodimer epsilon delta-His recombinant protein as assessed by ELISA. See Example 7.

FIG. 15 shows the in vitro binding activity of TCEs of the invention to human CD3 heterodimer epsilon gamma-His recombinant protein as assessed by ELISA. See Example 7.

FIGS. 16A-B show the in vitro binding activity of TCEs of the invention to recombinant FGFR3IIIb S249C FIG. 16A) and to recombinant wild-type (WT) FGFR3 isoform IIIb (FIG. 16B) as assessed by ELISA. See Example 8.

FIGS. 17A-B show the in vitro binding activity of TCEs of the invention to recombinant FGFR3IIIc S249C (FIG. 17A) and to recombinant wild-type (WT) FGFR3 isoform IIIc (FIG. 17B) as assessed by ELISA. See Example 8.

FIGS. 18A-B show the ex vivo cytotoxic activity of TCEs of the invention to FGFR3 S249C positive SCBO1 organoid model co-cultured with PBMCs obtained from two different human donors (Donor CC01319-FIG. 18A and Donor CC01344-FIG. 18B) as assessed by luminescence measured by a microplate spectrophotometer as a % viability. See Example 9 and Table 6.

FIGS. 19A-B show the ex vivo cytotoxic activity of TCEs of the invention to FGFR3 S249C positive SCBO1 organoid model co-cultured with PBMCs obtained from two different human donors (Donor CC01319-FIG. 19A and Donor CC01344-FIG. 19B) as assessed by luminescence measured by a microplate spectrophotometer as a % viability. See Example 9 and Table 4.

FIGS. 20A-C show the ex vivo cytotoxic activity of TCEs of the invention to wild-type (WT) FGFR3 SCBO1 organoid model co-cultured with PBMCs obtained from two different human donors (Donor CC01319-FIG. 20A and Donor CC01344-FIG. 20B) or without any human donor PBMCs as a control (FIG. 20C) as assessed by luminescence measured by a microplate spectrophotometer as a % viability. See Example 9 and Table 5.

FIGS. 21A-D show the in vitro cytotoxicity specificity of TCEs of the invention against FGFR3IIIb S249C expressing HeLa cells (FIG. 21A-B) as compared to controls (FIG. 21C-D). See Example 10.

FIGS. 22A-D show the in vitro cytotoxicity specificity of TCEs of the invention against FGFR3IIIb wild-type (WT) expressing HeLa cells (FIG. 22A-B) as compared to controls (FIG. 22C-D). See Example 10.

FIGS. 23A-D show the in vitro cytotoxicity specificity of TCEs of the invention against HeLa parental cells (FIG. 23A-B) as compared to controls (FIG. 23C-D). See Example 10.

FIGS. 24A-D show the in vitro cytotoxicity specificity of TCEs of the invention against a bladder cancer cell line UMUC-14 that endogenously express FGFR3IIIb S249C using human PBMCs-Donor 11423 (FIG. 24A-B) as compared to controls (FIG. 24C-D). See Example 11.

FIGS. 25A-D show the in vitro cytotoxicity of TCEs of the invention against a bladder cancer cell line UMUC-14 using human PBMCs-Donor 13086 (FIG. 25A-B) as compared to controls (FIG. 25C-D). See Example 11.

FIGS. 26A-C show the in vitro cytotoxicity of TCEs of the invention against a bladder cancer cell line UMUC-14 using human PBMCs-Donor CC01319 (FIG. 26A) as compared to controls (FIG. 26B-C). See Example 11.

FIGS. 27A-D show the in vitro cytotoxicity specificity of TCEs of the invention against an FGFR3IIIb wild-type expressing human cancer cell line, HepG2 in the presence of a human donor PBMC in a 10:1 effector-to-target cell ratio (FIG. 27A-B) as compared to controls (FIG. 26C-D). Percent lysis was calculated using CellTiter-Glo® luminescent cell viability assay. See Example 12.

FIGS. 28A-C show the in vitro cytotoxicity specificity of TCEs of the invention against an FGFR3IIIb wild-type expressing human cancer cell line, HepG2 in the presence of a human donor PBMC in a 10:1 effector-to-target cell ratio (FIG. 28A-B) as compared to controls (FIG. 26B-C). Percent lysis was calculated using CellTiter-Glo® luminescent cell viability assay. See Example 12.

FIGS. 29A-D show the in vitro cytotoxicity specificity of TCEs of the invention against an FGFR3 S249C expressing human cancer cell line, OMC-1 in the presence of expanded human T cells in a 10:1 effector-to-target cell ratio (FIG. 29A-B) as compared to controls (FIG. 29C-D). Percent lysis was calculated using CellTiter-Glo Luminescent cell viability assay. See Example 13.

FIG. 30 shows the anti-tumor activity of TCEs of the invention in a transgenic mouse model utilizing homozygous human CD3 epsilon delta gamma knock-in mice implanted with murine breast cancer EO771-huFGFR1IIb S249C cells at a 0.44 mg/kg dose. See Example 14.

FIG. 31 shows the effect of TCEs of the invention on body weight in the E0771-huFGFR1IIb S249C cells transgenic mouse model. See Example 14.

FIGS. 32A-B show the anti-tumor activity of TCEs of the invention in a transgenic mouse model utilizing homozygous human CD3 epsilon delta gamma knock-in mice implanted with murine breast cancer E0771-huFGFR1IIb WT cells at a 0.44, 0.15, 0.05, and 0.017 mg/kg dose (FIG. 32A). The effect of TCEs of the invention on body weight in the E0771-huFGFR1IIb WT cells transgenic mouse model is shown in FIG. 32B. See Example 14.

FIGS. 33A-B show the anti-tumor effect of TCEs of the invention in a preventative murine model in which HeLa-FGFR3IIIb S249C overexpressing cell line and human PBMCs were co-implanted (FIG. 33A). Tumor volume changes from baseline were measured as a tumor growth inhibition percentage.

FIG. 33B shows the effect of TCEs of the invention on body weight in the preventative HeLa-FGFR3IIIb S249C co-implantation mouse model. See Example 15.

FIG. 34 shows the anti-tumor effect of 0.44 mg/kg of TCEs of the invention in a humanized UMUC-14 murine xenograft model in which human PBMCs were co-implanted. Tumor volume changes from baseline were measured as a tumor growth inhibition percentage. See Example 16.

FIG. 35 shows the effect of 0.44 mg/kg of TCEs of the invention on body weight in the humanized UMUC-14 murine xenograft model co-implantation mouse model. See Example 16.

FIGS. 36A-B show the treatment effect of various doses of TCEs of the invention on tumor growth in a UMUC-14 xenograft mouse model co-implanted with human PBMCs (FIG. 36A). Tumor volumes are shown as a geometric mean±SEM, n=6 per group. FIG. 36B shows the effect of TCEs of the invention on body weight in the same xenograft mouse model. Body weight was measured bi-weekly. The data are presented as least squares mean±SEM (n=6) and are plotted for each treatment group versus time. See Example 16.

FIGS. 37A-B show the treatment effect of various doses of TCEs of the invention on tumor growth in a RT-112 xenograft mouse model co-implanted with human PBMCs (FIG. 37A). Tumor volumes are shown as a geometric mean±SEM, n=5 per group. FIG. 37B shows the effect of TCEs of the invention on body weight in the same xenograft mouse model. Body weight was measured bi-weekly. The data are presented as least squares mean±SEM (n=5) and are plotted for each treatment group versus time. See Example 16.

FIGS. 38A-B show analysis of cytokine release as a result of blood samples collected from the UMUC-14 xenograft study described in Example 16, where human IFNγ (FIG. 38A) and human IL-2 (FIG. 38B) were both quantified in pg/mL. See Example 17.

FIG. 39 shows the in vitro cytotoxicity of TCEs of the invention against a bladder cancer cell line UMUC-14 using human PBMCs as compared to controls. See Example 11.

FIGS. 40A-F show cytokine production of TCEs of the invention and controls after in vitro cytotoxicity activity in a bladder cancer cell line UMUC-14 using human PBMCs. See Example 11.

### SUMMARY OF THE INVENTION

The present invention describes bispecific T cell engager (TCE) antibodies for the treatment of various cancers, in particular those that are FGFR3 isoform IIIb S249C positive cancers, where one arm of the TCE specifically binds a human immune effector cell, human CD3, while the other arm specifically binds the human FGFR3 isoform IIIb carrying the S249C mutation. See for example, without limitation, Examples 3-8.

Compared with monoclonal antibodies, bispecific antibodies offer some advantages such as superior cytotoxic effects on cancers as there is a lower rate of resistance due to the ability to target two different antigens. Bispecific antibodies that are immune-engaging such as those that target the general marker of T cells cluster of differentiation 3 (CD3) on one arm and a tumor-specific antigen on the other arm are known as T cell engagers (TCE). When both T cell and tumor cell are bound by a TCE a cytolytic synapse is formed whereby the T cell is activated and releases the pore-forming perforin and cytotoxic granzyme-B, leading to killing of the targeted tumor cell.

TCEs can be divided into two categories: the immunoglobulin G (IgG)-based antibodies, such as Lunsumio®-mosunetuzumab, and the variable fragment (Fv)-based bispecifics such as Blincyto®-blinatumomab. Bispecifics based on the IgG structure display a similar structure to native antibodies. In general, compared with Fv-based bispecifics, IgG-based bispecifics have longer half-lives in vivo because they are larger in size so clearance by the kidney is more difficult. The solubility and stability of IgG-based bispecifics are also improved for the presence of the fragment crystallizable (Fc) domains over Fv-based bispecifics. As defined herein, recitation of the term TCEs will refer to IgG-based TCEs unless otherwise indicated.

Early methods of producing IgG-based TCEs involved combining half-molecules from heterogenous parental antibodies. However, such techniques were fraught with homo-dimer mispairings, for example co-expressing two heavy chains and two light chains to generate an IgG TCE can result in some mis-assembly and unwanted byproducts (Lewis S M et al., Nature Biotechnology 2014; 32:191-202; Leaver-Fay A, et al., Structure 2016; 24:641-651). Subsequent techniques of recombining functional half-molecules to produce IgG-based TCEs include, but are not limited to orthogonal Fab interface (Lewis, S. et al., Nature Biotech, 2014; 32:191-198), DuoBody®-Genmab, XmAb®-Xencor, CrossMab (Schaefer, W. et al., PNAS 2011; 108:11187-92), knobs-into-holes (KiH) (Ridgway, J. et al., Protein Eng. 1996; 9:617-21 and Atwell, S. et al., J Mol Biol 1997; 270:26-35), and Biclonics® (De Nardis, C. et al., JBC 2017; 292 (35): 14706-14717), all of which increase the likelihood of proper heterodimerization.

Another challenge is that TCEs require the correct pairing of two distinct light chains. Random light chain association would otherwise lead to a mixture of species with correctly assembled antibodies occurring only 25% of the time. Since the specific binding of antibodies to an antigen is mainly provided by the heavy chain, such specific antigen binding can be preserved when such an antibody has a non-cognate light chain. Use of a common light chain (cLC) combined with two different heavy chains avoids Fab mispairings. Thus, only three peptide chains need to be expressed, rather than four, which is a clear advantage in terms of manufacturability as well as simplifying the purification process for typical biologics production. Common light chain antibodies have been isolated from phage scFv display libraries with a restricted light chain diversity (Merchant et al., Nature Biotech, 1998, 16:677-681) or phage Fab libraries with a unique light chain (Jackman et al., J Biol Chem 2010 Jul. 2; 285 (27)) or obtained by immunizing transgenic mice expressing a cLC.

Thus, the present disclosure provides human FGFR3IIIb S249C× human CD3 bispecific TCEs that exhibit in vivo efficacy by specifically binding to the mutant S249C form of FGFR3IIIb while minimizing CRS in at least one pre-clinical model of an FGFR3 isoform IIIb S249C positive cancer.

Cluster of differentiation 3 (CD3) is a protein complex and a T cell co-receptor that is involved in activating both the cytotoxic T cell (CD8+ naive T cells) and T helper cells (CD4+ naive T cells). It is composed of four distinct chains. In mammals, the complex contains a CD3γ chain (SwissProt P09693), a CD38 chain (SwissProt P04234), and two CD38 chains (SwissProt P07766). These chains associate with the T-cell receptor (TCR) and the CD3-zeta (ξ-chain) to generate an activation signal in T lymphocytes. The TCR, CD3-zeta, and the other CD3 molecules together constitute the TCR complex.

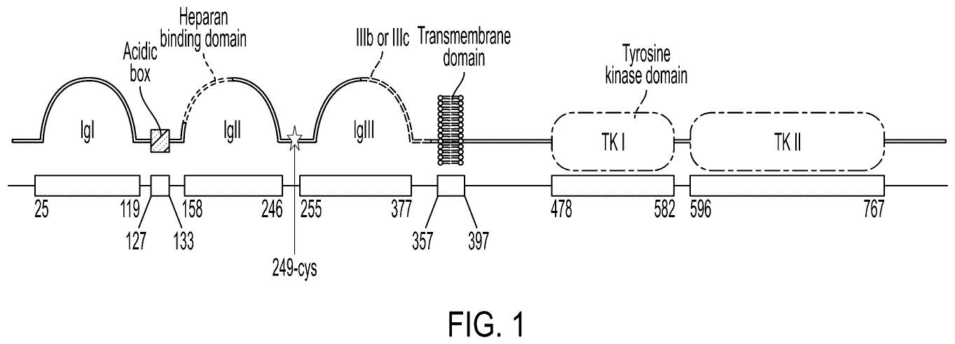

Fibroblast growth factor receptor 3 (FGFR3) is a member of the fibroblast growth factor receptor tyrosine kinase family which interact with various fibroblast growth factors. Five distinct membrane FGFRs have been identified in vertebrates, all belonging to the tyrosine kinase super family: FGFR1-4, 6 and FGFR-like 1 (FGFRL1). Several isoforms of the FGFR3 protein exist and are found in various tissues of the body, and they interact with a variety of growth factors. These proteins play a role in several important cellular processes, including regulation of cell growth and division (proliferation), determination of cell type, formation of blood vessels (angiogenesis), wound healing, and embryo development. As depicted in FIG. 1, FGFR3 consists of three domains: an extracellular ligand-binding domain (ECD), a single transmembrane domain (TM), and an intracellular tyrosine kinase domain. The ECD comprises a hydrophobic signal peptide region and three immunoglobulin (Ig)-like domains D1-D3. The TM facilitates signal transduction from the extracellular region into the cytoplasmic domain. Following the cytoplasmic membrane is a juxtamembrane region, then two tyrosine kinase domains TK1 and TK2 and then a carboxyl tail COOH. An array of isoforms exists due to alternative splicing of endogenous mRNA sequences, mainly of the IgIII domain (D3), to form either an FGFR3 IIIb isoform or an FGFR3 IIIc isoform, see FIG. 1. Another isoform includes a secreted form lacking the transmembrane domain and entire cytoplasmic domain. Under normal wild-type FGFR3 conditions, different isoforms are found in different tissues, for example the normal wild-type IIIb isoform of FGFR3 is the main form in epithelial cells and thus found in skin, corneal cells of the eye and in the esophagus. The normal wild-type IIIc isoform of FGFR3 is the main form in mesenchyme-derived cells and can be found, for example, in the brain and testis. The S249C mutation is found in the extracellular domain between the Ig-like domains IgII D2 and IgIII D3 of FGFR3 (see FIG. 1) and is known as an activating mutation because it introduces an additional unpaired cysteine and leads to constitutive phosphorylation of the receptor independent of FGF ligand stimulation. Tomlinson, D. et al., Oncogene 2007 Aug. 30; 26 (40): 5889-5899. While both isoforms IIIb and IIIc can carry the S249C mutation and be linked to certain diseases, certain cancers can show elevated expression of one isoform mutation over the other, for example without limitation, the isoform IIIb carrying the S249C mutation is predominantly expressed in bladder cancer and other urothelial cell carcinomas.

### EMBODIMENTS

Embodiments of the present invention are contemplated to include, but are not limited to the following:
1. A bispecific antibody that specifically binds human fibroblast growth factor receptor 3 isoform IIIb serine 249 cysteine (hFGFR3IIIb S249C) and human CD3 (hCD3), wherein the bispecific antibody comprises (a) a first antigen binding domain that specifically binds hFGFR3IIIb S249C comprising a first heavy chain variable region (VH1) and a light chain variable region (VL1), wherein the VH1 comprises heavy chain complementarity determining regions (HCDR) HCDR1, HCDR2, and HCDR3, and the VL1 comprises light chain complementarity determining regions (LCDR) LCDR1, LCDR2, and LCDR3, wherein: (a) HCDR1 comprises SEQ ID NO: 1, HCDR2 comprises SEQ ID NO:2, HCDR3 comprises SEQ ID NO: 3, LCDR1 comprises SEQ ID NO:4, LCDR2 comprises SEQ ID NO:5, LCDR3 comprises SEQ ID NO: 6; (b) HCDR1 comprises SEQ ID NO:1, HCDR2 comprises SEQ ID NO:7, HCDR3 comprises SEQ ID NO: 8, LCDR1 comprises SEQ ID NO:4, LCDR2 comprises SEQ ID NO:5, LCDR3 comprises SEQ ID NO: 6; (c) HCDR1 comprises SEQ ID NO:9, HCDR2 comprises SEQ ID NO: 10, HCDR3 comprises SEQ ID NO: 11, LCDR1 comprises SEQ ID NO:4, LCDR2 comprises SEQ ID NO:5, LCDR3 comprises SEQ ID NO: 6; or (d) HCDR1 comprises SEQ ID NO:1, HCDR2 comprises SEQ ID NO:7, HCDR3 comprises SEQ ID NO: 12, LCDR1 comprises SEQ ID NO:4, LCDR2 comprises SEQ ID NO:5, LCDR3 comprises SEQ ID NO:6, and a second antigen binding domain that specifically binds human CD3. See Table 2.
2. The bispecific antibody of embodiment 1, wherein the second antigen binding domain that specifically binds hCD3 comprises a second heavy chain variable region (VH2) and a light chain variable region (VL2), wherein the VH2 comprises heavy chain complementarity determining regions (HCDR) HCDR4, HCDR5, and HCDR6, and the VL2 comprises light chain complementarity determining regions (LCDR) LCDR4, LCDR5, and LCDR6. See Table 2.
3. A bispecific antibody that specifically binds hFGFR3IIIb S249C and hCD3, wherein the bispecific antibody comprises (a) a first antigen binding domain that specifically binds hFGFR3IIIb S249C and (b) a second antigen binding domain that specifically binds hCD3 comprising a heavy chain variable region (VH2) and a light chain variable region (VL2), wherein the VH2 comprises heavy chain complementarity determining regions (HCDR) HCDR4, HCDR5, and HCDR6, and the VL2 comprises light chain complementarity determining regions (LCDR) LCDR4, LCDR5, and LCDR6, wherein: (a) HCDR4 comprises SEQ ID NO: 13, HCDR5 comprises SEQ ID NO: 14, HCDR6 comprises SEQ ID NO: 15, LCDR4 comprises SEQ ID NO:4, LCDR5 comprises SEQ ID NO:5, LCDR6 comprises SEQ ID NO:6; (b) HCDR4 comprises SEQ ID NO:16, HCDR5 comprises SEQ ID NO:17, HCDR6 comprises SEQ ID NO: 18, LCDR4 comprises SEQ ID NO:4, LCDR5 comprises SEQ ID NO:5, LCDR6 comprises SEQ ID NO: 6; or (c) HCDR4 comprises SEQ ID NO:19, HCDR5 comprises SEQ ID NO:20, HCDR6 comprises SEQ ID NO:21, LCDR4 comprises SEQ ID NO:4, LCDR5 comprises SEQ ID NO:5, LCDR6 comprises SEQ ID NO:6. See Table 2.
4. The bispecific antibody of any one of embodiments 1-3, wherein the bispecific antibody does not specifically bind to wild-type (WT) FGFR3, as measured by, for example, ELISA.
5. The bispecific antibody of any one of embodiments 1-4, wherein the bispecific antibody does not specifically bind to FGFR1, or FGFR2, or FGFR4, as measured by, for example, ELISA.
6. The bispecific antibody of any one of embodiments 1-5, wherein the bispecific antibody does not specifically bind to FGFR3 having the R248C mutation, as measured by, for example, ELISA.
7. The bispecific antibody of any one of embodiments 1-6, wherein the bispecific does not specifically bind to either wild-type (WT) or S249C mutant form of the FGFR3IIIc isoform, as measured by, for example, ELISA.
8. The bispecific antibody of any one of embodiments 1-7, wherein the first antigen binding domain has about an 0.5-fold to 10-fold greater binding affinity (K_{D}), or about a 1.5-fold to 10-fold greater binding affinity (K_{D}), or about a 2-fold to 5-fold greater binding affinity (K_{D}), or about a 2-fold to 4-fold greater binding affinity (K_{D}), or about a 2-fold to 3-fold greater binding affinity (K_{D}), or about a 2-fold greater binding affinity (K_{D}), or about a 3-fold greater binding affinity (K_{D}), or about a 4-fold greater binding affinity (K_{D}), or about a 5-fold greater binding affinity (K_{D}), or about a 6-fold greater binding affinity (K_{D}), or about a 7-fold greater binding affinity (K_{D}), or about a 8-fold greater binding affinity (K_{D}), or about a 9-fold greater binding affinity (K_{D}), or about a 10-fold greater binding affinity for hFGFR3IIIb S249C relative to the binding affinity the second antigen binding domain has for hCD3, as measured by, for example, surface plasmon resonance (SPR).
9. The bispecific antibody of any one of embodiments 1-8, wherein the bispecific antibody specifically binds to FGFR3IIIb S249C extracellular domain dimers, as measured by, for example, ELISA.
10. The bispecific antibody of embodiment 9, wherein the bispecific antibody does not bind to FGFR3IIIb S249C monomers, as measured by, for example, ELISA.
11. The bispecific antibody of any one of embodiments 1-10, wherein the bispecific does not prevent FGFR3 dimerization, as measured by, for example, ELISA.
12. The bispecific antibody of any one of embodiments 1-11, wherein the bispecific can be blocked by FGF ligand, as measured by, for example, ELISA.
13. The bispecific antibody of any one of embodiments 1-12, wherein the bispecific is not blocked by soluble FGFR3, as measured by, for example, ELISA.
14. The bispecific antibody of any one of embodiments 1-13, wherein the bispecific does not bind soluble FGFR3, as measured by, for example, ELISA.
15. The bispecific antibody of embodiment 2, wherein: (a) HCDR1 comprises SEQ ID NO:1, HCDR2 comprises SEQ ID NO: 2, HCDR3 comprises SEQ ID NO:3, LCDR1 comprises SEQ ID NO:4, LCDR2 comprises SEQ ID NO:5, LCDR3 comprises SEQ ID NO:6, HCDR4 comprises SEQ ID NO: 13, HCDR5 comprises SEQ ID NO:14, HCDR6 comprises SEQ ID NO: 15, LCDR4 comprises SEQ ID NO:4, LCDR5 comprises SEQ ID NO:5, LCDR6 comprises SEQ ID NO:6; (b) HCDR1 comprises SEQ ID NO: 1, HCDR2 comprises SEQ ID NO: 2, HCDR3 comprises SEQ ID NO:3, LCDR1 comprises SEQ ID NO: 4, LCDR2 comprises SEQ ID NO:5, LCDR3 comprises SEQ ID NO:6, HCDR4 comprises SEQ ID NO: 16, HCDR5 comprises SEQ ID NO:17, HCDR6 comprises SEQ ID NO: 18, LCDR4 comprises SEQ ID NO: 4, LCDR5 comprises SEQ ID NO:5, LCDR6 comprises SEQ ID NO:6; (c) HCDR1 comprises SEQ ID NO: 1, HCDR2 comprises SEQ ID NO: 7, HCDR3 comprises SEQ ID NO:8, LCDR1 comprises SEQ ID NO: 4, LCDR2 comprises SEQ ID NO:5, LCDR3 comprises SEQ ID NO:6, HCDR4 comprises SEQ ID NO: 19, HCDR5 comprises SEQ ID NO:20, HCDR6 comprises SEQ ID NO:21, LCDR4 comprises SEQ ID NO: 4, LCDR5 comprises SEQ ID NO:5, LCDR6 comprises SEQ ID NO:6; (d) HCDR1 comprises SEQ ID NO: 9, HCDR2 comprises SEQ ID NO: 10, HCDR3 comprises SEQ ID NO: 11, LCDR1 comprises SEQ ID NO: 4, LCDR2 comprises SEQ ID NO:5, LCDR3 comprises SEQ ID NO:6, HCDR4 comprises SEQ ID NO: 16, HCDR5 comprises SEQ ID NO: 17, HCDR6 comprises SEQ ID NO: 18, LCDR4 comprises SEQ ID NO: 4, LCDR5 comprises SEQ ID NO:5, LCDR6 comprises SEQ ID NO:6; (e) HCDR1 comprises SEQ ID NO: 1, HCDR2 comprises SEQ ID NO: 2, HCDR3 comprises SEQ ID NO:3, LCDR1 comprises SEQ ID NO: 4, LCDR2 comprises SEQ ID NO:5, LCDR3 comprises SEQ ID NO:6, HCDR4 comprises SEQ ID NO: 19, HCDR5 comprises SEQ ID NO:20, HCDR6 comprises SEQ ID NO:21, LCDR4 comprises SEQ ID NO: 4, LCDR5 comprises SEQ ID NO:5, LCDR6 comprises SEQ ID NO:6; (f) HCDR1 comprises SEQ ID NO: 9, HCDR2 comprises SEQ ID NO: 10, HCDR3 comprises SEQ ID NO:11, LCDR1 comprises SEQ ID NO: 4, LCDR2 comprises SEQ ID NO:5, LCDR3 comprises SEQ ID NO:6, HCDR4 comprises SEQ ID NO: 19, HCDR5 comprises SEQ ID NO:20, HCDR6 comprises SEQ ID NO:21, LCDR4 comprises SEQ ID NO: 4, LCDR5 comprises SEQ ID NO:5, LCDR6 comprises SEQ ID NO:6; or (g) HCDR1 comprises SEQ ID NO:1, HCDR2 comprises SEQ ID NO: 7, HCDR3 comprises SEQ ID NO:12, LCDR1 comprises SEQ ID NO:4, LCDR2 comprises SEQ ID NO:5, LCDR3 comprises SEQ ID NO:6, HCDR4 comprises SEQ ID NO: 19, HCDR5 comprises SEQ ID NO:20, HCDR6 comprises SEQ ID NO:21, LCDR4 comprises SEQ ID NO:4, LCDR5 comprises SEQ ID NO:5, LCDR6 comprises SEQ ID NO:6. See Table 2.
16. The bispecific antibody of any one of embodiments 1-2 or 15, wherein: (a) the VH1 comprises SEQ ID NO:22 and the VL1 comprises SEQ ID NO:23 and the VH2 comprises SEQ ID NO: 27 and the VL2 comprises SEQ ID NO:23; (b) the VH1 comprises SEQ ID NO:22 and the VL1 comprises SEQ ID NO: 23 and the VH2 comprises SEQ ID NO:28 and the VL2 comprises SEQ ID NO:23; (c) the VH1 comprises SEQ ID NO:24 and the VL1 comprises SEQ ID NO: 23 and the VH2 comprises SEQ ID NO: 45 and the VL2 comprises SEQ ID NO:23; (d) the VH1 comprises SEQ ID NO:25 and the VL1 comprises SEQ ID NO: 23 and the VH2 comprises SEQ ID NO:28 and the VL2 comprises SEQ ID NO:23; (e) the VH1 comprises SEQ ID NO:22 and the VL1 comprises SEQ ID NO: 23 and the VH2 comprises SEQ ID NO: 45 and the VL2 comprises SEQ ID NO:23; (f) the VHI comprises SEQ ID NO:25 and the VL1 comprises SEQ ID NO: 23 and the VH2 comprises SEQ ID NO:45 and the VL2 comprises SEQ ID NO:23; or (g) the VHI comprises SEQ ID NO:26 and the VL1 comprises SEQ ID NO: 23 and the VH2 comprises SEQ ID NO:28 and the VL2 comprises SEQ ID NO:23. See Table 2.
17. The bispecific antibody of any one of embodiments 1-16, further comprising an Fc region comprising (a) an FGFR3IIIb S249C heavy chain constant region comprising a first C_{H}2 and a first C_{H}3 domains and (b) a CD3 heavy chain constant region comprising a second C_{H}2 and a second C_{H}3 domains, wherein the first C_{H}3 domain has amino acid substitutions L351D and L368E and the second C_{H}3 domain has amino acid substitutions L351K and T366K, according to the Kabat numbering system.
18. The bispecific antibody of any one of embodiments 1-17, further comprising an Fc region comprising (a) an FGFR3IIIb S249C heavy chain constant region comprising a first C_{H}2 and a first C_{H}3 domains and (b) a CD3 heavy chain constant region comprising a second C_{H}2 and a second C_{H}3 domains, wherein the first C_{H}3 domain has amino acid substitutions T366S/L368A/Y407V and the second C_{H}3 domain has amino acid substitution T366W, according to the Kabat numbering system.
19. The bispecific antibody of any one of embodiments 17 or 18, wherein the Fc region comprises Fc silencing mutations.
20. The bispecific antibody of embodiment 19, wherein the Fc silencing mutations are (a) L235G and G236R or (b) L234A, L235A or (c) L234A, L235A and D265A or (d) L234A, L235A and P329G or (e) N297A or (f) N297A and K322A or (g) L234A, L235A and D265S, each according to the Kabat numbering system.
21. The bispecific antibody of any one of embodiments 1-20, wherein the antibody comprises a first heavy chain (HC1) that specifically binds human FGFR3IIIb S249C; a second heavy chain (HC2) that specifically binds human CD3; and a common light chain (cLC) wherein: (a) HC1 comprises SEQ ID NO: 32, HC2 comprises SEQ ID NO:33, cLC comprises SEQ ID NO:37; (b) HC1 comprises SEQ ID NO: 32, HC2 comprises SEQ ID NO:39, cLC comprises SEQ ID NO:37; (c) HC1 comprises SEQ ID NO: 34, HC2 comprises SEQ ID NO:38, cLC comprises SEQ ID NO:37; (d) HC1 comprises SEQ ID NO: 35, HC2 comprises SEQ ID NO:39, cLC comprises SEQ ID NO:37; (e) HC1 comprises SEQ ID NO:32, HC2 comprises SEQ ID NO:38, cLC comprises SEQ ID NO:37; (f) HC1 comprises SEQ ID NO:35, HC2 comprises SEQ ID NO:38, cLC comprises SEQ ID NO:37; or (g) HC1 comprises SEQ ID NO:36, HC2 comprises SEQ ID NO:38, cLC comprises SEQ ID NO:37. See Table 2.
22. The bispecific antibody of any one of embodiments 1-21, wherein the antibody is a human IgG1 or IgG4 isotype.
23. The bispecific antibody of embodiment 22, wherein the antibody is a human IgG1 isotype.
24. A nucleic acid encoding the amino acid sequences of embodiment 21.
25. A host cell transfected with: a first vector comprising the nucleic acids encoding SEQ ID NO: 32, and a second vector comprising the nucleic acids encoding SEQ ID NO:33; a first vector comprising the nucleic acids encoding SEQ ID NO: 32, and a second vector comprising the nucleic acids encoding SEQ ID NO:39; a first vector comprising the nucleic acids encoding SEQ ID NO: 34, and a second vector comprising the nucleic acids encoding SEQ ID NO:38; a first vector comprising the nucleic acids encoding SEQ ID NO: 35, and a second vector comprising the nucleic acids encoding SEQ ID NO:39; a first vector comprising the nucleic acids encoding SEQ ID NO: 32, and a second vector comprising the nucleic acids encoding SEQ ID NO:38; a first vector comprising the nucleic acids encoding SEQ ID NO: 35, and a second vector comprising the nucleic acids encoding SEQ ID NO:38; a first vector comprising the nucleic acids encoding SEQ ID NO: 36, and a second vector comprising the nucleic acids encoding SEQ ID NO:38. See Table 2.
26. The host cell of embodiment 25, further transfected with a third vector comprising the nucleic acids encoding SEQ ID NO:37. See Table 2.
27. The host cell of embodiment 25 or 26, wherein the host cell is a mammalian host cell.
28. A process of producing a bispecific antibody comprising culturing the cell of embodiment 27 in a culture medium under conditions such that the bispecific antibody is expressed, and then recovering the bispecific antibody from the culture medium.
29. A pharmaceutical composition comprising the hFGFR3IIIb S249C×hCD3 bispecific antibody of any one of embodiments 1-23, and a pharmaceutically acceptable excipient, diluent or carrier.
30. An isolated antibody that specifically binds hFGFR3IIIb S249C, wherein the antibody comprises a heavy chain variable region (VH) and a light chain variable region (VL), wherein the VH comprises heavy chain complementarity determining regions (HCDR): HCDR1, HCDR2, and HCDR3, and the VL comprises light chain complementarity determining regions (LCDR): LCDR1, LCDR2, and LCDR3, wherein: (a) HCDR1 comprises SEQ ID NO:1, HCDR2 comprises SEQ ID NO:2, HCDR3 comprises SEQ ID NO:3, LCDR1 comprises SEQ ID NO:4, LCDR2 comprises SEQ ID NO:5, LCDR3 comprises SEQ ID NO:6; (b) HCDR1 comprises SEQ ID NO: 1, HCDR2 comprises SEQ ID NO:7, HCDR3 comprises SEQ ID NO:8, LCDR1 comprises SEQ ID NO:4, LCDR2 comprises SEQ ID NO:5, LCDR3 comprises SEQ ID NO:6; (c) HCDR1 comprises SEQ ID NO:9, HCDR2 comprises SEQ ID NO:10, HCDR3 comprises SEQ ID NO:11, LCDR1 comprises SEQ ID NO:4, LCDR2 comprises SEQ ID NO:5, LCDR3 comprises SEQ ID NO:6; or (d) HCDR1 comprises SEQ ID NO:1, HCDR2 comprises SEQ ID NO: 7, HCDR3 comprises SEQ ID NO: 12, LCDR1 comprises SEQ ID NO:4, LCDR2 comprises SEQ ID NO: 5, LCDR3 comprises SEQ ID NO:6. See Table 1.
31. The antibody of embodiment 30, wherein: (a) the VH comprises SEQ ID NO: 22 and the VL comprises SEQ ID NO: 23; (b) the VH comprises SEQ ID NO: 24 and the VL comprises SEQ ID NO:23; (c) the VH comprises SEQ ID NO: 25 and the VL comprises SEQ ID NO: 23; or (d) the VH comprises SEQ ID NO: 26 and the VL comprises SEQ ID NO: 23. See Table 1.
32. The antibody of embodiment 30 or 31, wherein the antibody comprises a heavy chain (HC) and a light chain (LC), wherein: (a) the HC comprises SEQ ID NO:40 and the LC comprises SEQ ID NO: 37; (b) the HC comprises SEQ ID NO:41 and the LC comprises SEQ ID NO:37; (c) the HC comprises SEQ ID NO:42 and the LC comprises SEQ ID NO:37; or (d) the HC comprises SEQ ID NO:43 and the LC comprises SEQ ID NO:37. See Table 1.
33. The antibody of any one of embodiments 30-32, wherein the antibody is a human IgG1 or lgG4 isotype.
34. The antibody of embodiment 33, wherein the antibody is a human IgG1 isotype.
35. The antibody of any one of embodiments 30-34, wherein the antibody is an antibody fragment or antigen-binding fragment.
36. The antibody of embodiment 35, wherein the antibody fragment or antigen-binding fragment is a Fab, a Fab′, an F(ab′) 2, a single-chain variable fragment (scFv), an Fv, a disulfide-linked Fv (sdFv), an Fd fragment, or a single-chain Fab (scFab).
37. The antibody of any one of embodiments 30-34, wherein the Fc region comprises Fc silencing mutations.
38. The antibody of claim 37, wherein the Fc silencing mutations are (a) L235G and G236R or (b) L234A, L235A or (c) L234A, L235A and D265A or (d) L234A, L235A and P329G or (e) N297A or (f) N297A and K322A or (g) L234A, L235A and D265S, each according to the Kabat numbering system.
39. The antibody of any one of embodiments 30-38, wherein the antibody is a multispecific antibody.
40. The antibody of embodiment 39, wherein the multispecific antibody is a bispecific antibody, or a trispecific antibody, or a tetraspecific antibody, or a diabody, or a tandem scFv, or a tandem VHH, or a tandem scFab.
41. An antibody-drug conjugate (ADC) comprising the antibody of any one of embodiments 30-40 and a drug moiety.
42. The ADC of embodiment 41, wherein the drug moiety is selected from the group consisting of auristatin, N-acetyl-γ calicheamicin, maytansinoid, pyrrolobenzodiazepine, exatecan and SN-38.
43. An immunocytokine comprising the antibody of any one of embodiments 30-40 and a cytokine.
44. The immunocytokine of embodiment 43, wherein the cytokine is selected from the group consisting of IL-2, IL-4, IL-10, IL-12, IL-15, TNF, and IFNα.
45. A chimeric antigen receptor (CAR) comprising the antibody of any one of embodiments 30-40, a transmembrane domain, and an intracellular signaling domain.
46. A pharmaceutical composition comprising the hFGFR3IIIb S249C antibody of any one of embodiments 30-40 or the ADC of any one of embodiments 41-42 or the immunocytokine of any of embodiments 43-44 or the CAR of embodiment 45 and a pharmaceutically acceptable excipient, diluent or carrier.
47. A method of treating an FGFR3IIIb S249C positive cancer in a subject in need thereof, comprising administering to the subject a therapeutically effective amount of (a) the bispecific antibody of any one of embodiments 1-23 and 28, (b) the antibody of any one of embodiments 30-40, (c) the ADC of embodiments 41-42, (d) the immunocytokine of embodiments 43-44, or (e) the CAR of embodiment 45.
48. The method of embodiment 47, wherein the FGFR3IIIb S249C positive cancer is relapsed or refractory.
49. The method of embodiment 47, wherein the FGFR3IIIb S249C positive cancer is metastatic.
50. The method of embodiment 48 or 49, wherein the FGFR3IIIb S249C positive cancer is bladder cancer, non-muscle invasive bladder cancer (NMIBC), muscle invasive bladder cancer (MIBC), urothelial carcinoma, papillary urothelial carcinomas, infiltrating renal pelvis and ureter urothelial carcinoma, urethral urothelial carcinoma, urinary tract cancer, renal cell carcinoma, colorectal carcinoma, pancreatic exocrine carcinoma, lung cancer, including non-small cell lung cancer (NSCLC) and squamous cell lung carcinoma, skin cancer, cervical cancer, ovarian cancer, endometrial adenocarcinoma, HPV-positive vulvar squamous cell carcinoma (VSCC), prostate cancer, gastric adenocarcinoma, esophageal cancer, upper aero-digestive tract (UAT) cancer, glioma, and head and neck squamous cell carcinoma.
51. The method of any one of embodiments 47-50, wherein the bispecific antibody of any one of embodiments 1-23 and 28 induces an in vivo human IFNγ concentration of about 0.10 μg/ml or less, or about 0.09 μg/ml or less, or about 0.08 μg/ml or less, about 0.07 μg/ml or less, about 0.06 μg/ml or less, about 0.05 μg/ml or less, about 0.04 μg/ml or less, about 0.03 μg/ml or less, about 0.02 μg/ml or less, about 0.01 μg/ml or less, as measured, for example without limitation, by an immunoassay analyte detection system. In another embodiment, the bispecific antibody of any one of embodiments 1-23 and 28 does not induce any measurable human IFNγ concentration as measured, for example without limitation, by an immunoassay analyte detection system. See Example 17.
52. The method of any one of embodiments 47-51, wherein the bispecific antibody of any one of embodiments 1-23 and 28 induces an in vivo human IL-2 concentration of about 0.10 μg/ml or less, or about 0.09 μg/ml or less, or about 0.08 μg/ml or less, about 0.07 μg/ml or less, about 0.6 μg/ml or less, about 0.05 μg/ml or less, about 0.04 μg/ml or less, about 0.03 μg/ml or less, about 0.02 μg/ml or less, about 0.01 μg/ml or less as measured, for example without limitation, by an immunoassay analyte detection system. See Example 17. In a further embodiment, the bispecific antibody of any one of embodiments 1-23 and 28 does not induce any measurable human IL-10 or tumor necrosis factor (TNF) as measured, for example without limitation, by an immunoassay analyte detection system.
53. The bispecific antibody of any one of embodiments 1-23 and 28 for use in the treatment of an FGFR3IIIb S249C positive cancer.
54. The antibody of any one of embodiments 30-40 for use in the treatment of an FGFR3IIIb S249C positive cancer.
55. The ADC of embodiments 41-42 for use in the treatment of an FGFR3IIIb S249C positive cancer.
56. The immunocytokine of embodiments 43-44 for use in the treatment of an FGFR3IIIb S249C positive cancer.
57. The CAR of embodiment 45 for use in the treatment of an FGFR3IIIb S249C positive cancer.
58. The use of any one of embodiments 53-57, wherein the FGFR3IIIb S249C positive is relapsed or refractory.
59. The use of any one of embodiments 53-57, wherein the FGFR3IIIb S249C positive cancer is metastatic.
60. A pharmaceutical composition comprising the bispecific antibody of any one of embodiments 1-23 and 28 or the antibody of any one of embodiments 30-40 or the ADC of embodiments 41-42 or the immunocytokine of embodiments 43-44 or the CAR of embodiment 45 for use in treating FGFR3IIIb S249C positive cancer.
61. The pharmaceutical composition of embodiment 60, wherein the FGFR3IIIb S249C positive cancer is relapsed or refractory.
62. The pharmaceutical composition of embodiment 60, wherein the FGFR3IIIb S249C positive cancer is metastatic.
63. The method of any one of embodiments 47-52, wherein treatment using the pharmaceutical composition of embodiment 29 demonstrates a tumor regression of at least 70%, at least 75%, at least 80%, at least 85%, at least 90%, at least 95% from baseline when measured at least 14 days, at least 21 days, at least 28 days from initial treatment.
64. Use of the bispecific antibody of any one of embodiments 1-23 and 28 or the antibody of any one of embodiments 30-40 or the ADC of embodiments 41-42 or the immunocytokine of embodiments 43-44 or the CAR of embodiment 45, in the manufacture of a medicament for the treatment of an FGFR3IIIb S249C positive cancer.
65. The use of embodiment 64, wherein the FGFR3IIIb S249C positive cancer is relapsed or refractory.
66. The use of embodiment 65, wherein the FGFR3IIIb S249C positive cancer is metastatic.

### Definitions

The term “antibody,” as used herein, refers to an isolated immunoglobulin molecule that specifically binds an antigen, such as, for example, a tumor antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4), unless otherwise specified.

An exemplary bispecific antibody or T-cell engager (TCE) of the present disclosure is an immunoglobulin G (IgG) type antibody comprised of four polypeptide chains: two heavy chains (HC) and two light chains that are common (cLC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain (HC) is comprised of a heavy chain variable region (VH), a heavy chain constant region (C_{H}1), a hinge region, a heavy chain constant region 2 (CH₂) and a heavy chain constant region 3 (C_{H}3). Each common light chain is comprised of a light chain variable region (VL) and a light chain constant region (LC). The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to schemes well known to those of skill in the art, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212). Unless otherwise specified, the present disclosed CDR sequences herein use the North numbering convention.

Also contemplated are antibody fragments or antigen-binding fragments that, as used herein, comprise at least a portion of an antibody retaining the ability to specifically interact with an antigen or an epitope of the antigen, such as Fab, Fab′, F(ab′)2, Fv fragments, scFv antibody fragments, scFab, disulfide-linked Fvs (sdFv), a Fd fragment.

The term “antigen binding domain”, as used herein, refers to a portion of an antibody, antibody fragment, bispecific antibody, multispecific binding protein, antibody-drug conjugate, immunocytokine, chimeric antigen receptor (CAR) that specifically binds an antigen or an epitope of the antigen.

The term “bispecific”, as used herein, refers to a molecule that comprises two distinct antigen-binding domains. A bispecific binding molecule can bind two different antigens or two different epitopes of the same antigen. Exemplary embodiments of bispecific molecules include the TCEs disclosed herein.

The term “multispecific”, as used herein, refers to a molecule that comprises two or more distinct antigen-binding domains. A multispecific binding molecule can bind two or more different antigens, or two or more different epitopes of the same antigen. Exemplary embodiments of multispecific binding molecules include bispecific, trispecific or tetraspecific binding molecules known in the field, as well as single-chain multispecific binding molecules such as diabodies, tandem scFvs, tandem VHHs, or tandem scFabs.

The term “antibody drug conjugate” (ADC) as used herein refers to a class of compounds comprising (a) an antibody or antibody fragment, (2) a payload, such as a cytotoxic agent, an immunological modulator or therapeutic peptide, and (3) a linker that conjugates the antibody to the payload.

The term “chimeric antigen receptor” (CAR) refers to a recombinant polypeptide construct comprising at least an extracellular antigen binding domain, a transmembrane domain and an intracellular signaling domain.

The term “effective amount” of a bispecific antibody or T-cell engager (TCE) of the invention, for example, a human FGFR3IIIb S249C×human CD3 bispecific antibody of the invention or a composition (e.g., pharmaceutical composition) thereof, is at least the minimum amount required to achieve a desired result, such as a measurable improvement of a particular cancer, such as, for example the FGFR3IIIb positive cancers or tumors described herein. For example, in the case of a cancer or tumor, an effective amount of the bispecific antibody or TCE of the invention may reduce the number of cancer cells present; reduce the tumor size; inhibit, slow or stop cancer cell infiltration into peripheral organs or tumor metastasis or tumor growth. An effective amount is also one in which any beneficial effects of treatment outweigh or mitigate any toxic or detrimental effects of the treatment, such as, for example without any limitation, any cytokine release syndrome.

The term “Fc silencing mutation(s)” as used herein involves mutations in the amino acid sequence of the Fc regions of an antibody where human Fc gamma receptors of effector cells bind to an antibody. These mutations reduce or eliminate effector function while retaining binding to the neonatal Fc receptor, important for normal antibody pharmacokinetics.

The term “FGFR3 isoform IIIb S249C positive cancer” or “FGFR3IIIb S249C positive cancer” or “FGFR3IIIb S249C+ cancer” and the like, as used herein means any cancer or tumor associated with at least the specific amino acid mutation of serine to cysteine at position 249 of the FGFR3 isoform IIIb protein, according to the Kabat numbering scheme. Such cancers include without limitation, bladder cancer, which includes but is not limited to non-muscle invasive bladder cancer (NMIBC) and muscle invasive bladder cancer (MIBC). Other FGFR3IIIb S249C positive cancers may include, but are not limited to, urothelial carcinoma, papillary urothelial carcinomas, infiltrating renal pelvis and ureter urothelial carcinoma, urethral urothelial carcinoma, urinary tract cancer, renal cell carcinoma, colorectal carcinoma, pancreatic exocrine carcinoma, lung cancer, including non-small cell lung cancer (NSCLC) and squamous cell lung carcinoma, skin cancer, cervical cancer, ovarian cancer, endometrial adenocarcinoma, HPV-positive vulvar squamous cell carcinoma (VSCC), prostate cancer, gastric adenocarcinoma, esophageal cancer, upper aero-digestive tract (UAT) cancer, glioma, and head and neck squamous cell carcinoma.

The term “immunocytokine” as used herein is a molecule that comprises an antigen-binding domain connected to a cytokine by chemical conjugation. In some embodiments, the antigen-binding domain is connected to the cytokine by fusion to the cytokine. The immunocytokine can further comprise an Fc domain connected to the antigen-binding domain.

The term “metastatic” or “metastasize” as used herein means a human cancer, including the FGFR3IIIb S249C positive cancers defined herein, that spreads from the site of its origin to another part of the human body.

The terms “nucleic acid” or “polynucleotide”, as used interchangeably herein, refer to polymers of nucleotides, including single-stranded and/or double-stranded nucleotide-containing molecules, such as DNA, cDNA and RNA molecules, incorporating native, modified, and/or analogs of, nucleotides. Polynucleotides of the present disclosure may also include substrates incorporated therein, for example, by DNA or RNA polymerase or a synthetic reaction.

Polynucleotides of the present disclosure may be expressed in a host cell, for example after the polynucleotides have been operably linked to an expression control sequence. Expression control sequences capable of expression of polynucleotides to which they are operably linked are well known in the art. For example, an expression vector may include a sequence that encodes one or more signal peptides that facilitate secretion of the polypeptide(s) from a host cell. The signal peptide may be an immunoglobulin signal peptide or a heterologous signal peptide, for example. Expression vectors containing a polynucleotide of interest (e.g., a polynucleotide encoding a polypeptide of an antibody) may be transferred into a host cell by well-known methods. Additionally, expression vectors may contain one or more selection markers, e.g., tetracycline, neomycin, and dihydrofolate reductase, to aide in detection of host cells transformed with the desired polynucleotide sequences.

A host cell includes cells stably or transiently transfected, transformed, transduced or infected with one or more expression vectors expressing all or a portion of an antibody of the present disclosure. According to some embodiments, a host cell may be stably or transiently transfected, transformed, transduced or infected with expression vector(s) expressing HC polypeptides and an expression vector expressing LC polypeptides of an antibody of the present disclosure. In some embodiments, a host cell may be stably or transiently transfected, transformed, transduced or infected with an expression vector expressing HC and LC polypeptides of an antibody of the present disclosure. The antibody of the present invention may be produced in mammalian cells such as CHO, NSO, HEK293 or COS cells according to techniques well known in the art.

Medium, into which an antibody of the present invention has been secreted, may be purified by conventional techniques, such as mixed-mode methods of ion-exchange and hydrophobic interaction chromatography. For example, the medium may be applied to and eluted from a Protein A or G column using conventional methods; mixed-mode methods of ion-exchange and hydrophobic interaction chromatography may also be used. Soluble aggregate and multimers may be effectively removed by common techniques, including size exclusion, hydrophobic interaction, ion exchange, or hydroxyapatite chromatography. The product may be immediately frozen, for example at −70° C., refrigerated, or may be lyophilized. Various methods of protein purification may be employed, and such methods are known in the art and described, for example, in Deutscher, Methods in Enzymology 182:83-89 (1990) and Scopes, Protein Purification: Principles and Practice, 3rd Edition, Springer, NY (1994).

The term “relapse” or “relapsed” as used herein is when a disease, such as a human cancer, including the FGFR3IIIb S249C positive cancers defined herein, returns following a successful treatment. A synonymous term to relapse is “recurrence”.

The term “refractory” as used herein is when a disease, such as a human cancer, including the FGFR3IIIb S249C positive cancers defined herein, fails to respond to a medical treatment from the start of treatment. A synonymous term to refractory is “resistance”. A refractory disease, such as a refractory cancer is different from a relapsed disease, such as a relapsed cancer, defined above.

The terms “specifically bind” and “specifically binds” as used herein are intended to mean, unless indicated otherwise, the ability of a bispecific TCE of the invention to form a chemical bond or attractive interaction with another protein or molecule, which results in proximity of the antibody and other protein or molecule as determined by common methods known in the art. For example, specific binding of the FGFR3IIIb S249C TCEs of the invention to FGFR3IIIb harboring the S249C mutation can be measured by, for example without limitation, an ELISA assay or by a cell binding assay known in the art where said TCEs will demonstrate binding to the FGFR3IIIb S249C protein but not any measurable binding to WT FGFR3 or to FGFR3 isoform IIIc in either WT form or harboring the S249C mutation. See for example, without limitation, Examples 3-8.

The term “T-cell engager(s)” (TCEs) as used herein refers to a particular type of bispecific antibody where one of the antibody arms specifically binds to a tumor-associated antigen (TAA) and the other antibody arm specifically binds to CD3 such that the TCE engages a cytotoxic T cell through the CD3 arm and redirects the T cell towards a tumor cell expressing the TAA in order for the TCE to form a cytolytic synapse into which the engaged T cell releases pore-forming perforin and cytotoxic granzyme-B, leading to killing of the targeted tumor cell. In one embodiment, TCEs disclosed herein are the human FGFR3IIIb S249C×human CD3 bispecific antibodies of the invention. As used herein the terms “hFGFR3IIIb S249C TCE” and “hFGFR3IIIb S249C×hCD3 bispecific or TCE” and “hFGFR3IIIb S249C×hCD3 bispecific antibody or TCE” and “FGFR3IIIb S249C TCE” are all synonymous unless otherwise indicated.

The term “treating” or “treat” in the context of disease as used herein refer to reducing the severity and/or frequency of one or more symptoms, eliminating one or more symptoms and/or the underlying cause of said symptoms, reducing the frequency or likelihood of one or more symptoms and/or their underlying cause, delaying, preventing and/or slowing the progression of diseases and/or disorders and improving or remediating damage caused, directly or indirectly, by the diseases and/or disorders. For clarity, treating a disease does not include and is separate from preventing a disease.

The sequences of the hFGFR3IIIb S249C antibodies and hFGFR3IIIb S249C×hCD3 TCEs of the invention are numbered according to the sequence identifier numbers listed in Tables 1 and 2, respectively. The sequences in both Tables 1 and 2 are amino acid sequences and the CDR sequences use the North numbering convention, unless otherwise indicated (see North, B. et al., J Mol Biol. 2011 Feb. 18; 406 (2): 228-256).

TABLE 1
hFGFR3IIIb S249C Antibodies of the Invention
HUMAN FGFR3 S249C ANTIBODY CLONE A

| | | |
|---|---|---|
| HCDR1 | TFSGFSLSTSGEGVG | SEQ ID |
| | | NO: 1 |
| HCDR2 | LIYWTDDKR | SEQ ID |
| | | NO: 2 |
| HCDR3 | AHRRGGYSDHDSYYYYYLDV | SEQ ID |
| | | NO: 3 |
| LCDR1 | RASQSISSYLN | SEQ ID |
| | | NO: 4 |
| LCDR2 | YAASSLQS | SEQ ID |
| | | NO: 5 |
| LCDR3 | QQSYSTPPT | SEQ ID |
| | | NO: 6 |

| | |
|---|---|
| Variable Heavy (VH): | SEQ ID |
| QITLKESGPTLVKPTQTLTLTCTFSGFSLSTSGEGVGWIRQPPGKALEWLALIYWTD | NO: 22 |
| DKRYSPSLKSRLTITKDTSKNHVVLTMTNMDPVDTATYYCAHRRGGYSDHDSYY | |
| YYYLDVWGKGTTVTVSS | |
| Variable Light (VL): | SEQ ID |
| DIQMTQSPSSLSASVGDRVTITCRASQSISSYLNWYQQKPGKAPKLLIYAASSLQS | NO: 23 |
| GVPSRFSGSGSGTDFTLTISSLQPEDFATYYCQQSYSTPPTFGQGTKVEIK | |
| Constant Heavy (CH): | SEQ ID |
| ASTKGPSVFPLAPSSKSTSGGTAALGCLVKDYFPEPVTVSWNSGALTSGVHTFPA | NO: 29 |
| VLQSSGLYSLSSVVTVPSSSLGTQTYICNVNHKPSNTKVDKKVEPKSCDKTHTCPP | |
| CPAPELLGGPSVFLFPPKPKDTLMISRTPEVTCVVVDVSHEDPEVKFNWYVDGVE | |
| VHNAKTKPREEQYNSTYRVVSVLTVLHQDWLNGKEYKCKVSNKALPAPIEKTIS | |
| KAKGQPREPQVYTLPPSRDELTKNQVSLTCLVKGFYPSDIAVEWESNGQPENNY | |
| KTTPPVLDSDGSFFLYSKLTVDKSRWQQGNVFSCSVMHEALHNHYTQKSLSLSP | |
| GK | |
| Constant Light (CL): | SEQ ID |
| RTVAAPSVFIFPPSDEQLKSGTASVVCLLNNFYPREAKVQWKVDNALQSGNSQES | NO: 31 |
| VTEQDSKDSTYSLSSTLTLSKADYEKHKVYACEVTHQGLSSPVTKSFNRGEC | |
| Heavy Chain (HC): | SEQ ID |
| QITLKESGPTLVKPTQTLTLTCTFSGFSLSTSGEGVGWIRQPPGKALEWLALIYWT | NO: 40 |
| DDKRYSPSLKSRLTITKDTSKNHVVLTMTNMDPVDTATYYCAHRRGGYSDHDS | |
| YYYYYLDVWGKGTTVTVSSASTKGPSVFPLAPSSKSTSGGTAALGCLVKDYFPEP | |
| VTVSWNSGALTSGVHTFPAVLQSSGLYSLSSVVTVPSSSLGTQTYICNVNHKPSN | |
| TKVDKKVEPKSCDKTHTCPPCPAPELLGGPSVFLFPPKPKDTLMISRTPEVTCVVV | |
| DVSHEDPEVKFNWYVDGVEVHNAKTKPREEQYNSTYRVVSVLTVLHQDWLNG | |
| KEYKCKVSNKALPAPIEKTISKAKGQPREPQVYTLPPSRDELTKNQVSLTCLVKGF | |
| YPSDIAVEWESNGQPENNYKTTPPVLDSDGSFFLYSKLTVDKSRWQQGNVFSCSV | |
| MHEALHNHYTQKSLSLSPGK | |
| Common Light Chain (cLC): | SEQ ID |
| DIQMTQSPSSLSASVGDRVTITCRASQSISSYLNWYQQKPGKAPKLLIYAASSLQS | NO: 37 |
| GVPSRFSGSGSGTDFTLTISSLQPEDFATYYCQQSYSTPPTFGQGTKVEIKRTVAAPS | |
| VFIFPPSDEQLKSGTASVVCLLNNFYPREAKVQWKVDNALQSGNSQESVTEQDSK | |
| DSTYSLSSTLTLSKADYEKHKVYACEVTHQGLSSPVTKSFNRGEC | |

HUMAN FGFR3 S249C ANTIBODY CLONE B

| | | |
|---|---|---|
| HCDR1 | TFSGFSLSTSGEGVG | SEQ ID |
| | | NO: 1 |
| HCDR2 | LIYWSDDKR | SEQ ID |
| | | NO: 7 |
| HCDR3 | AHRRGGYSDHDSYYYYYMDV | SEQ ID |
| | | NO: 8 |
| LCDR1 | RASQSISSYLN | SEQ ID |
| | | NO: 4 |
| LCDR2 | YAASSLQS | SEQ ID |
| | | NO: 5 |
| LCDR3 | QQSYSTPPT | SEQ ID |
| | | NO: 6 |

| | |
|---|---|
| Variable Heavy (VH): | SEQ ID |
| QITLKESGPTLVKPTQTLTLTCTFSGFSLSTSGEGVGWIRQPPGKALEWLALIYWSD | NO: 24 |
| DKRYSPSLKNRLTITKDTSKNQVVLTMTNMDPVDTATYYCAHRRGGYSDHDSYY | |
| YYYMDVWGKGTMVTVSS | |
| Variable Light (VL): | SEQ ID |
| DIQMTQSPSSLSASVGDRVTITCRASQSISSYLNWYQQKPGKAPKLLIYAASSLQS | NO: 23 |
| GVPSRFSGSGSGTDFTLTISSLQPEDFATYYCQQSYSTPPTFGQGTKVEIK | |
| Constant Heavy (CH): | SEQ ID |
| ASTKGPSVFPLAPSSKSTSGGTAALGCLVKDYFPEPVTVSWNSGALTSGVHTFPAV | NO: 29 |
| LQSSGLYSLSSVVTVPSSSLGTQTYICNVNHKPSNTKVDKKVEPKSCDKTHTCPPC | |
| PAPELLGGPSVFLFPPKPKDTLMISRTPEVTCVVVDVSHEDPEVKFNWYVDGVEV | |
| HNAKTKPREEQYNSTYRVVSVLTVLHQDWLNGKEYKCKVSNKALPAPIEKTISK | |
| AKGQPREPQVYTLPPSRDELTKNQVSLTCLVKGFYPSDIAVEWESNGQPENNYKT | |
| TPPVLDSDGSFFLYSKLTVDKSRWQQGNVFSCSVMHEALHNHYTQKSLSLSPGK | |
| Constant Light (CL): | SEQ ID |
| RTVAAPSVFIFPPSDEQLKSGTASVVCLLNNFYPREAKVQWKVDNALQSGNSQES | NO: 31 |
| VTEQDSKDSTYSLSSTLTLSKADYEKHKVYACEVTHQGLSSPVTKSFNRGEC | |
| Heavy Chain (HC): | SEQ ID |
| QITLKESGPTLVKPTQTLTLTCTFSGFSLSTSGEGVGWIRQPPGKALEWLALIYWSD | NO: 41 |
| DKRYSPSLKNRLTITKDTSKNQVVLTMTNMDPVDTATYYCAHRRGGYSDHDSYY | |
| YYYMDVWGKGTMVTVSSASTKGPSVFPLAPSSKSTSGGTAALGCLVKDYFPEPV | |
| TVSWNSGALTSGVHTFPAVLQSSGLYSLSSVVTVPSSSLGTQTYICNVNHKPSNTK | |
| VDKKVEPKSCDKTHTCPPCPAPELLGGPSVFLFPPKPKDTLMISRTPEVTCVVVDV | |
| SHEDPEVKFNWYVDGVEVHNAKTKPREEQYNSTYRVVSVLTVLHQDWLNGKEY | |
| KCKVSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCSVMHE | |
| ALHNHYTQKSLSLSPGK | |
| Common Light Chain (cLC): | SEQ ID |
| DIQMTQSPSSLSASVGDRVTITCRASQSISSYLNWYQQKPGKAPKLLIYAASSLQS | NO: 37 |
| GVPSRFSGSGSGTDFTLTISSLQPEDFATYYCQQSYSTPPTFGQGTKVEIKRTVAAPS | |
| VFIFPPSDEQLKSGTASVVCLLNNFYPREAKVQWKVDNALQSGNSQESVTEQDSK | |
| DSTYSLSSTLTLSKADYEKHKVYACEVTHQGLSSPVTKSFNRGEC | |

HUMAN FGFR3 S249C ANTIBODY CLONE C

| | | |
|---|---|---|
| HCDR1 | TFSGFSLSTSGEAVG | SEQ ID |
| | | NO: 9 |
| HCDR2 | LIYWSDDSR | SEQ ID |
| | | NO: 10 |
| HCDR3 | AHRRGSYSGDYYYYYMDV | SEQ ID |
| | | NO: 11 |
| LCDR1 | RASQSISSYLN | SEQ ID |
| | | NO: 4 |
| LCDR2 | YAASSLQS | SEQ ID |
| | | NO: 5 |
| LCDR3 | QQSYSTPPT | SEQ ID |
| | | NO: 6 |

| | |
|---|---|
| Variable Heavy (VH): | SEQ ID |
| QITLKESGPTLVKPTQTLTLTCTFSGFSLSTSGEAVGWIRQPPGKALEWLALIYWSD | NO: 25 |
| DSRYSPSLKSRLTITRVTAKNQVVLKMTNMDPVDTATYYCAHRRGSYSGDYYYY | |
| YMDVWGKGTMVTVSS | |
| Variable Light (VL): | SEQ ID |
| DIQMTQSPSSLSASVGDRVTITCRASQSISSYLNWYQQKPGKAPKLLIYAASSLQS | NO: 23 |
| GVPSRFSGSGSGTDFTLTISSLQPEDFATYYCQQSYSTPPTFGQGTKVEIK | |
| Constant Heavy (CH): | SEQ ID |
| ASTKGPSVFPLAPSSKSTSGGTAALGCLVKDYFPEPVTVSWNSGALTSGVHTFPAV | NO: 29 |
| LQSSGLYSLSSVVTVPSSSLGTQTYICNVNHKPSNTKVDKKVEPKSCDKTHTCPPC | |
| PAPELLGGPSVFLFPPKPKDTLMISRTPEVTCVVVDVSHEDPEVKFNWYVDGVEV | |
| HNAKTKPREEQYNSTYRVVSVLTVLHQDWLNGKEYKCKVSNKALPAPIEKTISK | |
| AKGQPREPQVYTLPPSRDELTKNQVSLTCLVKGFYPSDIAVEWESNGQPENNYKT | |
| TPPVLDSDGSFFLYSKLTVDKSRWQQGNVFSCSVMHEALHNHYTQKSLSLSPGK | |
| Constant Light (CL): | SEQ ID |
| RTVAAPSVFIFPPSDEQLKSGTASVVCLLNNFYPREAKVQWKVDNALQSGNSQES | NO: 31 |
| VTEQDSKDSTYSLSSTLTLSKADYEKHKVYACEVTHQGLSSPVTKSFNRGEC | |
| Heavy Chain (HC): | SEQ ID |
| QITLKESGPTLVKPTQTLTLTCTFSGFSLSTSGEAVGWIRQPPGKALEWLALIYWSD | NO: 42 |
| DSRYSPSLKSRLTITRVTAKNQVVLKMTNMDPVDTATYYCAHRRGSYSGDYYYY | |
| YMDVWGKGTMVTVSSASTKGPSVFPLAPSSKSTSGGTAALGCLVKDYFPEPVTV | |
| SWNSGALTSGVHTFPAVLQSSGLYSLSSVVTVPSSSLGTQTYICNVNHKPSNTKVD | |
| KKVEPKSCDKTHTCPPCPAPELLGGPSVFLFPPKPKDTLMISRTPEVTCVVVDVSH | |
| EDPEVKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVMHEAL | |
| HNHYTQKSLSLSPGK | |
| Common Light Chain (cLC): | SEQ ID |
| DIQMTQSPSSLSASVGDRVTITCRASQSISSYLNWYQQKPGKAPKLLIYAASSLQS | NO: 37 |
| GVPSRFSGSGSGTDFTLTISSLQPEDFATYYCQQSYSTPPTFGQGTKVEIKRTVAAPS | |
| VFIFPPSDEQLKSGTASVVCLLNNFYPREAKVQWKVDNALQSGNSQESVTEQDSK | |
| DSTYSLSSTLTLSKADYEKHKVYACEVTHQGLSSPVTKSFNRGEC | |

HUMAN FGFR3 S249C ANTIBODY CLONE D

| | | |
|---|---|---|
| HCDR1 | TFSGFSLSTSGEGVG | SEQ ID |
| | | NO: 1 |
| HCDR2 | LIYWSDDKR | SEQ ID |
| | | NO: 7 |
| HCDR3 | AHRRGGYSGYDSYYYYYMDV | SEQ ID |
| | | NO: 12 |
| LCDR1 | RASQSISSYLN | SEQ ID |
| | | NO: 4 |
| LCDR2 | YAASSLQS | SEQ ID |
| | | NO: 5 |
| LCDR3 | QQSYSTPPT | SEQ ID |
| | | NO: 6 |

| | |
|---|---|
| Variable Heavy (VH): | SEQ ID |
| RITLKESGPTLVKPTQTLTLTCTFSGFSLSTSGEGVGWIRQPPGKALEWLALIYWSD | NO: 26 |
| DKRYSPSLKNRLTITKDTSKNQVVLTMTNMDPVDTATYYCAHRRGGYSGYDSYY | |
| YYYMDVWGKGTMVTVSS | |
| Variable Light (VL): | SEQ ID |
| DIQMTQSPSSLSASVGDRVTITCRASQSISSYLNWYQQKPGKAPKLLIYAASSLQS | NO: 23 |
| GVPSRFSGSGSGTDFTLTISSLQPEDFATYYCQQSYSTPPTFGQGTKVEIK | |
| Constant Heavy (CH): | SEQ ID |
| ASTKGPSVFPLAPSSKSTSGGTAALGCLVKDYFPEPVTVSWNSGALTSGVHTFPAV | NO: 29 |
| LQSSGLYSLSSVVTVPSSSLGTQTYICNVNHKPSNTKVDKKVEPKSCDKTHTCPPC | |
| PAPELLGGPSVFLFPPKPKDTLMISRTPEVTCVVVDVSHEDPEVKFNWYVDGVEV | |
| HNAKTKPREEQYNSTYRVVSVLTVLHQDWLNGKEYKCKVSNKALPAPIEKTISK | |
| AKGQPREPQVYTLPPSRDELTKNQVSLTCLVKGFYPSDIAVEWESNGQPENNYKT | |
| TPPVLDSDGSFFLYSKLTVDKSRWQQGNVFSCSVMHEALHNHYTQKSLSLSPGK | |
| Constant Light (CL): | SEQ ID |
| RTVAAPSVFIFPPSDEQLKSGTASVVCLLNNFYPREAKVQWKVDNALQSGNSQES | NO: 31 |
| VTEQDSKDSTYSLSSTLTLSKADYEKHKVYACEVTHQGLSSPVTKSFNRGEC | |
| Heavy Chain (HC): | SEQ ID |
| RITLKESGPTLVKPTQTLTLTCTFSGFSLSTSGEGVGWIRQPPGKALEWLALIYWSD | NO: 43 |
| DKRYSPSLKNRLTITKDTSKNQVVLTMTNMDPVDTATYYCAHRRGGYSGYDSYY | |
| YYYMDVWGKGTMVTVSSASTKGPSVFPLAPSSKSTSGGTAALGCLVKDYFPEPV | |
| TVSWNSGALTSGVHTFPAVLQSSGLYSLSSVVTVPSSSLGTQTYICNVNHKPSNTK | |
| VDKKVEPKSCDKTHTCPPCPAPELLGGPSVFLFPPKPKDTLMISRTPEVTCVVVDV | |
| SHEDPEVKFNWYVDGVEVHNAKTKPREEQYNSTYRVVSVLTVLHQDWLNGKEY | |
| KCKVSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCSVMHE | |
| ALHNHYTQKSLSLSPGK | |
| Common Light Chain (cLC): | SEQ ID |
| DIQMTQSPSSLSASVGDRVTITCRASQSISSYLNWYQQKPGKAPKLLIYAASSLQS | NO: 37 |
| GVPSRFSGSGSGTDFTLTISSLQPEDFATYYCQQSYSTPPTFGQGTKVEIKRTVAAPS | |
| VFIFPPSDEQLKSGTASVVCLLNNFYPREAKVQWKVDNALQSGNSQESVTEQDSK | |
| DSTYSLSSTLTLSKADYEKHKVYACEVTHQGLSSPVTKSFNRGEC | |

TABLE 2
hFGFR3IIIb S249C x hCD3 Bispecific Antibodies of the Invention
TCE 1

| HUMAN hFGFR3IIIb S249C CLONE A | | HUMAN CD3 CLONE A | |
|---|---|---|---|
| HCDR1: | SEQ ID | HCDR4: | SEQ ID |
| TFSGFSLSTSGEGVG | NO: 1 | KASGDAFKSKTFTIS | NO: 13 |
| HCDR2: | SEQ ID | HCDR5: | SEQ ID |
| LIYWTDDKR | NO: 2 | GIIPVFGTIT | NO: 14 |
| HCDR3: | SEQ ID | HCDR6: | SEQ ID |
| AHRRGGYSDHDSYYYYYLDV | NO: 3 | TRRGKWNPFDP | NO: 15 |
| LCDR1: | SEQ ID | LCDR4: | SEQ ID |
| RASQSISSYLN | NO: 4 | RASQSISSYLN | NO: 4 |
| LCDR2: | SEQ ID | LCDR5: | SEQ ID |
| YAASSLQS | NO: 5 | YAASSLQS | NO: 5 |
| LCDR3: | SEQ ID | LCDR6: | SEQ ID |
| QQSYSTPPT | NO: 6 | QQSYSTPPT | NO: 6 |
| VH1: | SEQ ID | VH2: | SEQ ID |
| QITLKESGPTLVKPTQTLTLTCTFSGFSL | NO: 22 | EVQLVQSGAEVKKPGSSVKVSCKAS | NO: 27 |
| STSGEGVGWIRQPPGKALEWLALIYW | | GDAFKSKTFTISWVRQAPGQGLEW | |
| TDDKRYSPSLKSRLTITKDTSKNHVVLT | | LGGIIPVFGTITYAQKFQGRVTITADK | |
| MTNMDPVDTATYYCAHRRGGYSDHD | | STNTAFMELSSLRSEDTAIYYCTRRG | |
| SYYYYYLDVWGKGTTVTVSS | | KWNPFDPWGPGTLVTVSS | |
| VL1 and 2: | | | SEQ ID |
| DIQMTQSPSSLSASVGDRVTITCRASQSISSYLNWYQQKPGKAPKLLIYAASSLQSGVPSRFS | | | NO: 23 |
| GSGSGTDFTLTISSLQPEDFATYYCQQSYSTPPTFGQGTKVEIK | | | |
| Constant Heavy (CH): | SEQ ID | CH2: | SEQ ID |
| ASTKGPSVFPLAPSSKSTSGGTAALGC | NO: 44 | ASTKGPSVFPLAPSSKSTSGGTAALG | NO: 30 |
| LVKDYFPEPVTVSWNSGALTSGVHTFP | | CLVKDYFPEPVTVSWNSGALTSGVH | |
| AVLQSSGLYSLSSVVTVPSSSLGTQTYI | | TFPAVLQSSGLYSLSSVVTVPSSSLGT | |
| CNVNHKPSNTKVDKKVEPKSCDKTHT | | QTYICNVNHKPSNTKVDKKVEPKSC | |
| CPPCPAPEAAGGPSVFLFPPKPKDTLMI | | DKTHTCPPCPAPEAAGGPSVFLFPPK | |
| SRTPEVTCVVVSVSHEDPEVKFNWYV | | PKDTLMISRTPEVTCVVVSVSHEDPE | |
| DGVEVHNAKTKPREEQYNSTYRVVSV | | VKFNWYVDGVEVHNAKTKPREEQ | |
| LTVLHQDWLNGKEYKCKVSNKALPAP | | YNSTYRVVSVLTVLHQDWLNGKEY | |
| IEKTISKAKGQPREPQVYTLPPSRDELT | | KCKVSNKALPAPIEKTISKAKGQPRE | |
| KNQVSLSCAVKGFYPSDIAVEWESNG | | PQVYTLPPSRDELTKNQVSLWCLVK | |
| QPENNYKTTPPVLDSDGSFFLVSKLTV | | GFYPSDIAVEWESNGQPENNYKTTP | |
| DKSRWQQGNVFSCSVMHEALHNHYT | | PVLDSDGSFFLYSKLTVDKSRWQQG | |
| QKSLSLSPGK | | NVFSCSVMHEALHNHYTQKSLSLSP | |
| | | GK | |
| Constant Light (CL): | | | SEQ ID |
| RTVAAPSVFIFPPSDEQLKSGTASVVCLLNNFYPREAKVQWKVDNALQSGNSQESVTEQD | | | NO: 31 |
| SKDSTYSLSSTLTLSKADYEKHKVYACEVTHQGLSSPVTKSFNRGEC | | | |
| HC1: | SEQ ID | HC2: | SEQ ID |
| QITLKESGPTLVKPTQTLTLTCTFSGFSL | NO: 32 | EVQLVQSGAEVKKPGSSVKVSCKAS | NO: 33 |
| STSGEGVGWIRQPPGKALEWLALIYW | | GDAFKSKTFTISWVRQAPGQGLEW | |
| TDDKRYSPSLKSRLTITKDTSKNHVVLT | | LGGIIPVFGTITYAQKFQGRVTITADK | |
| MTNMDPVDTATYYCAHRRGGYSDHD | | STNTAFMELSSLRSEDTAIYYCTRRG | |
| SYYYYYLDVWGKGTTVTVSSASTKGP | | KWNPFDPWGPGTLVTVSSASTKGPS | |
| SVFPLAPSSKSTSGGTAALGCLVKDYFP | | VFPLAPSSKSTSGGTAALGCLVKDY | |
| EPVTVSWNSGALTSGVHTFPAVLQSSG | | FPEPVTVSWNSGALTSGVHTFPAVL | |
| LYSLSSVVTVPSSSLGTQTYICNVNHKP | | QSSGLYSLSSVVTVPSSSLGTQTYIC | |
| SNTKVDKKVEPKSCDKTHTCPPCPAPE | | NVNHKPSNTKVDKKVEPKSCDKTH | |
| AAGGPSVFLFPPKPKDTLMISRTPEVTC | | TCPPCPAPEAAGGPSVFLFPPKPKDT | |
| VVVSVSHEDPEVKFNWYVDGVEVHN | | LMISRTPEVTCVVVSVSHEDPEVKF | |
| AKTKPREEQYNSTYRVVSVLTVLHQD | | NWYVDGVEVHNAKTKPREEQYNS | |
| WLNGKEYKCKVSNKALPAPIEKTISKA | | TYRVVSVLTVLHQDWLNGKEYKCK | |
| KGQPREPQVYTLPPSRDELTKNQVSLS | | VSNKALPAPIEKTISKAKGQPREPQV | |
| CAVKGFYPSDIAVEWESNGQPENNYKT | | YTLPPSRDELTKNQVSLWCLVKGFY | |
| TPPVLDSDGSFFLVSKLTVDKSRWQQG | | PSDIAVEWESNGQPENNYKTTPPVL | |
| NVFSCSVMHEALHNHYTQKSLSLSPG | | DSDGSFFLYSKLTVDKSRWQQGNVF | |
| K | | SCSVMHEALHNHYTQKSLSLSPGK | |

| | |
|---|---|
| cLC: | SEQ ID |
| DIQMTQSPSSLSASVGDRVTITCRASQSISSYLNWYQQKPGKAPKLLIYAASSLQSGVPSRFS | NO: 37 |
| GSGSGTDFTLTISSLQPEDFATYYCQQSYSTPPTFGQGTKVEIKRTVAAPSVFIFPPSDEQLKS | |
| GTASVVCLLNNFYPREAKVQWKVDNALQSGNSQESVTEQDSKDSTYSLSSTLTLSKADYE | |
| KHKVYACEVTHQGLSSPVTKSFNRGEC | |

TCE 2

| HUMAN hFGFR3IIIb S249C CLONE A | | HUMAN CD3 CLONE B | |
|---|---|---|---|
| HCDR1: | SEQ ID | HCDR4: | SEQ ID |
| TFSGFSLSTSGEGVG | NO: 1 | KASGGTFRSFGIS | NO: 16 |
| HCDR2: | SEQ ID | HCDR5: | SEQ ID |
| LIYWTDDKR | NO: 2 | GFIPVLGTAN | NO: 17 |
| HCDR3: | SEQ ID | HCDR6: | SEQ ID |
| AHRRGGYSDHDSYYYYYLDV | NO: 3 | ARRGNWNPFDP | NO: 18 |
| LCDR1: | SEQ ID | LCDR4: | SEQ ID |
| RASQSISSYLN | NO: 4 | RASQSISSYLN | NO: 4 |
| LCDR2: | SEQ ID | LCDR5: | SEQ ID |
| YAASSLQS | NO: 5 | YAASSLQS | NO: 5 |
| LCDR3: | SEQ ID | LCDR6: | SEQ ID |
| QQSYSTPPT | NO: 6 | QQSYSTPPT | NO: 6 |
| VH1: | SEQ ID | VH2: | SEQ ID |
| QITLKESGPTLVKPTQTLTLTCTFSGFS | NO: 22 | EVQLVQSGAEVKKPGSSVKVSCKAS | NO: 28 |
| LSTSGEGVGWIRQPPGKALEWLALIY | | GGTFRSFGISWVRQAPGQGLEWMG | |
| WTDDKRYSPSLKSRLTITKDTSKNHVV | | GFIPVLGTANYAQKFQGRVTIIADKS | |
| LTMTNMDPVDTATYYCAHRRGGYSD | | TNTAYMELSSLRSEDTAVYYCARRG | |
| HDSYYYYYLDVWGKGTTVTVSS | | NWNPFDPWGQGTLVTVSS | |
| VL1 and 2: | | | SEQ ID |
| DIQMTQSPSSLSASVGDRVTITCRASQSISSYLNWYQQKPGKAPKLLIYAASSLQSGVPSRFS | | | NO: 23 |
| GSGSGTDFTLTISSLQPEDFATYYCQQSYSTPPTFGQGTKVEIK | | | |
| Constant Heavy (CH): | SEQ ID | CH2: | SEQ ID |
| ASTKGPSVFPLAPSSKSTSGGTAALGC | NO: 44 | ASTKGPSVFPLAPSSKSTSGGTAALG | NO: 30 |
| LVKDYFPEPVTVSWNSGALTSGVHTFP | | CLVKDYFPEPVTVSWNSGALTSGVH | |
| AVLQSSGLYSLSSVVTVPSSSLGTQTYI | | TFPAVLQSSGLYSLSSVVTVPSSSLGT | |
| CNVNHKPSNTKVDKKVEPKSCDKTHT | | QTYICNVNHKPSNTKVDKKVEPKSC | |
| CPPCPAPEAAGGPSVFLFPPKPKDTLMI | | DKTHTCPPCPAPEAAGGPSVFLFPPK | |
| SRTPEVTCVVVSVSHEDPEVKFNWYV | | PKDTLMISRTPEVTCVVVSVSHEDPE | |
| DGVEVHNAKTKPREEQYNSTYRVVSV | | VKFNWYVDGVEVHNAKTKPREEQ | |
| LTVLHQDWLNGKEYKCKVSNKALPAP | | YNSTYRVVSVLTVLHQDWLNGKEY | |
| IEKTISKAKGQPREPQVYTLPPSRDELT | | KCKVSNKALPAPIEKTISKAKGQPRE | |
| KNQVSLSCAVKGFYPSDIAVEWESNG | | PQVYTLPPSRDELTKNQVSLWCLVK | |
| QPENNYKTTPPVLDSDGSFFLVSKLTV | | GFYPSDIAVEWESNGQPENNYKTTP | |
| DKSRWQQGNVFSCSVMHEALHNHYT | | PVLDSDGSFFLYSKLTVDKSRWQQG | |
| QKSLSLSPGK | | NVFSCSVMHEALHNHYTQKSLSLSP | |
| | | GK | |
| Constant Light (CL): | | | SEQ ID |
| RTVAAPSVFIFPPSDEQLKSGTASVVCLLNNFYPREAKVQWKVDNALQSGNSQESVTEQD | | | NO: 31 |
| SKDSTYSLSSTLTLSKADYEKHKVYACEVTHQGLSSPVTKSFNRGEC | | | |
| HC1: | SEQ ID | HC2: | SEQ ID |
| QITLKESGPTLVKPTQTLTLTCTFSGFSL | NO: 32 | EVQLVQSGAEVKKPGSSVKVSCKAS | NO: 39 |
| STSGEGVGWIRQPPGKALEWLALIYW | | GGTFRSFGISWVRQAPGQGLEWMG | |
| TDDKRYSPSLKSRLTITKDTSKNHVVLT | | GFIPVLGTANYAQKFQGRVTIIADKS | |
| MTNMDPVDTATYYCAHRRGGYSDHD | | TNTAYMELSSLRSEDTAVYYCARRG | |
| SYYYYYLDVWGKGTTVTVSSASTKGP | | NWNPFDPWGQGTLVTVSSASTKGP | |
| SVFPLAPSSKSTSGGTAALGCLVKDYFP | | SVFPLAPSSKSTSGGTAALGCLVKD | |
| EPVTVSWNSGALTSGVHTFPAVLQSSG | | YFPEPVTVSWNSGALTSGVHTFPAV | |
| LYSLSSVVTVPSSSLGTQTYICNVNHKP | | LQSSGLYSLSSVVTVPSSSLGTQTYI | |
| SNTKVDKKVEPKSCDKTHTCPPCPAPE | | CNVNHKPSNTKVDKKVEPKSCDKT | |
| AAGGPSVFLFPPKPKDTLMISRTPEVTC | | HTCPPCPAPEAAGGPSVFLFPPKPKD | |
| VVVSVSHEDPEVKFNWYVDGVEVHN | | TLMISRTPEVTCVVVSVSHEDPEVKF | |
| AKTKPREEQYNSTYRVVSVLTVLHQD | | NWYVDGVEVHNAKTKPREEQYNS | |
| WLNGKEYKCKVSNKALPAPIEKTISKA | | TYRVVSVLTVLHQDWLNGKEYKCK | |
| KGQPREPQVYTLPPSRDELTKNQVSLS | | VSNKALPAPIEKTISKAKGQPREPQV | |
| CAVKGFYPSDIAVEWESNGQPENNYKT | | YTLPPSRDELTKNQVSLWCLVKGFY | |
| TPPVLDSDGSFFLVSKLTVDKSRWQQG | | PSDIAVEWESNGQPENNYKTTPPVL | |
| NVFSCSVMHEALHNHYTQKSLSLSPG | | DSDGSFFLYSKLTVDKSRWQQGNVF | |
| K | | SCSVMHEALHNHYTQKSLSLSPGK | |

| | |
|---|---|
| cLC: | SEQ ID |
| DIQMTQSPSSLSASVGDRVTITCRASQSISSYLNWYQQKPGKAPKLLIYAASSLQSGVPSRFS | NO: 37 |
| GSGSGTDFTLTISSLQPEDFATYYCQQSYSTPPTFGQGTKVEIKRTVAAPSVFIFPPSDEQLKS | |
| GTASVVCLLNNFYPREAKVQWKVDNALQSGNSQESVTEQDSKDSTYSLSSTLTLSKADYE | |
| KHKVYACEVTHQGLSSPVTKSFNRGEC | |

TCE 3

| HUMAN hFGFR3IIIb S249C CLONE B | | HUMAN CD3 CLONE C | |
|---|---|---|---|
| HCDR1: | SEQ ID | HCDR4: | SEQ ID |
| TFSGFSLSTSGEGVG | NO: 1 | ATSGFTFDDYTMH | NO: 19 |
| HCDR2: | SEQ ID | HCDR5: | SEQ ID |
| LIYWSDDKR | NO: 7 | ISWSSGSIG | NO: 20 |
| HCDR3: | SEQ ID | HCDR6: | SEQ ID |
| AHRRGGYSDHDSYYYYYMDV | NO: 8 | AKDHRGYGDYEGGGFDH | NO: 21 |
| LCDR1: | SEQ ID | LCDR4: | SEQ ID |
| RASQSISSYLN | NO: 4 | RASQSISSYLN | NO: 4 |
| LCDR2: | SEQ ID | LCDR5: | SEQ ID |
| YAASSLQS | NO: 5 | YAASSLQS | NO: 5 |
| LCDR3: | SEQ ID | LCDR6: | SEQ ID |
| QQSYSTPPT | NO: 6 | QQSYSTPPT | NO: 6 |
| VH1: | SEQ ID | VH2: | SEQ ID |
| QITLKESGPTLVKPTQTLTLTCTFSGFS | NO: 24 | EVQLVESGGGLVQPGRSLRLSCATS | NO: 45 |
| LSTSGEGVGWIRQPPGKALEWLALIY | | GFTFDDYTMHWVRQAPGKGLEWV | |
| WSDDKRYSPSLKNRLTITKDTSKNQV | | SDISWSSGSIGYADSVKGRFTISRDN | |
| VLTMTNMDPVDTATYYCAHRRGGYS | | AKNSLYLQMNSLRAEDTALYFCAK | |
| DHDSYYYYYMDVWGKGTMVTVSS | | DHRGYGDYEGGGFDHWGQGTLVT | |
| | | VSS | |
| VL1 and 2: | | | SEQ ID |
| DIQMTQSPSSLSASVGDRVTITCRASQSISSYLNWYQQKPGKAPKLLIYAASSLQSGVPSRFS | | | NO: 23 |
| GSGSGTDFTLTISSLQPEDFATYYCQQSYSTPPTFGQGTKVEIK | | | |
| Constant Heavy (CH): | SEQ ID | CH2: | SEQ ID |
| ASTKGPSVFPLAPSSKSTSGGTAALGC | NO: 44 | ASTKGPSVFPLAPSSKSTSGGTAALG | NO: 30 |
| LVKDYFPEPVTVSWNSGALTSGVHTFP | | CLVKDYFPEPVTVSWNSGALTSGVH | |
| AVLQSSGLYSLSSVVTVPSSSLGTQTYI | | TFPAVLQSSGLYSLSSVVTVPSSSLGT | |
| CNVNHKPSNTKVDKKVEPKSCDKTHT | | QTYICNVNHKPSNTKVDKKVEPKSC | |
| CPPCPAPEAAGGPSVFLFPPKPKDTLMI | | DKTHTCPPCPAPEAAGGPSVFLFPPK | |
| SRTPEVTCVVVSVSHEDPEVKFNWYV | | PKDTLMISRTPEVTCVVVSVSHEDPE | |
| DGVEVHNAKTKPREEQYNSTYRVVSV | | VKFNWYVDGVEVHNAKTKPREEQ | |
| LTVLHQDWLNGKEYKCKVSNKALPAP | | YNSTYRVVSVLTVLHQDWLNGKEY | |
| IEKTISKAKGQPREPQVYTLPPSRDELT | | KCKVSNKALPAPIEKTISKAKGQPRE | |
| KNQVSLSCAVKGFYPSDIAVEWESNG | | PQVYTLPPSRDELTKNQVSLWCLVK | |
| QPENNYKTTPPVLDSDGSFFLVSKLTV | | GFYPSDIAVEWESNGQPENNYKTTP | |
| DKSRWQQGNVFSCSVMHEALHNHYT | | PVLDSDGSFFLYSKLTVDKSRWQQG | |
| QKSLSLSPGK | | NVFSCSVMHEALHNHYTQKSLSLSP | |
| | | GK | |
| Constant Light (CL): | | | SEQ ID |
| RTVAAPSVFIFPPSDEQLKSGTASVVCLLNNFYPREAKVQWKVDNALQSGNSQESVTEQD | | | NO: 31 |
| SKDSTYSLSSTLTLSKADYEKHKVYACEVTHQGLSSPVTKSFNRGEC | | | |
| HC1: | SEQ ID | HC2: | SEQ ID |
| QITLKESGPTLVKPTQTLTLTCTFSGFS | NO: 34 | EVQLVESGGGLVQPGRSLRLSCATS | NO: 38 |
| LSTSGEGVGWIRQPPGKALEWLALIY | | GFTFDDYTMHWVRQAPGKGLEWV | |
| WSDDKRYSPSLKNRLTITKDTSKNQV | | SDISWSSGSIGYADSVKGRFTISRDN | |
| VLTMTNMDPVDTATYYCAHRRGGYS | | AKNSLYLQMNSLRAEDTALYFCAK | |
| DHDSYYYYYMDVWGKGTMVTVSSAS | | DHRGYGDYEGGGFDHWGQGTLVT | |
| TKGPSVFPLAPSSKSTSGGTAALGCLV | | VSSASTKGPSVFPLAPSSKSTSGGTA | |
| KDYFPEPVTVSWNSGALTSGVHTFPA | | ALGCLVKDYFPEPVTVSWNSGALTS | |
| VLQSSGLYSLSSVVTVPSSSLGTQTYIC | | GVHTFPAVLQSSGLYSLSSVVTVPSS | |
| NVNHKPSNTKVDKKVEPKSCDKTHTC | | SLGTQTYICNVNHKPSNTKVDKKVE | |
| PPCPAPEAAGGPSVFLFPPKPKDTLMIS | | PKSCDKTHTCPPCPAPEAAGGPSVFL | |
| RTPEVTCVVVSVSHEDPEVKFNWYVD | | FPPKPKDTLMISRTPEVTCVVVSVSH | |
| GVEVHNAKTKPREEQYNSTYRVVSVL | | EDPEVKFNWYVDGVEVHNAKTKPR | |
| TVLHQDWLNGKEYKCKVSNKALPAPI | | EEQYNSTYRVVSVLTVLHQDWLNG | |
| EKTISKAKGQPREPQVYTLPPSRDELT | | KEYKCKVSNKALPAPIEKTISKAKG | |
| KNQVSLSCAVKGFYPSDIAVEWESNG | | QPREPQVYTLPPSRDELTKNQVSLW | |
| QPENNYKTTPPVLDSDGSFFLVSKLTV | | CLVKGFYPSDIAVEWESNGQPENNY | |
| DKSRWQQGNVFSCSVMHEALHNHYT | | KTTPPVLDSDGSFFLYSKLTVDKSR | |
| QKSLSLSPGK | | WQQGNVFSCSVMHEALHNHYTQK | |
| | | SLSLSPGK | |

| | |
|---|---|
| CLC: | SEQ ID |
| DIQMTQSPSSLSASVGDRVTITCRASQSISSYLNWYQQKPGKAPKLLIYAASSLQSGVPSRFS | NO: 37 |
| GSGSGTDFTLTISSLQPEDFATYYCQQSYSTPPTFGQGTKVEIKRTVAAPSVFIFPPSDEQLKS | |
| GTASVVCLLNNFYPREAKVQWKVDNALQSGNSQESVTEQDSKDSTYSLSSTLTLSKADYE | |
| KHKVYACEVTHQGLSSPVTKSFNRGEC | |

TCE 4

| HUMAN hFGFR3IIIb S249C CLONE C | | HUMAN CD3 CLONE B | |
|---|---|---|---|
| HCDR1: | SEQ ID | HCDR4: | SEQ ID |
| TFSGFSLSTSGEAVG | NO: 9 | KASGGTFRSFGIS | NO: 16 |
| HCDR2: | SEQ ID | HCDR5: | SEQ ID |
| LIYWSDDSR | NO: 10 | GFIPVLGTAN | NO: 17 |
| HCDR3: | SEQ ID | HCDR6: | SEQ ID |
| AHRRGSYSGDYYYYYMDV | NO: 11 | ARRGNWNPFDP | NO: 18 |
| LCDR1: | SEQ ID | LCDR4: | SEQ ID |
| RASQSISSYLN | NO: 4 | RASQSISSYLN | NO: 4 |
| LCDR2: | SEQ ID | LCDR5: | SEQ ID |
| YAASSLQS | NO: 5 | YAASSLQS | NO: 5 |
| LCDR3: | SEQ ID | LCDR6: | SEQ ID |
| QQSYSTPPT | NO: 6 | QQSYSTPPT | NO: 6 |
| VH1: | SEQ ID | VH2: | SEQ ID |
| QITLKESGPTLVKPTQTLTLTCTFSGFS | NO: 25 | EVQLVQSGAEVKKPGSSVKVSCKAS | NO: 28 |
| LSTSGEAVGWIRQPPGKALEWLALIY | | GGTFRSFGISWVRQAPGQGLEWMG | |
| WSDDSRYSPSLKSRLTITRVTAKNQVV | | GFIPVLGTANYAQKFQGRVTIIADKS | |
| LKMTNMDPVDTATYYCAHRRGSYSG | | TNTAYMELSSLRSEDTAVYYCARRG | |
| DYYYYYMDVWGKGTMVTVSS | | NWNPFDPWGQGTLVTVSS | |
| VL1 and 2: | | | SEQ ID |
| DIQMTQSPSSLSASVGDRVTITCRASQSISSYLNWYQQKPGKAPKLLIYAASSLQSGVPSRFS | | | NO: 23 |
| GSGSGTDFTLTISSLQPEDFATYYCQQSYSTPPTFGQGTKVEIK | | | |
| Constant Heavy (CH): | SEQ ID | CH2: | SEQ ID |
| ASTKGPSVFPLAPSSKSTSGGTAALGC | NO: 44 | ASTKGPSVFPLAPSSKSTSGGTAALG | NO: 30 |
| LVKDYFPEPVTVSWNSGALTSGVHTFP | | CLVKDYFPEPVTVSWNSGALTSGVH | |
| AVLQSSGLYSLSSVVTVPSSSLGTQTYI | | TFPAVLQSSGLYSLSSVVTVPSSSLGT | |
| CNVNHKPSNTKVDKKVEPKSCDKTHT | | QTYICNVNHKPSNTKVDKKVEPKSC | |
| CPPCPAPEAAGGPSVFLFPPKPKDTLMI | | DKTHTCPPCPAPEAAGGPSVFLFPPK | |
| SRTPEVTCVVVSVSHEDPEVKFNWYV | | PKDTLMISRTPEVTCVVVSVSHEDPE | |
| DGVEVHNAKTKPREEQYNSTYRVVSV | | VKFNWYVDGVEVHNAKTKPREEQ | |
| LTVLHQDWLNGKEYKCKVSNKALPAP | | YNSTYRVVSVLTVLHQDWLNGKEY | |
| IEKTISKAKGQPREPQVYTLPPSRDELT | | KCKVSNKALPAPIEKTISKAKGQPRE | |
| KNQVSLSCAVKGFYPSDIAVEWESNG | | PQVYTLPPSRDELTKNQVSLWCLVK | |
| QPENNYKTTPPVLDSDGSFFLVSKLTV | | GFYPSDIAVEWESNGQPENNYKTTP | |
| DKSRWQQGNVFSCSVMHEALHNHYT | | PVLDSDGSFFLYSKLTVDKSRWQQG | |
| QKSLSLSPGK | | NVFSCSVMHEALHNHYTQKSLSLSP | |
| | | GK | |
| Constant Light (CL): | | | SEQ ID |
| RTVAAPSVFIFPPSDEQLKSGTASVVCLLNNFYPREAKVQWKVDNALQSGNSQESVTEQD | | | NO: 31 |
| SKDSTYSLSSTLTLSKADYEKHKVYACEVTHQGLSSPVTKSFNRGEC | | | |
| HC1: | SEQ ID | HC2: | SEQ ID |
| QITLKESGPTLVKPTQTLTLTCTFSGFS | NO: 35 | EVQLVQSGAEVKKPGSSVKVSCKAS | NO: 39 |
| LSTSGEAVGWIRQPPGKALEWLALIY | | GGTFRSFGISWVRQAPGQGLEWMG | |
| WSDDSRYSPSLKSRLTITRVTAKNQVV | | GFIPVLGTANYAQKFQGRVTIIADKS | |
| LKMTNMDPVDTATYYCAHRRGSYSG | | TNTAYMELSSLRSEDTAVYYCARRG | |
| DYYYYYMDVWGKGTMVTVSSASTKG | | NWNPFDPWGQGTLVTVSSASTKGP | |
| PSVFPLAPSSKSTSGGTAALGCLVKDY | | SVFPLAPSSKSTSGGTAALGCLVKD | |
| FPEPVTVSWNSGALTSGVHTFPAVLQS | | YFPEPVTVSWNSGALTSGVHTFPAV | |
| SGLYSLSSVVTVPSSSLGTQTYICNVNH | | LQSSGLYSLSSVVTVPSSSLGTQTYI | |
| KPSNTKVDKKVEPKSCDKTHTCPPCPA | | CNVNHKPSNTKVDKKVEPKSCDKT | |
| PEAAGGPSVFLFPPKPKDTLMISRTPEV | | HTCPPCPAPEAAGGPSVFLFPPKPKD | |
| TCVVVSVSHEDPEVKFNWYVDGVEV | | TLMISRTPEVTCVVVSVSHEDPEVKF | |
| HNAKTKPREEQYNSTYRVVSVLTVLH | | NWYVDGVEVHNAKTKPREEQYNS | |
| QDWLNGKEYKCKVSNKALPAPIEKTIS | | TYRVVSVLTVLHQDWLNGKEYKCK | |
| KAKGQPREPQVYTLPPSRDELTKNQVS | | VSNKALPAPIEKTISKAKGQPREPQV | |
| LSCAVKGFYPSDIAVEWESNGQPENN | | YTLPPSRDELTKNQVSLWCLVKGFY | |
| YKTTPPVLDSDGSFFLVSKLTVDKSRW | | PSDIAVEWESNGQPENNYKTTPPVL | |
| QQGNVFSCSVMHEALHNHYTQKSLSL | | DSDGSFFLYSKLTVDKSRWQQGNVF | |
| SPGK | | SCSVMHEALHNHYTQKSLSLSPGK | |

| | |
|---|---|
| CLC: | SEQ ID |
| DIQMTQSPSSLSASVGDRVTITCRASQSISSYLNWYQQKPGKAPKLLIYAASSLQSGVPSRF | NO: 37 |
| SGSGSGTDFTLTISSLQPEDFATYYCQQSYSTPPTFGQGTKVEIKRTVAAPSVFIFPPSDEQL | |
| KSGTASVVCLLNNFYPREAKVQWKVDNALQSGNSQESVTEQDSKDSTYSLSSTLTLSKAD | |
| YEKHKVYACEVTHQGLSSPVTKSFNRGEC | |

TCE 5

| HUMAN hFGFR3IIIb S249C CLONE A | | HUMAN CD3 CLONE C | |
|---|---|---|---|
| HCDR1: | SEQ ID | HCDR4: | SEQ ID |
| TFSGFSLSTSGEGVG | NO: 1 | ATSGFTFDDYTMH | NO: 19 |
| HCDR2: | SEQ ID | HCDR5: | SEQ ID |
| LIYWTDDKR | NO: 2 | ISWSSGSIG | NO: 20 |
| HCDR3: | SEQ ID | HCDR6: | SEQ ID |
| AHRRGGYSDHDSYYYYYLDV | NO: 3 | AKDHRGYGDYEGGGFDH | NO: 21 |
| LCDR1: | SEQ ID | LCDR4: | SEQ ID |
| RASQSISSYLN | NO: 4 | RASQSISSYLN | NO: 4 |
| LCDR2: | SEQ ID | LCDR5: | SEQ ID |
| YAASSLQS | NO: 5 | YAASSLQS | NO: 5 |
| LCDR3: | SEQ ID | LCDR6: | SEQ ID |
| QQSYSTPPT | NO: 6 | QQSYSTPPT | NO: 6 |
| VH1: | SEQ ID | VH2: | SEQ ID |
| QITLKESGPTLVKPTQTLTLTCTFSGFSL | NO: 22 | EVQLVESGGGLVQPGRSLRLSCATS | NO: 45 |
| STSGEGVGWIRQPPGKALEWLALIYW | | GFTFDDYTMHWVRQAPGKGLEWV | |
| TDDKRYSPSLKSRLTITKDTSKNHVVLT | | SDISWSSGSIGYADSVKGRFTISRDN | |
| MTNMDPVDTATYYCAHRRGGYSDHD | | AKNSLYLQMNSLRAEDTALYFCAK | |
| SYYYYYLDVWGKGTTVTVSS | | DHRGYGDYEGGGFDHWGQGTLVT | |
| | | VSS | |
| VL1 and 2: | | | SEQ ID |
| DIQMTQSPSSLSASVGDRVTITCRASQSISSYLNWYQQKPGKAPKLLIYAASSLQSGVPSRFS | | | NO: 23 |
| GSGSGTDFTLTISSLQPEDFATYYCQQSYSTPPTFGQGTKVEIK | | | |
| Constant Heavy (CH): | SEQ ID | CH2: | SEQ ID |
| ASTKGPSVFPLAPSSKSTSGGTAALGC | NO: 44 | ASTKGPSVFPLAPSSKSTSGGTAALG | NO: 30 |
| LVKDYFPEPVTVSWNSGALTSGVHTFP | | CLVKDYFPEPVTVSWNSGALTSGVH | |
| AVLQSSGLYSLSSVVTVPSSSLGTQTYI | | TFPAVLQSSGLYSLSSVVTVPSSSLGT | |
| CNVNHKPSNTKVDKKVEPKSCDKTHT | | QTYICNVNHKPSNTKVDKKVEPKSC | |
| CPPCPAPEAAGGPSVFLFPPKPKDTLMI | | DKTHTCPPCPAPEAAGGPSVFLFPPK | |
| SRTPEVTCVVVSVSHEDPEVKFNWYV | | PKDTLMISRTPEVTCVVVSVSHEDPE | |
| DGVEVHNAKTKPREEQYNSTYRVVSV | | VKFNWYVDGVEVHNAKTKPREEQ | |
| LTVLHQDWLNGKEYKCKVSNKALPAP | | YNSTYRVVSVLTVLHQDWLNGKEY | |
| IEKTISKAKGQPREPQVYTLPPSRDELT | | KCKVSNKALPAPIEKTISKAKGQPRE | |
| KNQVSLSCAVKGFYPSDIAVEWESNG | | PQVYTLPPSRDELTKNQVSLWCLVK | |
| QPENNYKTTPPVLDSDGSFFLVSKLTV | | GFYPSDIAVEWESNGQPENNYKTTP | |
| DKSRWQQGNVFSCSVMHEALHNHYT | | PVLDSDGSFFLYSKLTVDKSRWQQG | |
| QKSLSLSPGK | | NVFSCSVMHEALHNHYTQKSLSLSP | |
| | | GK | |
| Constant Light (CL): | | | SEQ ID |
| RTVAAPSVFIFPPSDEQLKSGTASVVCLLNNFYPREAKVQWKVDNALQSGNSQESVTEQD | | | NO: 31 |
| SKDSTYSLSSTLTLSKADYEKHKVYACEVTHQGLSSPVTKSFNRGEC | | | |
| HC1: | SEQ ID | HC2: | SEQ ID |
| QITLKESGPTLVKPTQTLTLTCTFSGFSL | NO: 32 | EVQLVESGGGLVQPGRSLRLSCATS | NO: 38 |
| STSGEGVGWIRQPPGKALEWLALIYW | | GFTFDDYTMHWVRQAPGKGLEWV | |
| TDDKRYSPSLKSRLTITKDTSKNHVVLT | | SDISWSSGSIGYADSVKGRFTISRDN | |
| MTNMDPVDTATYYCAHRRGGYSDHD | | AKNSLYLQMNSLRAEDTALYFCAK | |
| SYYYYYLDVWGKGTTVTVSSASTKGP | | DHRGYGDYEGGGFDHWGQGTLVT | |
| SVFPLAPSSKSTSGGTAALGCLVKDYFP | | VSSASTKGPSVFPLAPSSKSTSGGTA | |
| EPVTVSWNSGALTSGVHTFPAVLQSSG | | ALGCLVKDYFPEPVTVSWNSGALTS | |
| LYSLSSVVTVPSSSLGTQTYICNVNHKP | | GVHTFPAVLQSSGLYSLSSVVTVPSS | |
| SNTKVDKKVEPKSCDKTHTCPPCPAPE | | SLGTQTYICNVNHKPSNTKVDKKVE | |
| AAGGPSVFLFPPKPKDTLMISRTPEVTC | | PKSCDKTHTCPPCPAPEAAGGPSVFL | |
| VVVSVSHEDPEVKFNWYVDGVEVHN | | FPPKPKDTLMISRTPEVTCVVVSVSH | |
| AKTKPREEQYNSTYRVVSVLTVLHQD | | EDPEVKFNWYVDGVEVHNAKTKPR | |
| WLNGKEYKCKVSNKALPAPIEKTISKA | | EEQYNSTYRVVSVLTVLHQDWLNG | |
| KGQPREPQVYTLPPSRDELTKNQVSLS | | KEYKCKVSNKALPAPIEKTISKAKG | |
| CAVKGFYPSDIAVEWESNGQPENNYKT | | QPREPQVYTLPPSRDELTKNQVSLW | |
| TPPVLDSDGSFFLVSKLTVDKSRWQQG | | CLVKGFYPSDIAVEWESNGQPENNY | |
| NVFSCSVMHEALHNHYTQKSLSLSPG | | KTTPPVLDSDGSFFLYSKLTVDKSR | |
| K | | WQQGNVFSCSVMHEALHNHYTQK | |
| | | SLSLSPGK | |

| | |
|---|---|
| cLC: | SEQ ID |
| DIQMTQSPSSLSASVGDRVTITCRASQSISSYLNWYQQKPGKAPKLLIYAASSLQSGVPSRFS | NO: 37 |
| GSGSGTDFTLTISSLQPEDFATYYCQQSYSTPPTFGQGTKVEIKRTVAAPSVFIFPPSDEQLKS | |
| GTASVVCLLNNFYPREAKVQWKVDNALQSGNSQESVTEQDSKDSTYSLSSTLTLSKADYE | |
| KHKVYACEVTHQGLSSPVTKSFNRGEC | |

TCE 6

| HUMAN hFGFR3IIIb S249C CLONE C | | HUMAN CD3 CLONE C | |
|---|---|---|---|
| HCDR1: | SEQ ID | HCDR4: | SEQ ID |
| TFSGFSLSTSGEAVG | NO: 9 | ATSGFTFDDYTMH | NO: 19 |
| HCDR2: | SEQ ID | HCDR5: | SEQ ID |
| LIYWSDDSR | NO: 10 | ISWSSGSIG | NO: 20 |
| HCDR3: | SEQ ID | HCDR6: | SEQ ID |
| AHRRGSYSGDYYYYYMDV | NO: 11 | AKDHRGYGDYEGGGFDH | NO: 21 |
| LCDR1: | SEQ ID | LCDR4: | SEQ ID |
| RASQSISSYLN | NO: 4 | RASQSISSYLN | NO: 4 |
| LCDR2: | SEQ ID | LCDR5: | SEQ ID |
| YAASSLQS | NO: 5 | YAASSLQS | NO: 5 |
| LCDR3: | SEQ ID | LCDR6: | SEQ ID |
| QQSYSTPPT | NO: 6 | QQSYSTPPT | NO: 6 |
| VH1: | SEQ ID | VH2: | SEQ ID |
| QITLKESGPTLVKPTQTLTLTCTFSGFS | NO: 25 | EVQLVESGGGLVQPGRSLRLSCATS | NO: 45 |
| LSTSGEAVGWIRQPPGKALEWLALIY | | GFTFDDYTMHWVRQAPGKGLEWV | |
| WSDDSRYSPSLKSRLTITRVTAKNQVV | | SDISWSSGSIGYADSVKGRFTISRDN | |
| LKMTNMDPVDTATYYCAHRRGSYSG | | AKNSLYLQMNSLRAEDTALYFCAK | |
| DYYYYYMDVWGKGTMVTVSS | | DHRGYGDYEGGGFDHWGQGTLVT | |
| | | VSS | |
| VL1 and 2: | | | SEQ ID |
| DIQMTQSPSSLSASVGDRVTITCRASQSISSYLNWYQQKPGKAPKLLIYAASSLQSGVPSRFS | | | NO: 23 |
| GSGSGTDFTLTISSLQPEDFATYYCQQSYSTPPTFGQGTKVEIK | | | |
| Constant Heavy (CH): | SEQ ID | CH2: | SEQ ID |
| ASTKGPSVFPLAPSSKSTSGGTAALGC | NO: 44 | ASTKGPSVFPLAPSSKSTSGGTAALG | NO: 30 |
| LVKDYFPEPVTVSWNSGALTSGVHTFP | | CLVKDYFPEPVTVSWNSGALTSGVH | |
| AVLQSSGLYSLSSVVTVPSSSLGTQTYI | | TFPAVLQSSGLYSLSSVVTVPSSSLGT | |
| CNVNHKPSNTKVDKKVEPKSCDKTHT | | QTYICNVNHKPSNTKVDKKVEPKSC | |
| CPPCPAPEAAGGPSVFLFPPKPKDTLMI | | DKTHTCPPCPAPEAAGGPSVFLFPPK | |
| SRTPEVTCVVVSVSHEDPEVKFNWYV | | PKDTLMISRTPEVTCVVVSVSHEDPE | |
| DGVEVHNAKTKPREEQYNSTYRVVSV | | VKFNWYVDGVEVHNAKTKPREEQ | |
| LTVLHQDWLNGKEYKCKVSNKALPAP | | YNSTYRVVSVLTVLHQDWLNGKEY | |
| IEKTISKAKGQPREPQVYTLPPSRDELT | | KCKVSNKALPAPIEKTISKAKGQPRE | |
| KNQVSLSCAVKGFYPSDIAVEWESNG | | PQVYTLPPSRDELTKNQVSLWCLVK | |
| QPENNYKTTPPVLDSDGSFFLVSKLTV | | GFYPSDIAVEWESNGQPENNYKTTP | |
| DKSRWQQGNVFSCSVMHEALHNHYT | | PVLDSDGSFFLYSKLTVDKSRWQQG | |
| QKSLSLSPGK | | NVFSCSVMHEALHNHYTQKSLSLSP | |
| | | GK | |
| Constant Light (CL): | | | SEQ ID |
| RTVAAPSVFIFPPSDEQLKSGTASVVCLLNNFYPREAKVQWKVDNALQSGNSQESVTEQD | | | NO: 31 |
| SKDSTYSLSSTLTLSKADYEKHKVYACEVTHQGLSSPVTKSFNRGEC | | | |
| HC1: | SEQ ID | HC2: | SEQ ID |
| QITLKESGPTLVKPTQTLTLTCTFSGFS | NO: 35 | EVQLVESGGGLVQPGRSLRLSCATS | NO: 38 |
| LSTSGEAVGWIRQPPGKALEWLALIY | | GFTFDDYTMHWVRQAPGKGLEWV | |
| WSDDSRYSPSLKSRLTITRVTAKNQVV | | SDISWSSGSIGYADSVKGRFTISRDN | |
| LKMTNMDPVDTATYYCAHRRGSYSG | | AKNSLYLQMNSLRAEDTALYFCAK | |
| DYYYYYMDVWGKGTMVTVSSASTKG | | DHRGYGDYEGGGFDHWGQGTLVT | |
| PSVFPLAPSSKSTSGGTAALGCLVKDY | | VSSASTKGPSVFPLAPSSKSTSGGTA | |
| FPEPVTVSWNSGALTSGVHTFPAVLQS | | ALGCLVKDYFPEPVTVSWNSGALTS | |
| SGLYSLSSVVTVPSSSLGTQTYICNVNH | | GVHTFPAVLQSSGLYSLSSVVTVPSS | |
| KPSNTKVDKKVEPKSCDKTHTCPPCPA | | SLGTQTYICNVNHKPSNTKVDKKVE | |
| PEAAGGPSVFLFPPKPKDTLMISRTPEV | | PKSCDKTHTCPPCPAPEAAGGPSVFL | |
| TCVVVSVSHEDPEVKFNWYVDGVEV | | FPPKPKDTLMISRTPEVTCVVVSVSH | |
| HNAKTKPREEQYNSTYRVVSVLTVLH | | EDPEVKFNWYVDGVEVHNAKTKPR | |
| QDWLNGKEYKCKVSNKALPAPIEKTIS | | EEQYNSTYRVVSVLTVLHQDWLNG | |
| KAKGQPREPQVYTLPPSRDELTKNQVS | | KEYKCKVSNKALPAPIEKTISKAKG | |
| LSCAVKGFYPSDIAVEWESNGQPENN | | QPREPQVYTLPPSRDELTKNQVSLW | |
| YKTTPPVLDSDGSFFLVSKLTVDKSRW | | CLVKGFYPSDIAVEWESNGQPENNY | |
| QQGNVFSCSVMHEALHNHYTQKSLSL | | KTTPPVLDSDGSFFLYSKLTVDKSR | |
| SPGK | | WQQGNVFSCSVMHEALHNHYTQK | |
| | | SLSLSPGK | |

| | |
|---|---|
| cLC: | SEQ ID |
| DIQMTQSPSSLSASVGDRVTITCRASQSISSYLNWYQQKPGKAPKLLIYAASSLQSGVPSRFS | NO: 37 |
| GSGSGTDFTLTISSLQPEDFATYYCQQSYSTPPTFGQGTKVEIKRTVAAPSVFIFPPSDEQLKS | |
| GTASVVCLLNNFYPREAKVQWKVDNALQSGNSQESVTEQDSKDSTYSLSSTLTLSKADYE | |
| KHKVYACEVTHQGLSSPVTKSFNRGEC | |

TCE 7

| HUMAN hFGFR3IIIb S249C CLONE D | | HUMAN CD3 CLONE C | |
|---|---|---|---|
| HCDR1: | SEQ ID | HCDR4: | SEQ ID |
| TFSGFSLSTSGEGVG | NO: 1 | ATSGFTFDDYTMH | NO: 19 |
| HCDR2: | SEQ ID | HCDR5: | SEQ ID |
| LIYWSDDKR | NO: 7 | ISWSSGSIG | NO: 20 |
| HCDR3: | SEQ ID | HCDR6: | SEQ ID |
| AHRRGGYSGYDSYYYYYMDV | NO: 12 | AKDHRGYGDYEGGGFDH | NO: 21 |
| LCDR1: | SEQ ID | LCDR4: | SEQ ID |
| RASQSISSYLN | NO: 4 | RASQSISSYLN | NO: 4 |
| LCDR2: | SEQ ID | LCDR5: | SEQ ID |
| YAASSLQS | NO: 5 | YAASSLQS | NO: 5 |
| LCDR3: | SEQ ID | LCDR6: | SEQ ID |
| QQSYSTPPT | NO: 6 | QQSYSTPPT | NO: 6 |
| VH1: | SEQ ID | VH2: | SEQ ID |
| RITLKESGPTLVKPTQTLTLTCTFSGFSL | NO: 26 | EVQLVESGGGLVQPGRSLRLSCATS | NO: 45 |
| STSGEGVGWIRQPPGKALEWLALIYW | | GFTFDDYTMHWVRQAPGKGLEWV | |
| SDDKRYSPSLKNRLTITKDTSKNQVVL | | SDISWSSGSIGYADSVKGRFTISRDN | |
| TMTNMDPVDTATYYCAHRRGGYSGY | | AKNSLYLQMNSLRAEDTALYFCAK | |
| DSYYYYYMDVWGKGTMVTVSS | | DHRGYGDYEGGGFDHWGQGTLVT | |
| | | VSS | |
| VL1 and 2: | | | SEQ ID |
| DIQMTQSPSSLSASVGDRVTITCRASQSISSYLNWYQQKPGKAPKLLIYAASSLQSGVPSRFS | | | NO: 23 |
| GSGSGTDFTLTISSLQPEDFATYYCQQSYSTPPTFGQGTKVEIK | | | |
| Constant Heavy (CH): | SEQ ID | CH2: | SEQ ID |
| ASTKGPSVFPLAPSSKSTSGGTAALGC | NO: 44 | ASTKGPSVFPLAPSSKSTSGGTAALG | NO: 30 |
| LVKDYFPEPVTVSWNSGALTSGVHTFP | | CLVKDYFPEPVTVSWNSGALTSGVH | |
| AVLQSSGLYSLSSVVTVPSSSLGTQTYI | | TFPAVLQSSGLYSLSSVVTVPSSSLGT | |
| CNVNHKPSNTKVDKKVEPKSCDKTHT | | QTYICNVNHKPSNTKVDKKVEPKSC | |
| CPPCPAPEAAGGPSVFLFPPKPKDTLMI | | DKTHTCPPCPAPEAAGGPSVFLFPPK | |
| SRTPEVTCVVVSVSHEDPEVKFNWYV | | PKDTLMISRTPEVTCVVVSVSHEDPE | |
| DGVEVHNAKTKPREEQYNSTYRVVSV | | VKFNWYVDGVEVHNAKTKPREEQ | |
| LTVLHQDWLNGKEYKCKVSNKALPAP | | YNSTYRVVSVLTVLHQDWLNGKEY | |
| IEKTISKAKGQPREPQVYTLPPSRDELT | | KCKVSNKALPAPIEKTISKAKGQPRE | |
| KNQVSLSCAVKGFYPSDIAVEWESNG | | PQVYTLPPSRDELTKNQVSLWCLVK | |
| QPENNYKTTPPVLDSDGSFFLVSKLTV | | GFYPSDIAVEWESNGQPENNYKTTP | |
| DKSRWQQGNVFSCSVMHEALHNHYT | | PVLDSDGSFFLYSKLTVDKSRWQQG | |
| QKSLSLSPGK | | NVFSCSVMHEALHNHYTQKSLSLSP | |
| | | GK | |
| Constant Light (CL): | | | SEQ ID |
| RTVAAPSVFIFPPSDEQLKSGTASVVCLLNNFYPREAKVQWKVDNALQSGNSQESVTEQD | | | NO: 31 |
| SKDSTYSLSSTLTLSKADYEKHKVYACEVTHQGLSSPVTKSFNRGEC | | | |
| HC1: | SEQ ID | HC2: | SEQ ID |
| RITLKESGPTLVKPTQTLTLTCTFSGFSL | NO: 36 | EVQLVESGGGLVQPGRSLRLSCATS | NO: 38 |
| STSGEGVGWIRQPPGKALEWLALIYW | | GFTFDDYTMHWVRQAPGKGLEWV | |
| SDDKRYSPSLKNRLTITKDTSKNQVVL | | SDISWSSGSIGYADSVKGRFTISRDN | |
| TMTNMDPVDTATYYCAHRRGGYSGY | | AKNSLYLQMNSLRAEDTALYFCAK | |
| DSYYYYYMDVWGKGTMVTVSSASTK | | DHRGYGDYEGGGFDHWGQGTLVT | |
| GPSVFPLAPSSKSTSGGTAALGCLVKD | | VSSASTKGPSVFPLAPSSKSTSGGTA | |
| YFPEPVTVSWNSGALTSGVHTFPAVLQ | | ALGCLVKDYFPEPVTVSWNSGALTS | |
| SSGLYSLSSVVTVPSSSLGTQTYICNVN | | GVHTFPAVLQSSGLYSLSSVVTVPSS | |
| HKPSNTKVDKKVEPKSCDKTHTCPPCP | | SLGTQTYICNVNHKPSNTKVDKKVE | |
| APEAAGGPSVFLFPPKPKDTLMISRTPE | | PKSCDKTHTCPPCPAPEAAGGPSVFL | |
| VTCVVVSVSHEDPEVKFNWYVDGVE | | FPPKPKDTLMISRTPEVTCVVVSVSH | |
| VHNAKTKPREEQYNSTYRVVSVLTVL | | EDPEVKFNWYVDGVEVHNAKTKPR | |
| HQDWLNGKEYKCKVSNKALPAPIEKTI | | EEQYNSTYRVVSVLTVLHQDWLNG | |
| SKAKGQPREPQVYTLPPSRDELTKNQV | | KEYKCKVSNKALPAPIEKTISKAKG | |
| SLSCAVKGFYPSDIAVEWESNGQPENN | | QPREPQVYTLPPSRDELTKNQVSLW | |
| YKTTPPVLDSDGSFFLVSKLTVDKSRW | | CLVKGFYPSDIAVEWESNGQPENNY | |
| QQGNVFSCSVMHEALHNHYTQKSLSL | | KTTPPVLDSDGSFFLYSKLTVDKSR | |
| SPGK | | WQQGNVFSCSVMHEALHNHYTQK | |
| | | SLSLSPGK | |

| | |
|---|---|
| CLC: | SEQ ID |
| DIQMTQSPSSLSASVGDRVTITCRASQSISSYLNWYQQKPGKAPKLLIYAASSLQSGVPSRFS | NO: 37 |
| GSGSGTDFTLTISSLQPEDFATYYCQQSYSTPPTFGQGTKVEIKRTVAAPSVFIFPPSDEQLKS | |
| GTASVVCLLNNFYPREAKVQWKVDNALQSGNSQESVTEQDSKDSTYSLSSTLTLSKADYE | |
| KHKVYACEVTHQGLSSPVTKSFNRGEC | |

### EXAMPLES

### Example 1: Generation of hFGFR3IIIb S249C×hCD3 Bispecific Antibodies

### Immunization of MeMo® Mice

A diverse panel of cLC-based hFGFR3 isoform IIIb S249C-targeting monoclonal antibodies was generated by selecting and characterizing antigen-specific molecules from MeMo® mice, which generate single light chain or common light chain (cLC) fully human antibodies in combination with diversified heavy chains (see WO2009/157771). MeMo® mice were immunized with hFGFR3 S249C antigenic moieties, including the use of different forms of DNA and cell-based delivery, as appropriate.

A diverse panel of cLC-based human CD3 monoclonal antibodies was obtained using MeMo® mice (see for instance WO2020/204708). MeMo® mice were immunized with hCD3 antigenic moieties, including the use of different forms of DNA, protein and cell-based delivery, as appropriate.

The hFGFR3 S249C and hCD3 binding domain sequences herein, once characterized and sequenced through the techniques provided herein, can be subsequently obtained by any method known in the art.

Production and Screening of hFGFR3IIIb S249C×hCD3 Bispecifics

From phage libraries generated, 34 unique hFGFR3 S249C clones were selected as being specific for the S249C mutation but not to wild-type (WT) FGFR3. Of those, the specific binding to S249C versus WT was confirmed in FACS on HeLa cells expressing FGFR3IIIb S249C vs WT (both under a CMV promotor) and on recombinant proteins in ELISA and SPR, and absence of off-target binding to FGFR1, FGFR3, or FGFR4, was verified, resulting in 21 hFGFR3IIIb S249C arms. Separately, 22 hCD3 arms were chosen from a panel having a range in affinities (from approximately 20 nM Kp to approximately over 500 nM affinity Kp as measured by surface plasmon resonance (SPR)) and hCD3 binding epitopes. The 21 hFGFR3IIIb S249C arms were cloned into an expression vector that results in L351D and L368E variations in the C_{H}3 region of the hFGFR3IIIb S249C antibody, while the 22 hCD3 arms were cloned into an expression vector that results in L351K and T366K variations in the C_{H}3 region of the hCD3 antibody, thereby allowing the two arms to efficiently heterodimerize, otherwise known as a “DEKK” backbone, according to Kabat numbering system. The hFGFR3IIIb S249C arms were paired in different combinations with the hCD3 arms, which resulted in a total of 418 bispecifics.

The resulting 418 hFGFR3IIIb S249C×hCD3 bispecifics were screened using (a) a surface plasmon resonance (SPR) analysis measuring affinity of the anti-FGFR3IIIb S249C arms for binding to human FGFR3IIIb S249C but not to wildtype FGFR3 (WT), (b) activity in T cell activation reporter assay; (c) binding titration to Hela cells expressing FGFR3IIIb S249C versus WT, and (d) developability assessment of the anti-FGFR3IIIb S249C arms. As a result, 51 hFGFR3IIIb S249C×hCD3 bispecifics were selected. Subsequent screenings were performed: cytotoxicity and cytokine release assays comparing (a) Hela cells transfected with FGFR3IIIb S249C versus WT, and then (b) on UMUC-14 cells. Finally, accelerated stability studies on the remaining bispecifics, a total of 12, were performed, resulting in 6 selected.

Reformatting of hFGFR3IIIb S249C×hCD3 Bispecifics

The 6 hFGFR3IIIb S249C×hCD3 bispecifics were reformatted from a DEKK backbone to a knob-in-hole (KiH) backbone comprising the T366W substitution in the hCD3 arm (the “knob”) and the T366S/L368A/Y407V in the hFGFR3IIIb S249C arm (the “hole”), according to Kabat numbering system. Fc silencing mutations LALADS: L234A/L235A/D265S were also introduced by conventional cloning techniques, according to Kabat numbering system. Binding affinities (K_{D}) for the six reformatted bispecifics as measured by SPR for the FGFR3IIIb S249C arm ranged from approximately 2 nM to 45 nM and for the CD3 arm ranged from approximately 20 nM to approximately 540 nM. Amino acid sequences of the TCEs of the invention are represented in Table 2 herein.

For many of the examples, the following TCEs and controls were used:

TABLE 3
Test Molecules and Controls

| Test | |
|---|---|
| Molecule | Description |
| TCE1 | See Table 2 |
| TCE2 | See Table 2 |
| TCE3 | See Table 2 |
| TCE4 | See Table 2 |
| TCE5 | See Table 2 |
| TCE6 | See Table 2 |
| TCE7 | See Table 2 |
| Control A | Negative CD3 control: effector null anti-tetanus toxin |
| | mock (irrelevant) arm × CD3 arm Clone A of the |
| | invention, hIgG1 with LALADS, KiH, cLC |
| Control B | Negative CD3 control: effector null anti-tetanus toxin |
| | mock (irrelevant) arm × CD3 arm Clone B of the |
| | invention, hIgG1 with LALADS, KiH, cLC |
| Control C | Negative CD3 control: effector null anti-tetanus toxin |
| | mock (irrelevant) arm × CD3 arm Clone C of the |
| | invention, hIgG1 with LALADS, KiH, cLC |
| R3Mab × | Positive control TCE: R3Mab is an anti-FGFR3 |
| 38E4v1 | antibody that binds both wildtype and various mutant |
| | forms of FGFR3 (see Qing, J. et al., J Clin Invest. |
| | 2009 May 1; 119(5): 1216-1229) and 38E4v1 is an |
| | anti-CD3 humanized antibody |
| Control D | Positive control TCE: anti-FGFR3 S249C arm (see clone |
| | FGA0002 in US2022/0267452) and 38E4v1 is an anti- |
| | CD3 humanized antibody |
| Control E | Positive control muromonab-OKT3 murine anti-CD3 |
| | Ab (see Norman, D., Therap Drug Monitor. 17(6): |
| | p. 615, December 1995) |
| TT × | Negative control: tetanus toxin antibody (irrelevant) × |
| 38E4v1 | and 38E4v1 is an anti-CD3 humanized antibody |
| HEL10 × | Negative control TCE: HEL10 is hen egg lysosome and |
| 38E4v1 | 38E4v1 is an anti-CD3 humanized antibody |
| FGFR1 | Positive control anti-mouse FGFR1 antibody (Abnova |
| | cat#h00002260-m04) |
| FGFR2 | Positive control anti-mouse FGFR2 isoform IIIb antibody |
| | (R&D Systems cat#MAB665) |
| FGFR4 | Positive control anti-mouse FGFR4 antibody (R&D |
| | Systems cat#MAB685) |

### Example 2: In Vitro Characterization of TCE Binding to Human CD3 and T Cells

This study was performed to test the binding activity of TCEs 1, 2, 4, 5 and 7 on human CD3 expressing Jurkat/NFAT cells and isolated human T cells.

Jurkat NFAT cells were harvested and transferred into 15 mL centrifuge tubes and were counted using cellometer. 30×10⁶ cells were washed once at 300×g for 5 min and were resuspended to 4×10⁶ cells/mL of staining buffer+human Fc block diluted 1:100. Cells were plated at 25 μL per well (100,000 cells/well) into 96-well v bottom plates and incubated on ice for 30 mins.

Antibody dilutions were prepared manually in stain buffer, 10-point, 3-fold dilution series, to 2× final concentration and transferred to cells. After 1 hour incubation on ice, the cells were washed 3× and resuspended with 50 μL/well of AF647 secondary anti-human antibody at 1:200 dilution in stain buffer and incubated on ice protected from light for 45 mins. Cells were washed and incubated with 100 μL/well zombie live/dead violet diluted at 1:400 in 1×PBS for 15 mins on ice. Cells were washed and resuspended with 80 μL/well of stain buffer and 40 μL was collected on the Attune flow cytometer.

PBMCs from two human donors were separately thawed in 37° C. water bath and transferred into 15 mL centrifuge tube containing 7 mL of complete media (RPMI+10% HI FBS+1× Glutamax™) and washed once at 300× g for 5 min. Cells were resuspended in 5 mL RoboSep™ buffer and counted using the Cellometer™ cell viability counter. Cells were transferred to 14 mL polystyrene tubes, washed, and resuspended in 1 mL RoboSep™ buffer for T cell isolation.

T cell isolation was manually performed using human T cell isolation kit by Stemcell™ and EasySep™ magnet. Volume of reagents or buffer used varies and the recommended volume was used as indicated by the kit manual. The final T cells were washed and resuspended in stain buffer and counted for flow staining.

T cells were plated to 150,000 cells/well in the presence of human Fc block and incubated on ice for 30 mins. TCE dilutions were prepared manually in stain buffer, 3-point, 3-fold then 5-fold dilutions, to 2× final concentration and transferred to cells. After 1 hour incubation on ice, the cells were washed 3× and resuspended with PE mouse anti-human IgG secondary antibody at 1:25 dilution in stain buffer and incubated on ice protected from light for 45 mins. Cells were washed and incubated with zombie live/dead violet for 15 min. Cells were washed and resuspended with 80 μL of stain buffer and 50 μL was collected on the Attune™ flow cytometer. TCEs were tested in duplicate within each individual experiment.

Data was analyzed using FlowJo version 10.10.0. The following gating strategy was used: cells→single cells→live cells→raw MFI Geo mean of AF647 or PE was taken and the subtracted average MFI Geo mean of PE secondary only was plotted on GraphPad Prism version 10.1.2.

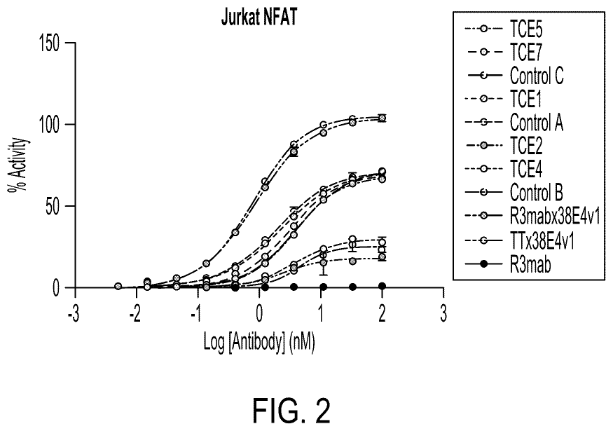

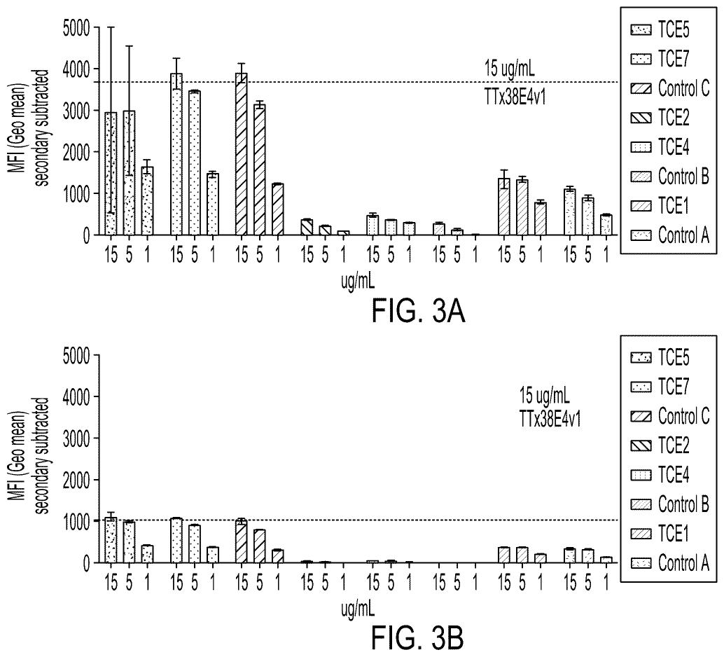

Summary: All TCEs tested showed specific cell binding to both Jurkat NFAT (FIG. 2) and human T cells from two different human donors (FIGS. 3A and 3B). These results confirm that the CD3 arms of the tested TCEs of the invention specifically bind to human CD3.

### Example 3: In Vitro Characterization of TCE Binding to FGFR3 S249C Versus WT FGFR3 and FGFR3 S248C

This study was performed to test the binding activity of TCEs 1, 2, 3, 4, 5, and 7 on different engineered Hela cell lines: Hela-FGFR3 isoform IIIb S249C, Hela-FGFR3 isoform IIIb WT, Hela-FGFR3 isoform IIIc S249C, and Hela-FGFR3 isoform IIIc WT cell lines. In addition, TCEs 1, 2, 4, 5, 6 and 7 in a DEKK backbone were assessed on a Hela-FGFR3 isoform IIIb R248C cell line.

Cells were cultured in complete media (DMEM+10% HI FBS+1× Glutamax™+0.5 μg/mL puromycin) to confluency then harvested from plates with Corning™ CellStripper Dissociation Reagent. Cells were washed with complete media, counted, and incubated with human Fc block (1:100) and Zombie Green™ Fixable Viability Dye (1:1000) in PBS on ice for 20 min, protected from light. Stocks of primary antibody dilutions were prepared by serial dilution in BD Staining Buffer, starting at 75 nM, 1:4 dilution, 8 points. Cells were aliquoted into 96-well plates @ 100,000 cells per well, 3 plates for each cell line. Plates were spun at 1800 RPM×3 min and supernatant decanted. Cells were resuspended in 100 μL primary antibody and incubated 1 hr on ice, protected from light. Cells were washed 2× then resuspended in 100 μL secondary Ab (anti-hu-AF647, 1:500) and incubated 45 min on ice, protected from light. Cells were washed 3×, then resuspended in 100 μL Cytofix™ and incubated on ice 20 min, protected from light. Plates were centrifuged briefly (1800 RPM×3 min) to pellet cells, then plates were flicked to remove Cytofix™. Cells were resuspended in 150 μL BD staining buffer and plates were stored at 4° C., protected from light, until sample acquisition on Attune cytometer. Data were analyzed in FlowJo™ and values were plotted in Graph Pad Prism. All TCEs were tested in duplicate within each individual experiment.

Data were analyzed using FlowJo version 10.10.0. The following gating strategy was used: cells (FSC-A vs SSC-A)→single cells (SSC-A vs SSC-H)→live cells (SSC-H vs Zombie Green LD-H)→Σ Median of AF647-H. The average Σ Median of AF647-H secondary only wells (background, 4 wells) was calculated for each plate and subtracted from Σ Median of AF647-H for each well. The average Σ Median of AF647-H for 75 nM R3Mabx38E4v1 wells (max controls, 4 wells) was then calculated for each plate. The normalized % activity for each sample was calculated and normalized to the average Σ Median of AF647-H for 75 nM R3Mabx38E4v1 max control wells for each plate. Values for % activity were then plotted in GraphPad Prism version 10.1.2.

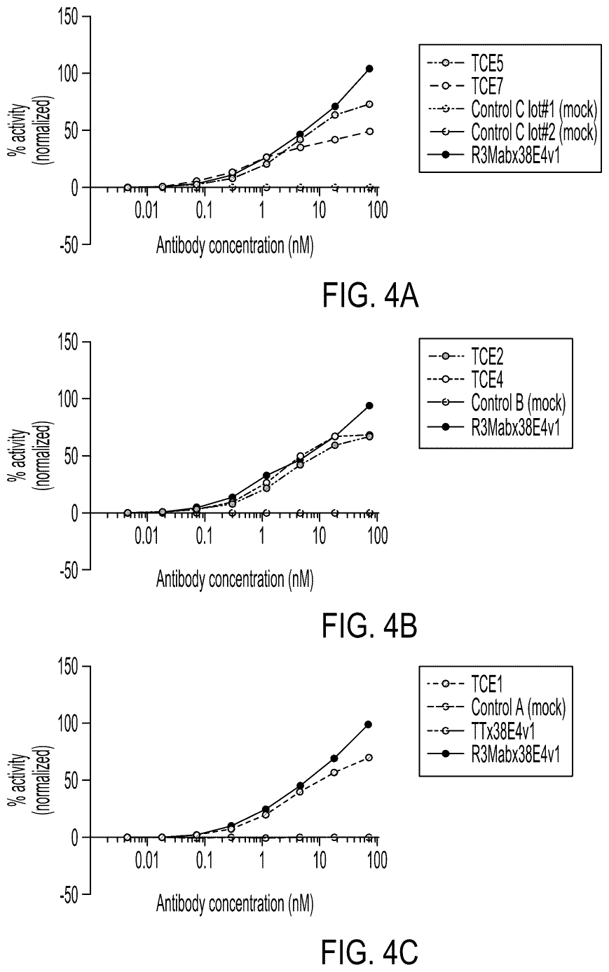

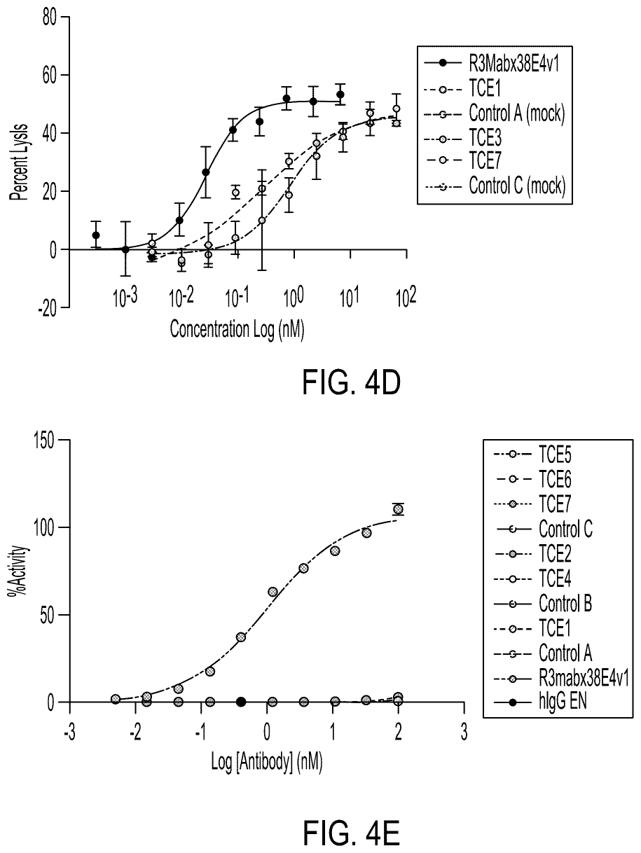

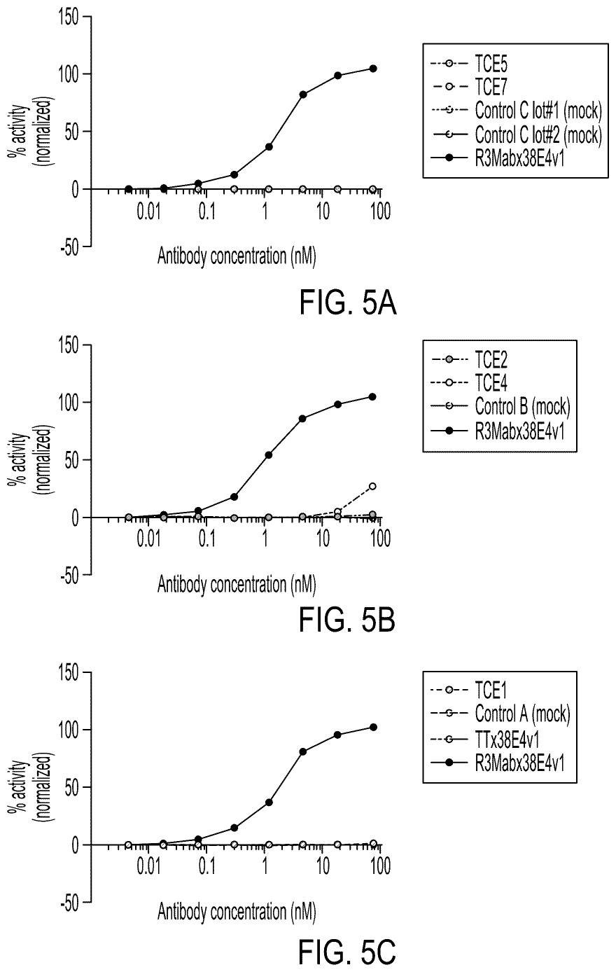

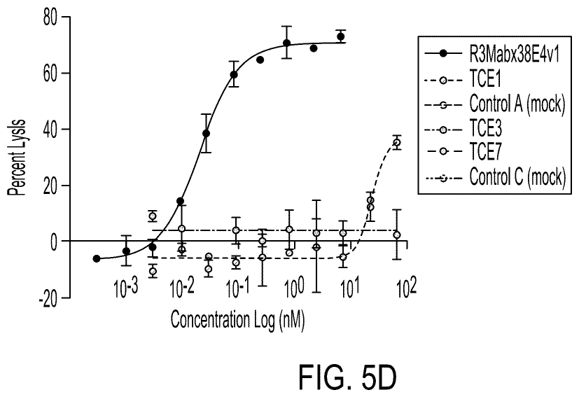

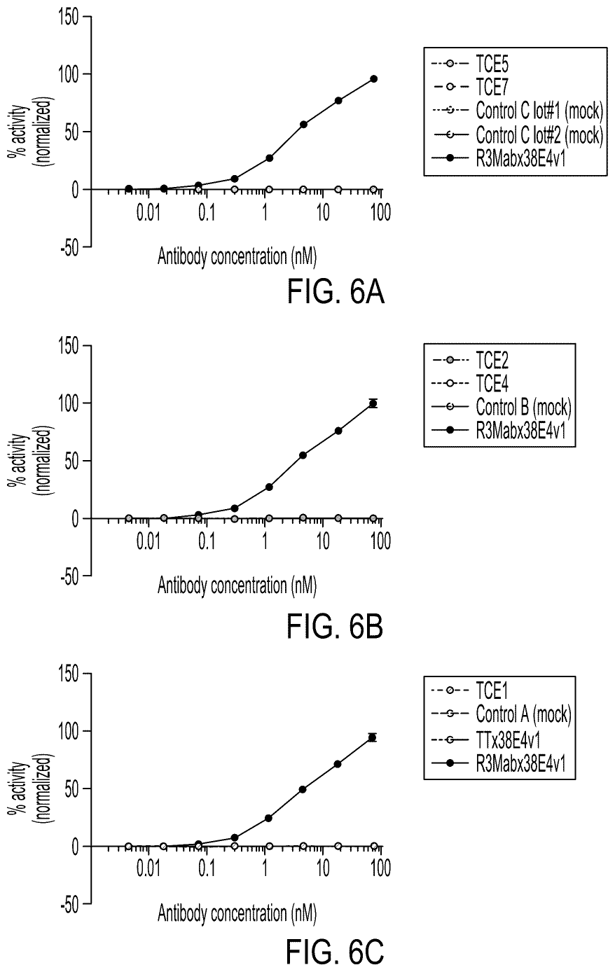

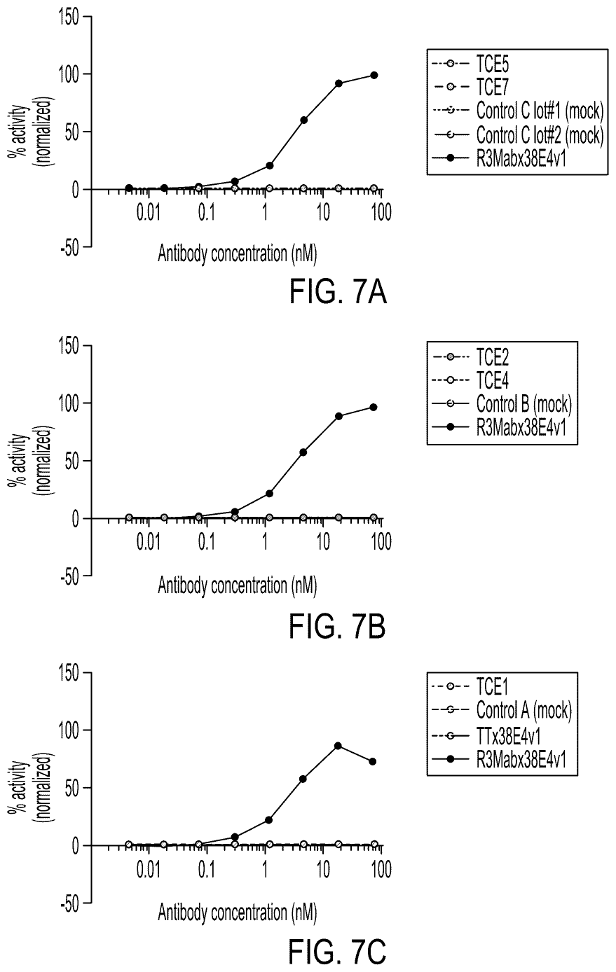

Summary: All TCEs tested showed specific binding to Hela-FGFR3IIIb S249C cells (FIG. 4A-D), with no specific binding observed on Hela-FGFR3IIIb WT cells (FIG. 5A-D), Hela-FGFR3IIIc S249C (FIG. 6A-C), or Hela-FGFR3IIIc WT cells (FIG. 7A-C). Further, TCEs with the DEKK backbone did not demonstrate specific binding to FGFR3IIIb with the S248C mutation (FIG. 4E). Together these results confirm that the TCEs tested specifically bind to Hela-FGFR3 isoform IIIb S249C cells.

### Example 4: In Vitro Characterization of Cell Binding Activity on BdEC Cells

This study was performed to test the binding of TCEs 1, 2, 4, 5 and 7 to FGFR3 WT expressing normal bladder primary epithelial BdEC cells which do not express FGFR3 S249C.

The BdEC cell line was obtained from Oncocell™ and were cultured in Bladder Epithelial Basal Medium (ATCC) supplemented with Bladder Epithelial Growth Kit (ATCC), Glutamax™, and Antibiotic-Antimycotic to confluency and harvested from plates with TrypLE™ Express Enzyme. Cells were washed with complete media, counted, and incubated with human Fc block (1:100) and Zombie Green™ Fixable Viability Dye (1:1000) in PBS on ice for 20 min, protected from light. Stocks of primary antibody dilutions were prepared by serial dilution in BD Staining Buffer, starting at 75 nM, 1:4 dilution, 8 points. Cells were aliquoted into 96-well plates @ 100,000 cells per well, 3 plates total. Plates were spun at 1800 RPM×3 min and supernatant decanted. Cells were resuspended in 100 μL primary antibody and incubated 1 hr on ice, protected from light. Cells were washed 2× then resuspended in 100 μL secondary Ab (anti-hu-AF647, 1:500) and incubated 45 min on ice, protected from light. Cells were washed 3×, then resuspended in 100 μL Cytofix™ and incubated on ice 20 min, protected from light. Plates were centrifuged briefly (1800 RPM×3 min) to pellet cells, then plates were flicked to remove Cytofix “. Cells were resuspended in 150 μL BD staining buffer and plates were stored at 4° C., protected from light, until sample acquisition on Attune” cytometer. Data were analyzed in FlowJo™ and values were plotted in Graph Pad™ Prism. TCEs were tested in duplicate within each individual experiment.

The following gating strategy was used: cells (FSC-A vs SSC-A) à single cells (SSC-A vs SSC-H) à live cells (SSC-H vs Zombie Green LD-H) à Σ Median of AF647-H. The average Σ Median of AF647-H secondary only wells (background, 4 wells) was calculated for each plate and subtracted from Σ Median of AF647-H for each well. The average Σ Median of AF647-H for 75 nM R3Mabx38E4v1 wells (max controls, 4 wells) was then calculated for each plate. The normalized % activity for each sample was calculated and normalized to average Σ Median of AF647-H for 75 nM R3Mabx38E4v1 max control wells for each plate.

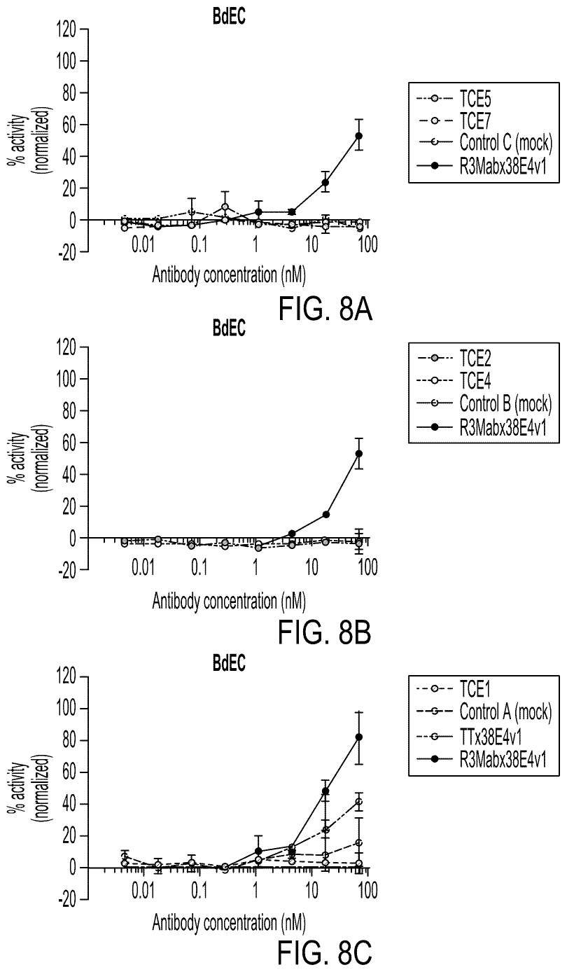

Summary: None of the TCEs tested specifically bound to normal bladder epithelial BdEC cells (FIGS. 8A-C), whereas the positive control R3Mabx38E4v1 (FIGS. 8A-C) demonstrated specific binding (FIG. 8C). Therefore, the TCEs of the invention do not demonstrate specific binding to normal bladder cells.

### Example 5: In Vitro Characterization of Binding to RT112 Cells

This study was performed to test the binding activity of TCEs 1, 2, 4, 5 and 7 on RT112 bladder cancer cells. RT112 cells endogenously express a FGFR3IIIb-TACC3 fusion protein, where the extracellular domain is similar to FGFR3 WT.

RT112 cell line was obtained from DSMZ. These cells were cultured in complete media (RPMI+10% HI FBS+Sodium Pyruvate+1× Glutamax™) to confluency then harvested from plates with Corning™ CellStripper™ Dissociation Reagent. Cells were washed with complete media, counted, and incubated with human Fc block (1:100) and Zombie Green™ Fixable Viability Dye (1:1000) in PBS on ice for 20 min, protected from light. Stocks of primary antibody dilutions were prepared by serial dilution in BD Staining Buffer, starting at 75 nM, 1:4 dilution, 8 points. Cells were aliquoted into 96-well plates @ 100,000 cells per well, 3 plates total. Plates were spun at 1800 RPM×3 min and supernatant decanted. Cells were resuspended in 100 μL primary antibody and incubated Ihr on ice, protected from light. Cells were washed 2× then resuspended in 100 μL secondary Ab (anti-hu-AF647, 1:500) and incubated 45 min on ice, protected from light. Cells were washed 3×, then resuspended in 100 μL Cytofix™ and incubated on ice 20 min, protected from light. Plates were centrifuged briefly (1800 RPM×3 min) to pellet cells, then plates were flicked to remove Cytofix™. Cells were resuspended in 150 μL BD staining buffer and plates were stored at 4° C., protected from light, until sample acquisition on Attune cytometer. TCEs were tested in duplicate within each individual experiment.

Data were analyzed in FlowJo™ and values were plotted in Graph Pad™ Prism. TCEs were tested in duplicate within each individual experiment. Data were analyzed using FlowJo version 10.10.0. The following gating strategy was used: cells (FSC-A vs SSC-A) à single cells (SSC-A vs SSC-H) à live cells (SSC-H vs Zombie Green LD-H) a Σ Median of AF647-H. The average Σ Median of AF647-H secondary only wells (background, 4 wells) was calculated for each plate and subtracted from Σ Median of AF647-H for each well. The average Σ Median of AF647-H for 75 nM R3Mabx38E4v1 wells (max controls, 4 wells) was then calculated for each plate. The normalized % activity for each sample was calculated and normalized to average Σ Median of AF647-H for 75 nM R3Mabx38E4v1 max control wells for each plate.

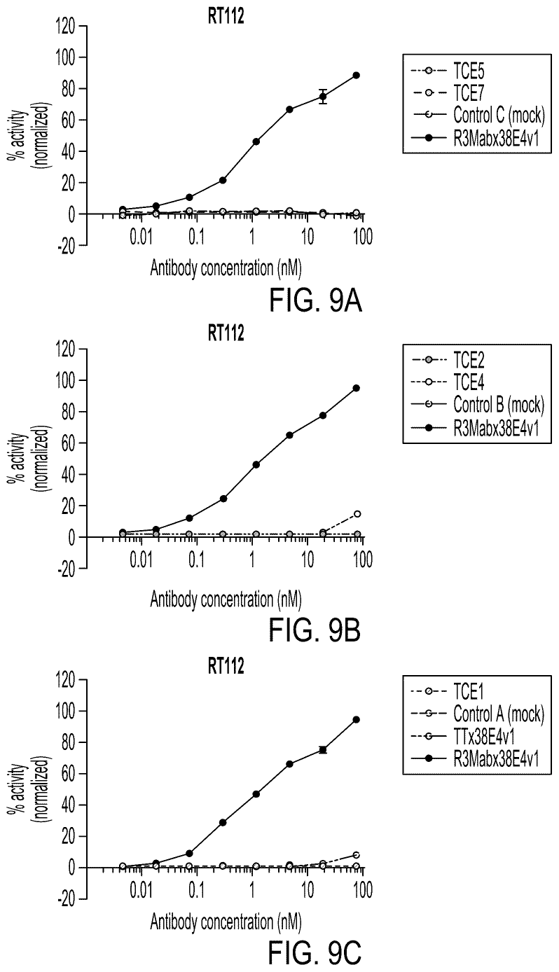

Summary: None of the TCEs tested specifically bound to the FGFR3-TACC3 fusion expressed on the RT112 cells (FIGS. 9A-C), whereas the positive control R3Mabx38E4v1 demonstrated robust specific binding (FIGS. 9A-C).

### Example 6: In Vitro Characterization of Binding to FGFR1, FGFR2 and FGFR4

This study was performed to test the binding specificity of TCEs 1, 2, 4, 5, 6 and 7 on DEKK backbones against HEK293T cells engineered to express human: (a) FGFR1 isoform IIIb, (b) FGFR1 isoform IIIc, (c) FGFR2 isoform IIIb, (d) FGFR2 isoform IIIc, (e) FGFR3 isoform IIIb wild-type, (f) FGFR3 isoform IIIb S249C, (g) FGFR3 isoform IIIc wild-type, (h) FGFR3 isoform IIIc S249C, and (i) FGFR4, all by flow cytometry.

HEK293-FGFR2IIIc cells were treated with 1 ug/ml doxycycline hyclate (Tocris Bioscience, catalog #4090, Lot #4A/250772) overnight before harvesting. On the day of the assay, all cells were dissociated using 3 mL of cell dissociation buffer and transferred into 15 mL centrifuge tubes containing 7 mL of complete media (DMEM+10% HI FBS+1× Glutamax). Cells were counted using a cellometer and 15-30×10⁶ cells were washed once at 300× g for 5 min. Cells were resuspended to 4×10⁶ cells/mL of staining buffer+human Fc block diluted 1:100. Cells were plated at 25 μL per well (100,000 cells/well) into 96-well v bottom plates and incubated on ice for 30 mins.

Antibody dilutions were prepared manually in stain buffer, 10-point, 3-fold dilution series, to 2× final concentration and transferred to cells. After 1 hour incubation on ice, the cells were washed 3× and resuspended with 50 μL/well of AF647 secondary anti-human antibody at 1:200 dilution in stain buffer or AF647 secondary anti-mouse antibody at 1:500 (only for FGFR1 Abnova and FGFR4 MAB685) in stain buffer and incubated on ice protected from light for 45 mins. Cells were washed and incubated with 100 μL/well zombie live/dead violet diluted at 1:400 in 1×PBS for 10-15 mins on ice. Cells were washed and resuspended with 80 μL/well of stain buffer and 40 μL was collected on the Attune flow cytometer. TCEs were tested in duplicate within each individual experiment.

Data was analyzed using FlowJo version 10.10.0. The following gating strategy was used: cells→single cells→live cells→raw MFI Geo mean of AF647 was taken and the subtracted average MFI Geo mean of AF647 secondary only was plotted on GraphPad Prism version 10.1.2.

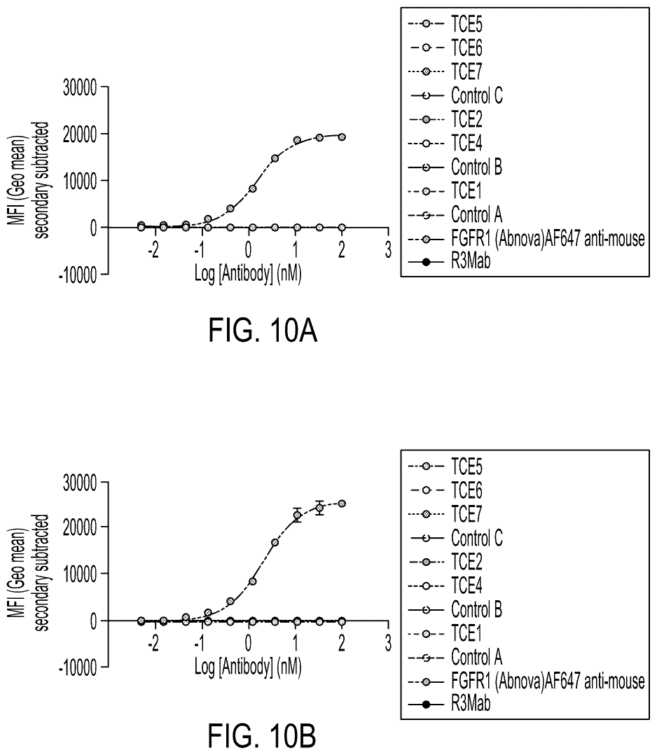

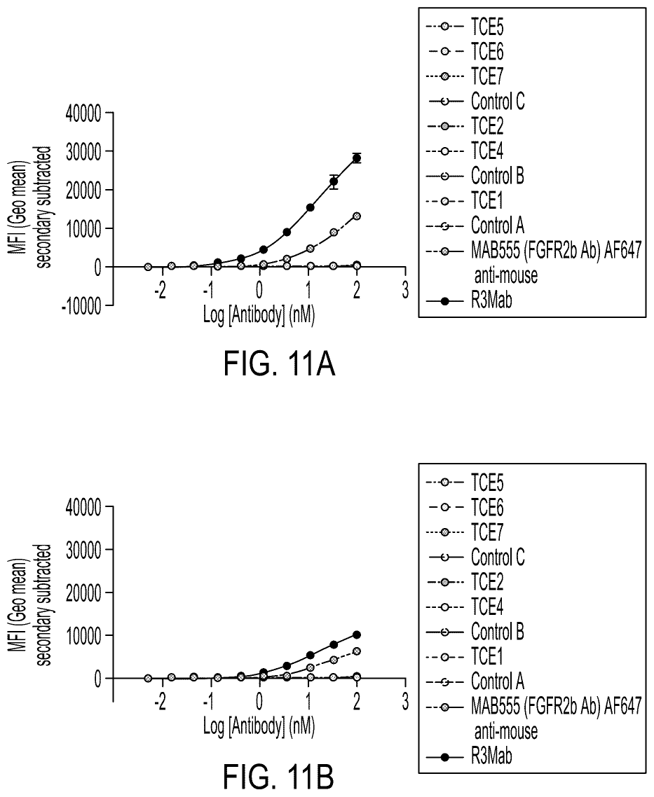

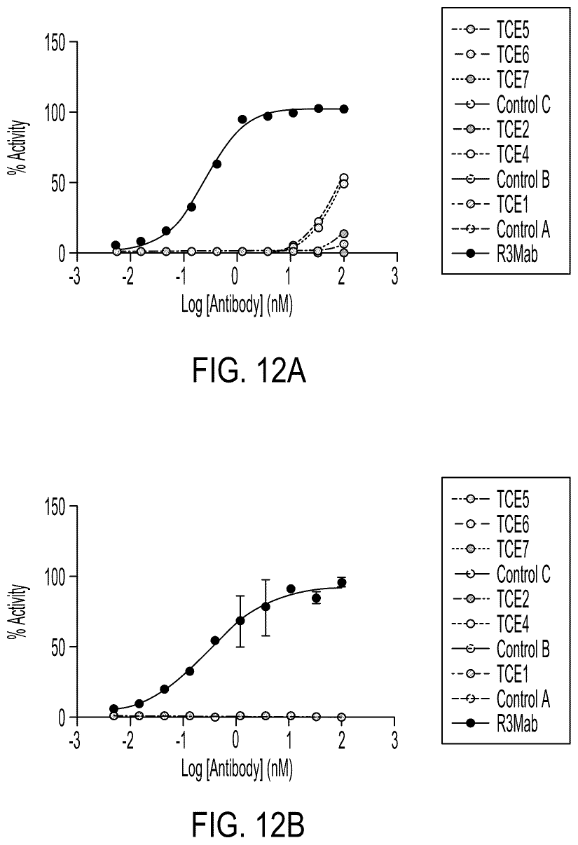

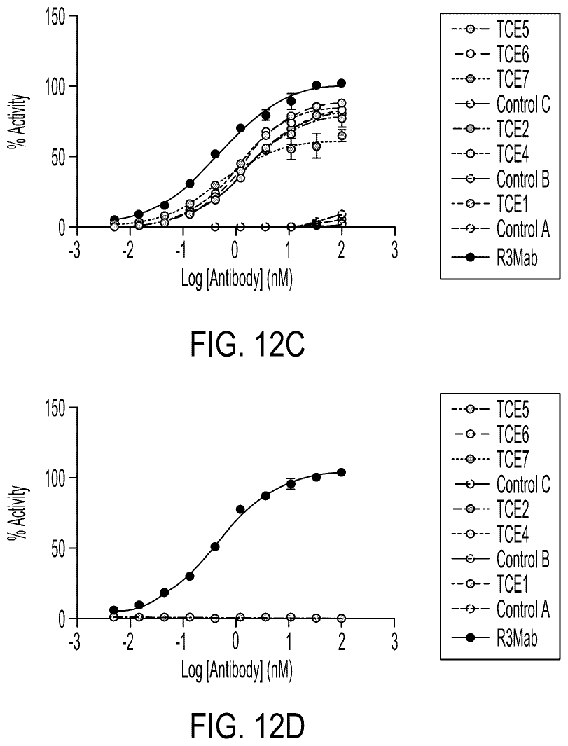

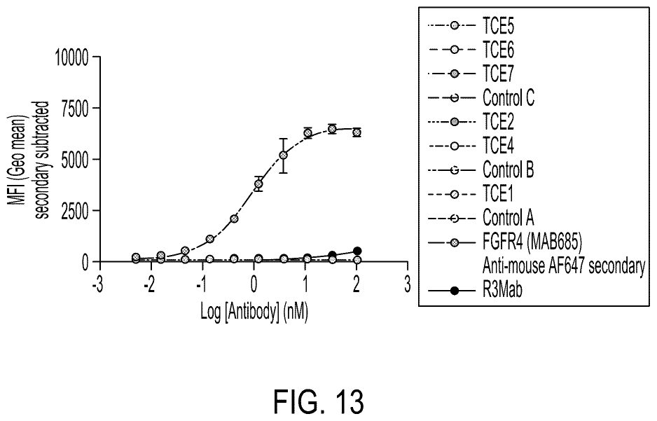

Summary: All the TCEs tested showed specific binding to HEK293T cells expressing FGFR3IIIb S249C (FIG. 12C) but none demonstrated any specific binding to: HEK293T-FGFR1IIIb (FIG. 10A), HEK293T-FGFR1IIIc (FIG. 10B), HEK293T-FGFR2IIIb (FIG. 11A), HEK293T-FGFR2IIIc (FIG. 11B), or HEK293T-FGFR4 cells (FIG. 13) confirming TCE binding specificity to FGFR3IIIb S249C expressing cells. There was also no specific binding observed to FGFR3IIIc WT (FIG. 12B) or FGFR3IIIc S249C cells (FIG. 12D). Low binding was detected with TCEs 4 and 6 on the HEK293T-FGFR3IIIb WT cells but only at the highest tested concentrations (FIG. 12A), suggesting such binding was non-specific.

### Example 7: Evaluation of Human CD3 Protein Binding by ELISA

This study was performed to test the protein binding activity of TCEs 1, 2, 4, 5 and 6 with human CD3 heterodimer epsilon delta recombinant protein and human CD3 heterodimer epsilon gamma recombinant protein by ELISA.

96-well plates were coated with a volume of 100 μl/well of 1 μg/ml of each human CD3 recombinant protein (in PBS). Plates were sealed and incubated overnight in 4° C. on a plate shaker. Next day, plates were washed 3× with 1×PBS-Tween (contains 0.05% Tween). Plates were then blocked with 300 μl/well of blocking buffer (3% BSA in PBS) for 2 hours at room temperature with orbital shaking. TCE dilutions in assay buffer (1% BSA in PBS) were prepared manually, 10-point 3-fold dilution with a starting concentration of 15 μg/ml or100 nM. TCEs used for plate normalizations were also prepared at 15 μg/ml. Blocking buffer was decanted and antibody dilutions (including plate controls or assay buffer wells) were added to the plates at 100 μl/well and incubated for 1 hour at room temperature with orbital shaking. Peroxidase-conjugated anti-human IgG F(ab′) 2 secondary was prepared in assay buffer at 1:10,000 dilution. Plates were washed 3× (using the same wash method as above). Anti-human secondary was added to the plates at 100 μl/well and incubated for 1 hour at room temperature with orbital shaking. Plates were then washed 3× (using the same wash method as above). TMB substrate was added to the plates at 100 ul/well for 5 minutes. Then 100 μl/well of stop solution was added and plates were read at 450 nm on the SpectraMax™ M5e plate reader using the SoftMax Pro™ program. All TCEs were tested in duplicate in one experiment.

Raw values from the plate reader were analyzed as follows. Background (value of assay buffer-only wells) was subtracted from all values. Normalization across all plates were performed using plate control wells, in which “max activity” wells included 15 μg/ml R3Mabx38E4v1 and “min activity” wells included 15 μg/ml of a negative control (human IgG1 EN). Percent activity was calculated as %=((sample value−min)/(max−min))*100. Titrated antibody concentrations (nM) were transformed to logarithm of concentrations. Curves were fitted by the non-linear regression log (agonist) vs. response-variable slope (four parameters) model in Graphpad™ Prism.

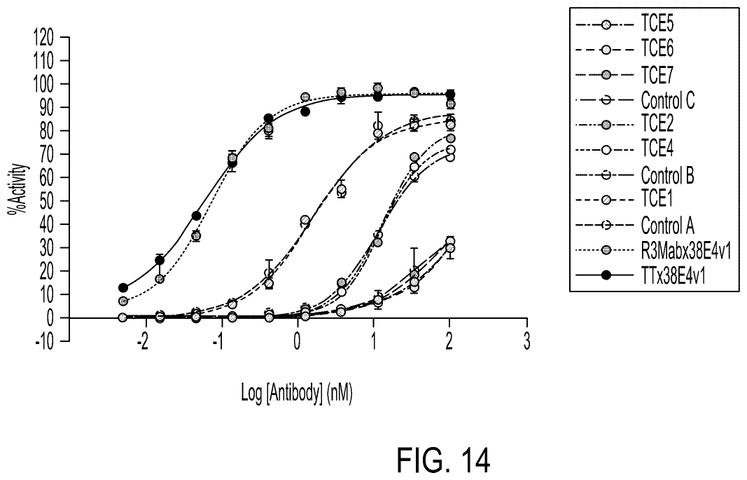

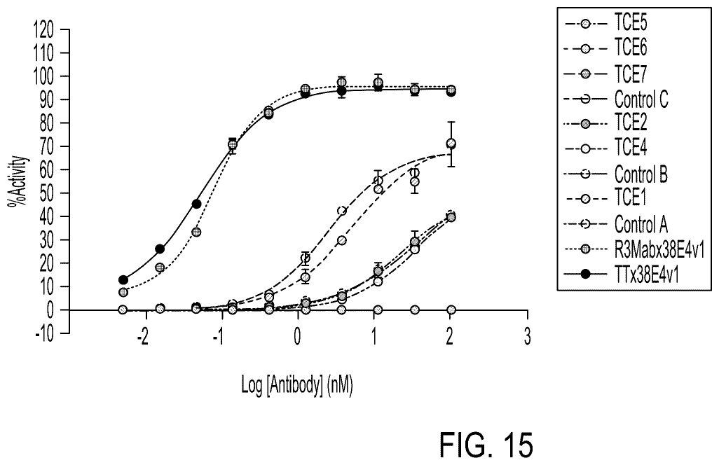

Summary: All TCEs tested specifically bound to human CD3 heterodimer epsilon delta by ELISA (FIG. 14). TCEs with CD3 arms comprising Clones A and B (TCEs 1, 2 and 4) showed specific binding to human CD3 heterodimer epsilon gamma recombinant protein (FIG. 15), whereas TCEs with the Clone C CD3 arm did not (TCE5 and TCE6 as shown in FIG. 15).

### Example 8: In Vitro Characterization of Protein Binding by ELISA

This study was performed to test the protein binding activity of TCEs 1, 2, 4 and 5 to human FGFR3IIIb S249C recombinant protein, human FGFR3IIIc S249C recombinant protein, human FGFR3 IIIb WT recombinant protein, and human FGFR3IIIc WT recombinant protein by ELISA.

Pierce Streptavidin coated clear 96-well plates (pre-blocked) were washed three times with 1×PBS-Tween (contains 0.05% Tween) using a plate washer. Biotinylated recombinant protein solutions were generated at 1 ug/mL in 1×PBS and 100 μL per well of each solution was added to capture proteins onto streptavidin plates. Plates were then sealed and incubated for 1.5 hours at room temperature with orbital shaking. TCEs were serially titrated in assay buffer (1% BSA in PBS); an 11-point 3-fold dilution series starting at 15 μg/mL (or 100 nM) was generated for each antibody. Plates were decanted/washed three times as above and antibody dilutions (including assay buffer-only wells) were added at 100 μL per well. Plates were sealed and incubated for 1.5 hours at room temperature with orbital shaking. Peroxidase-conjugated anti-human IgG F(ab′) 2 secondary was prepared in assay buffer at 1:5,000 dilution. Plates were decanted/washed three times as above and anti-human secondary was added at 100 μL per well. Plates were sealed and incubated for 1 hour at room temperature with orbital shaking. Finally, plates were decanted/washed three times as above and TMB substrate was added at 100 μL per well for approximately 5 minutes. The assay was terminated with the addition of 100 μL per well stop solution and absorbance was measured at 450 nm on the SpectraMax™ M5e plate reader using the SoftMax Pro™ program. All TCEs were tested in duplicate within each individual experiment.

Raw values from the plate reader were analyzed as follows. Background (value of assay buffer-only wells) was subtracted from all values. Graphs were generated in GraphPad™ Prism v.10. Titrated antibody concentrations (nM) were transformed to logarithm of concentrations. Curves were fitted by the non-linear regression log (agonist) vs. response-variable slope (four parameters) model.

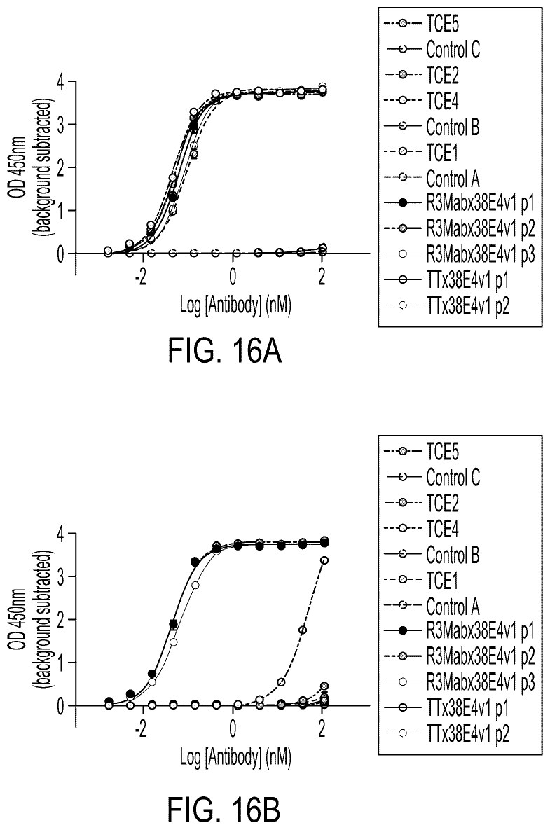

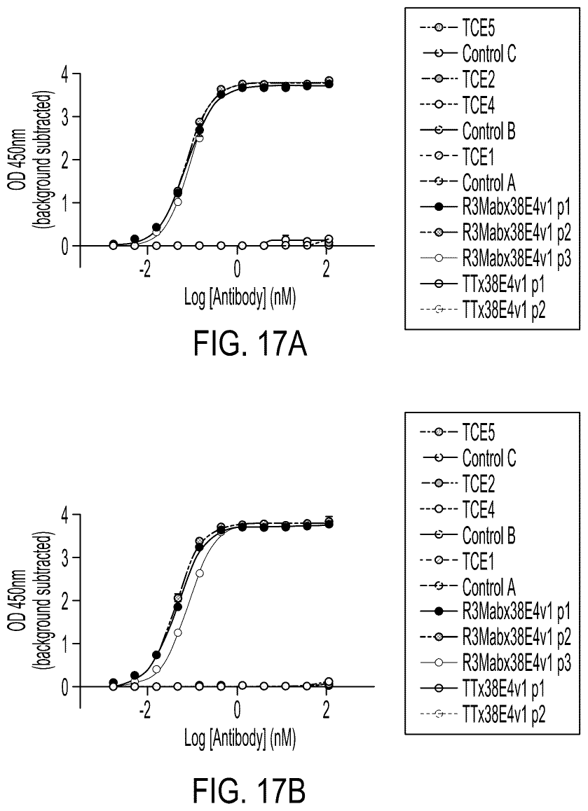

Summary: All TCEs tested specifically bound to human FGFR3IIIb S249C protein (FIG. 16A). TCEs 1, 4 and 5 showed no specific binding to human FGFR3IIIb WT protein (FIG. 16B). TCE2 displayed binding to human FGFR3IIIb WT recombinant protein but only at the highest antibody concentrations used, suggesting this binding was non-specific (FIG. 16B). None of the TCEs tested bound human FGFR3IIIc S249C (FIG. 17A) or FGFR3IIIc WT recombinant protein (FIG. 17B).

### Example 9: Ex Vivo Efficacy Study in Bladder Cancer Organoid Models

This study was performed to evaluate the ex vivo cytotoxic activity of TCEs 1, 2, 3, 4, 5 and 7 in patient-derived FGFR3 S249C+SCBO-1 and WT+SCBO15 bladder cancer organoid models co-cultured with two different PBMC donors. The CellTiter-Glo™ luminescent cell viability assay was used to evaluate the cytotoxic effects.

The FGFR3 S249C+SCBO-1 is a non-invasive tumor type and FGFR3 WT SCBO-15 is an invasive tumor type bladder patient-derived organoid models, both licensed from Columbia University. Organoids were grown in “full” media (+EGF) with 5% Matrigel in ultra-low attachment plates. However, EGF was removed from the media for the Organoid/PBMC co-culture assay.

Organoid/PBMC cytotoxicity assay: SCBO organoids were dissociated into single cells using Tryple™, neutralized with D-10, centrifuged, and resuspended in organoid media without EGF. Single cells were counted, and 2500 cells/well were plated in 100 μl media (without EGF) with 5% Matrigel in cold 96-well tissue culture plates. Plates were incubated at 370 C in 5% CO₂ overnight. The following day, PBMCs were thawed and resuspended in RPMI+10% FBS media with IL2 (50U/ml). 25000 PBMCs in 25 μl RPMI+10% FBS+IL2 (50U/ml) media per well were added (target to effector ratio 1:10). Each TCE was serially diluted 1:4 in RPMI+10% FBS-IL2 with the highest concentration of 30 μg/ml (6×). 25 μl of 6× diluted TCEs were added per well to achieve the final TCE concentration range from 0.005 ug/ml to 5 ug/ml (4-fold dilution, six dilutions. 15 ug/ml=100 nM) in 150 μl total volume per well. The final 150 μl organoid/PBMC co-culture composed of 100 μl of SCBO cells in organoid media without EGF+25 μl PBMCs in RPMI+10% FBS+IL2 (50 U/ml))+25 μl TCE in RPMI+10% FBS-IL2. After seven days of treatment, plates were washed 2× with PBS to remove immune cells, and 60 μl of DMEM media+60 μl of CellTiter-Glo™ 3D reagent was added per well. Plated were incubated at room temperature for 1 hour. For SCBO1, 90 μl of the mix was transferred from 96-well regular tissue culture plates to a 384-well white plate. SCBO15 organoids were co-cultured with PBMCs in 96-well white plates; therefore, luminescence was directly read. Luminescence was measured using a SpectraMax™ M5e reader. In this study, the TCEs were tested in triplicate for each PBMC donor on three plates, with one replicate per plate. However, for the SCBO1 model, one replicate plate per PBMC donor was excluded during the analysis due to interference in luminescence readouts from the live cell imaging dye added to one of the three plates. Dose-response curves were, therefore, plotted based on duplicate readouts per TCE per PBMC donor.

Luminescence was measured by SpectraMax™ M5e with SoftMax Pro 5.4 software. The percentage of cell viability was calculated against no treatment (tumor cells+PBMC-TCE) as 100% viable cells. GraphPad™ Prism was used to analyze the data. EC50 was determined via log (inhibitor) vs. normalized response variable slope curve fitting. Maximum drug response (MaxR) was calculated as the percentage of viable cells at the highest TCE concentration of 5 ug/ml. In the case of the hook effect, MaxR was adjusted to reflect maximum cell death (aka minimum viable cells) in the tested dose range. Normalized AUC (nAUC) was calculated as a ratio of AUC for each TCE dose-response curve vs. the total possible area for the tested dose range.

TABLE 4
Response Data in S249C + SCBO1 Organoid

| | PBMC Donor ID CC01319 | | | PBMC Donor ID CC01344 | | |
|---|---|---|---|---|---|---|
| TCE Tested | EC50 (nM) | MaxR | nAUC | EC50 (nM) | MaxR | nAUC |
| TCE5 | 40.76 | 53.02* | 0.87 | 9.572 | 49.47 | 0.79 |
| TCE2 | 13.49 | 50.13 | 0.87 | 5.971 | 42.82 | 0.82 |
| TCE1 | 4.785 | 43.05 | 0.79 | 2.169 | 37.07 | 0.73 |
| TCE4 | 6.948 | 46.38 | 0.81 | 2.916 | 39.68 | 0.75 |
| Control A | ND | 100 | 1.04 | ND | 99.99 | 1.04 |
| Control B | ND | 100.9 | 1.04 | ND | 99.39 | 1.05 |
| Control C | ND | 101.1 | 1.03 | ND | 103.3 | 1.04 |
| R3Mabx38E4v1 | 0.06352 | 15.11 | 0.43 | 0.04988 | 9.658 | 0.41 |
| TCE7 | 1.486 | 32.65 | 0.70 | 0.9198 | 30.23 | 0.66 |

ND = Not detected.

TABLE 5
Response Data in FGFR3 WT SCBO15 Organoid

| | PBMC Donor | | | PBMC Donor | | |
|---|---|---|---|---|---|---|
| | ID CC01319 | | | ID CC01344 | | |
| | EC50 | | | EC50 | | |
| TCE Tested | (nM) | MaxR | nAUC | (nM) | MaxR | nAUC |
| TCE5 | ND | 100 | 1 | ND | 100 | 1 |
| TCE2 | ND | 100 | 1 | ND | 100 | 1 |
| TCE1 | ND | 100 | 1 | ND | 100 | 1 |
| TCE4 | ND | 100 | 1 | ND | 100 | 1 |
| Control A | ND | 100 | 1 | ND | 100 | 1 |
| Control B | ND | 100 | 1 | ND | 100 | 1 |
| Control C | ND | 100 | 1 | ND | 100 | 1 |
| R3Mabx38E4v1 | 14.27 | 44.44 | 0.8 | ND | 67.2 | 0.9 |
| TCE7 | ND | 100 | 1 | ND | 100 | 1 |

ND = Not detected.

TABLE 6
Response Data in S249C+ SCBO1 Organoid

| | PBMC Donor | | | PBMC Donor | | |
|---|---|---|---|---|---|---|
| | ID CC01319 | | | ID CC01344 | | |
| | EC50 | | | EC50 | | |
| TCE Tested | (nM) | maxR | nAUC | (nM) | maxR | nAUC |
| TCE1 | 5.47 | 36.7 | 0.77 | 4.42 | 37.93 | 0.75 |
| TCE7 | 4.81 | 38.32 | 0.77 | 7.23 | 47.36 | 0.76 |
| TCE3 | 11.52 | 53.50 | 0.79 | 15.13 | 55.02 | 0.79 |
| R3Mabx38E4v1 | 0.23 | 14.7 | 0.53 | 0.51 | 20.88 | 0.59 |

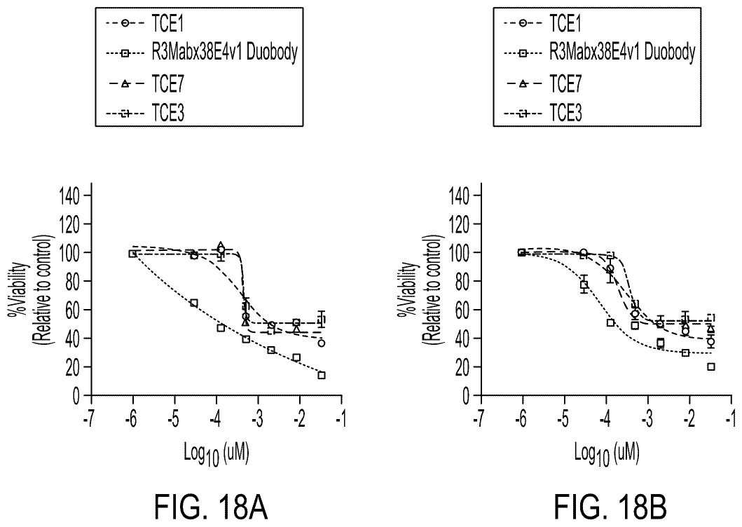

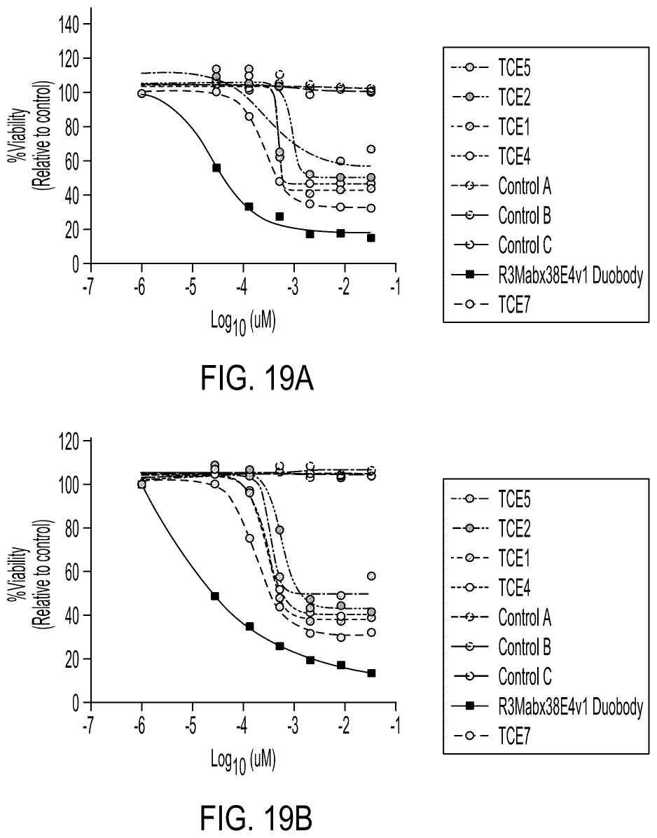

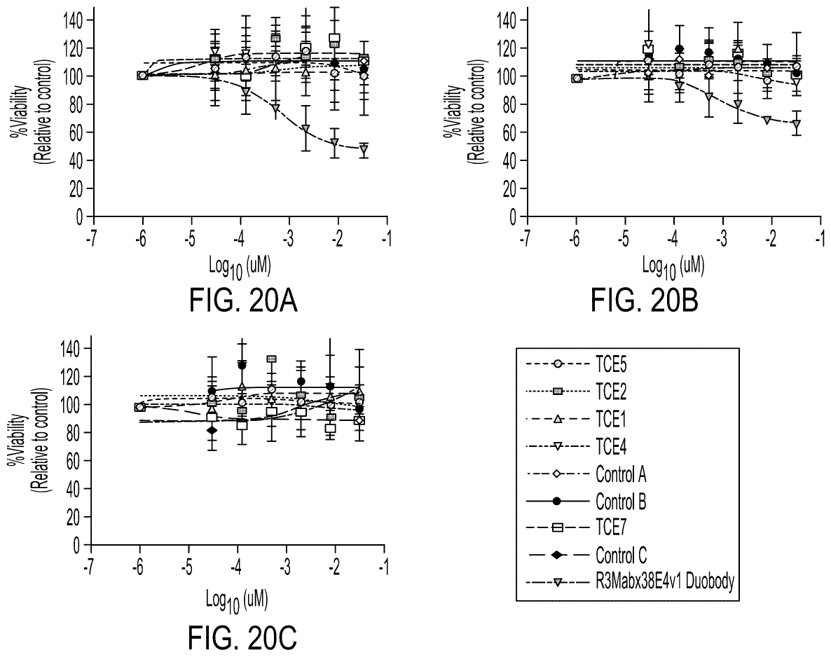

Summary: All TCEs tested demonstrated cytotoxic activity against FGFR3 S249C+ patient-derived bladder organoid model, SCBO1, in an organoid/PBMC co-culture assay (FIGS. 18A and B; FIGS. 19A and B; Tables 4 and 6). The TCEs tested showed no activity in the FGFR3 wild-type SCBO15 organoid model as compared to positive control R3Mabx38E4v1 (FIGS. 20A-B and Table 5). None of the TCEs tested showed activity when no PBMCs were added (FIG. 20C).

### Example 10: In Vitro Cytotoxicity on HeLa Cells

TCEs 1, 2, 4, 5 and 7 were evaluated for cytotoxic activity on HeLa parental cells or HeLa cells engineered to express FGFR3IIIb wildtype or FGFR3IIIb S249C in the presence of human PBMCs.

Human PBMCs were purchased from AllCell®. The HeLa cell line was purchased from ATCC® and engineered HeLa-FGFR3IIIb WT and HeLa-FGFR3IIIb S249C cells were generated using standard cloning techniques.

PBMCs were thawed in RPMI+10% FBS and rested overnight before the experiment. On the day of the experiment, target engineered HeLa cells were harvested with TrypLE and washed with Assay media (RPMI+10% FBS). PBMCs were also washed with assay media. Both PBMCs and target cells were counted and checked for viability with the cell counter ensuring that viability was at least 80%. Cells were then resuspended in media for an E:T ratio of 10:1. Next, 50 μl (5000) target cells were seeded into a 96 well plate followed by adding 25 μl (50,000) effector PBMCs to each well. TCEs were titrated in a separate 96 well U-bottom plate at a start concentration of 10 μg/ml with a 1-to-3 dilution. 25 μl of titrated TCE was added in duplicate to wells containing effector and target cells. Positive control R3Mabx38E4v1 was titrated in the first 2 rows of each plate with a start concentration of 1 μg/ml. Before incubating plates, 2 μl of KILR total lysis control solution was added to designated maximal lysis control wells. Plates were incubated at 37° C. for 48 hours. CellTiter-Glo® Luminescent Cell Viability was assessed by equilibrating plates at room temperature before adding CellTiter-Glo® solution to each well. Luminescence was measured with a luminometer at an integration of 500 ms. Percent lysis values=100−[(Luminescence value of well-Total lysis control)/No TCE control×100]. Data was analyzed using GraphPad Prism version 10.1.2.

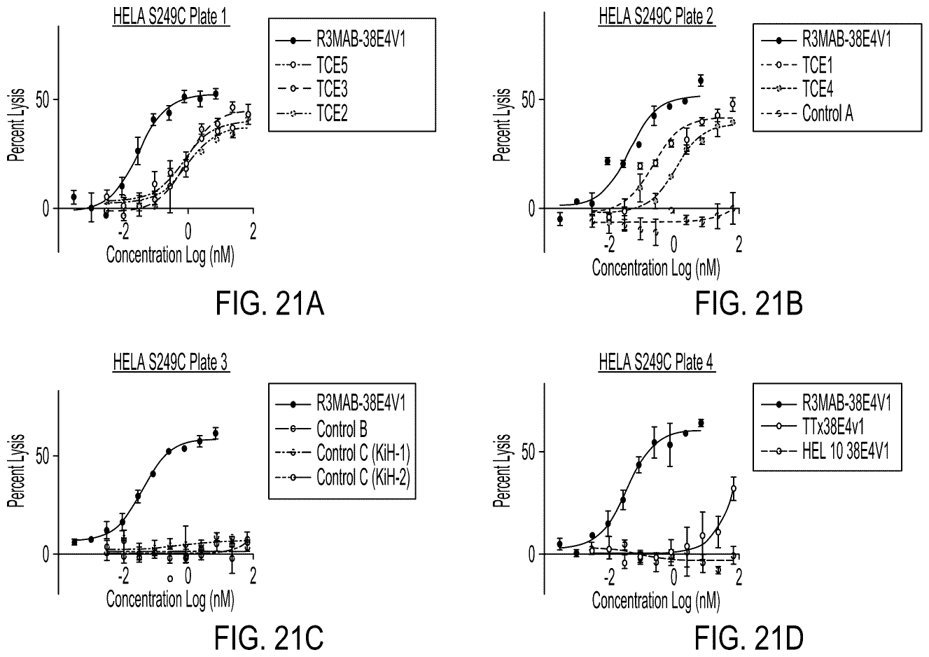

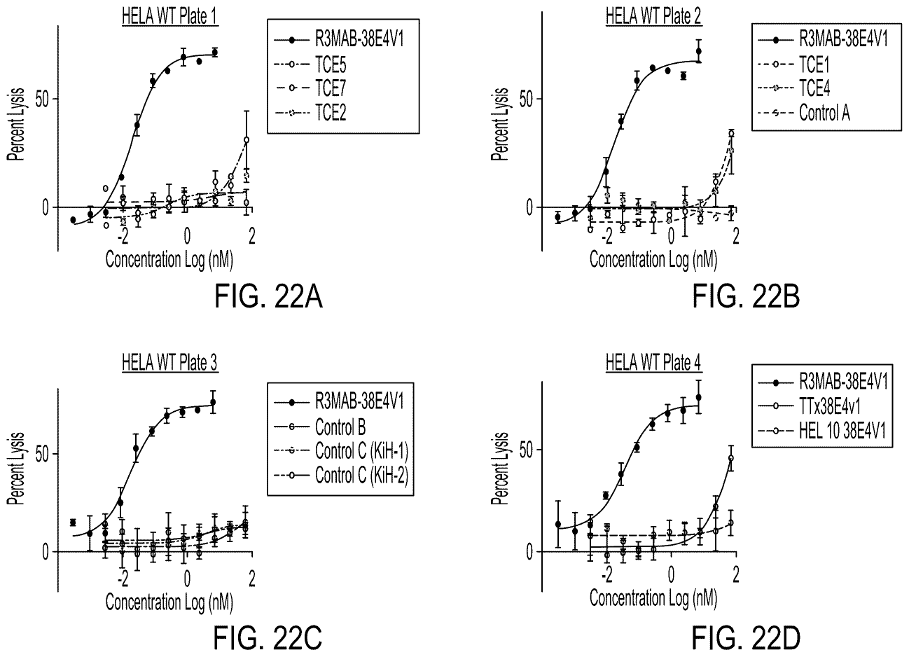

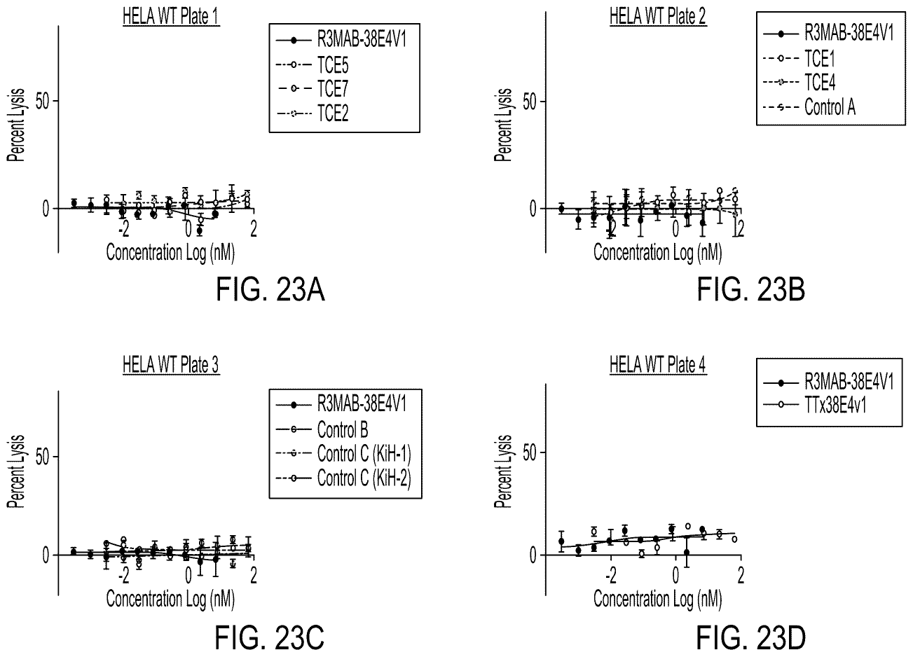

Summary: All TCEs tested demonstrated cytotoxic activity in the presence of human PBMCs against HeLa FGFR3IIIb S249C cells (FIGS. 21A-B) as compared to negative controls (FIGS. 21B, C-D). The TCEs of the invention showed no activity on HeLa-FGFR3IIIb wildtype cells at the low concentrations but showed minimal activity at the highest concentrations tested (FIGS. 22A-B) which is contrasted to both negative and positive controls used (FIGS. 22C-D). No TCE activity was measured to HeLa parental cells alone (FIGS. 23A-B) as compared to both negative and positive controls (FIGS. 23C-D). These results indicate the cytotoxic specificity of the TCEs of the invention to FGFR3IIIb S249C expressing cells.

### Example 11: In Vitro Cytotoxicity on UMUC-14 Cells

TCEs 1, 2, 3, 4, 5 and 7 were assessed for cytotoxic activity in two separate experiments on an endogenously expressing FGFR3IIIb S249C UMUC-14 KILR cells in the presence of human PBMC effector cells.

PBMC donors were purchased from AllCell™. The UMUC-14 KILR line were developed in house using conventional molecular cloning methodologies.

PBMCs were thawed in RPMI+10% FBS and rested overnight before the experiment. On the day of experiment, UMUC-14 target cells were harvested with TrypLE™ and washed with assay media (RPMI+10% FBS). PBMCs were also washed with assay media. Both PBMCs and target cells were counted and checked for viability with the cell counter ensuring that the viability is at least 80%. After cell counting, cells were resuspended in media for an E:T ratio of 10:1. Next, 50 μl (5000) target cells were seeded into a 96 well plate followed by adding 25 μl (50,000) PBMCs to each well. TCEs were titrated in a separate 96 well U-bottom plate at a start concentration of 10 μg/ml with a 1-to-3 dilution in Experiment #1 and 15 μg/ml with a 1-to-3 dilution down to 0.002 μg/ml in Experiment #2. 25 μl of titrated TCE was added in duplicate to wells containing PBMC effector and target cells. R3Mabx38E4v1 was titrated in the first 2 rows of each plate as a positive control with a start concentration of 1 μg/ml. Before incubating plates, 2 μl of KILR total lysis control solution was added to designated maximal lysis control wells. Plates were incubated at 37° C. for 48 hours. To assess cytotoxicity, 100 μl of the KILR assay mix was added to each well. The plates were incubated on a shaker for 1 hour at room temperature away from light before reading luminescence with a luminometer at an integration of 500 ms.

Percent lysis values were calculated as follows: Percent Lysis=(Luminescence value of well-No TCE control)/Total Lysis control×100. Data was analyzed using GraphPad Prism version 10.1.2.

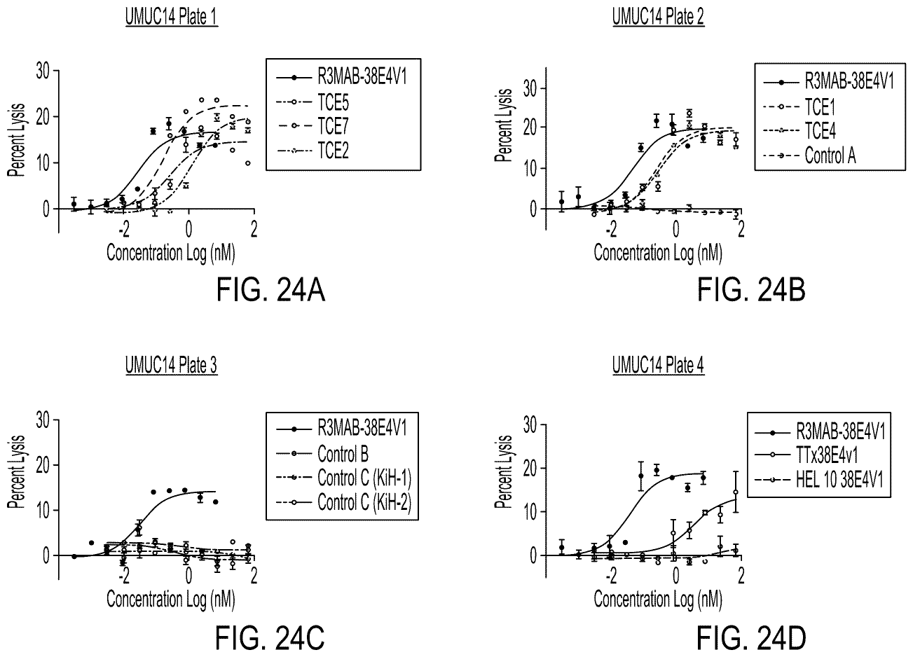

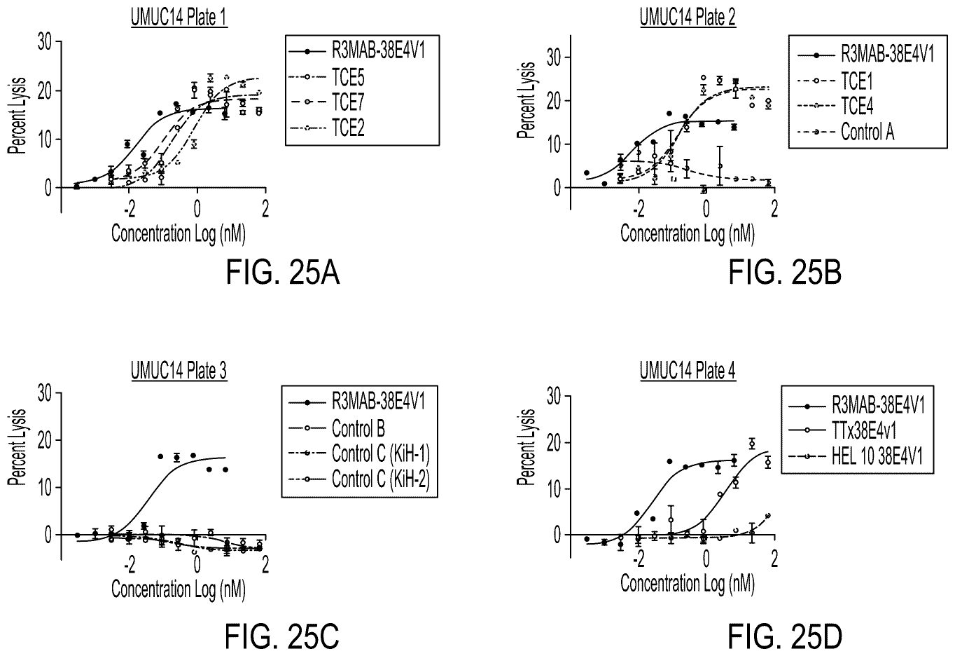

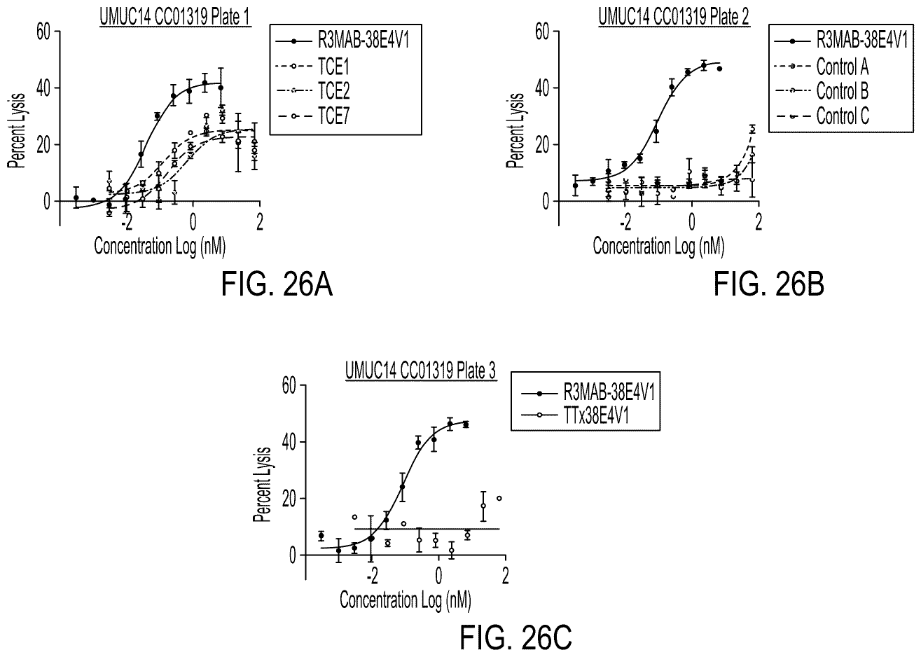

Summary—Experiment #1: In the presence of a 10:1 effector-to-target cell ratio, where PBMC donors were assessed as effector cells, all TCEs tested demonstrated cytotoxicity to the FGFR3IIIb S249C expressing human bladder cancer cell line UMUC-14. All TCEs tested demonstrated comparable cytotoxic activity on UMUC-14 cells that endogenously express FGFR3IIIb S249Cin the presence of all PBMC donors tested (FIGS. 24A-B, 25A-B and 26A). Minimal activity of negative controls used was detected (FIGS. 24B-D, 25B-D and 26B-C). These studies demonstrate the cytotoxicity specificity of the TCEs of the invention towards FGFR3IIIb S249C.

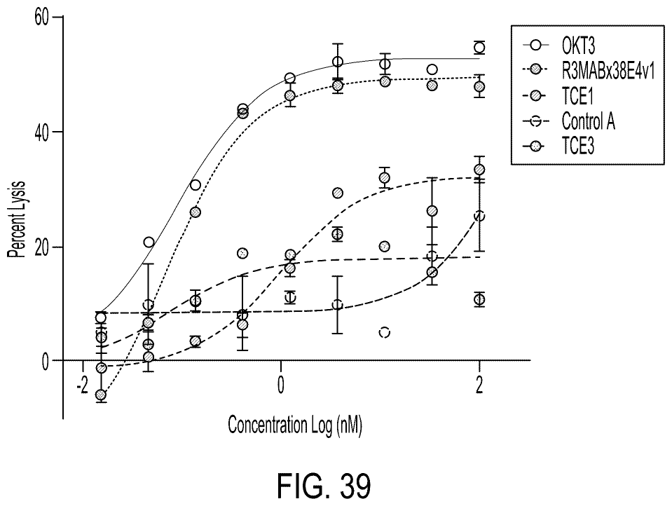

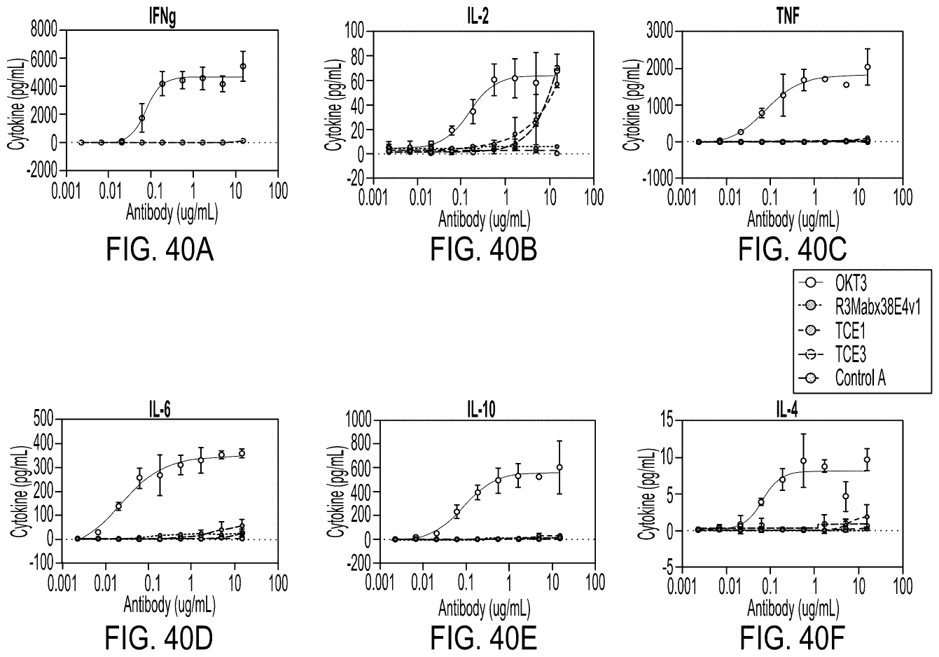

Summary—Experiment #2: TCEs 1 and 3 and controls R3Mabx38E4v1 and Control E all demonstrate cytotoxicity to the FGFR3IIIb S249C expressing human bladder cancer cell line UMUC-14 at all concentrations tested (See FIG. 39). However, TCEs 1 and 3 induced low cytokine production for IFNg, IL-2, TNFa, IL-6, IL-10 and IL-4 in the presence of PBMCs and S249C positive UMUC-14 cells after 48 hours, at a timepoint when cytotoxicity activity was observed (See FIGS. 40A-F). Notably, the levels of cytokines induced by TCEs 1 and 3 and positive control R3Mabx38E4v1 were lower than those induced by Control E (FIGS. 40A-F).

### Example 12: In Vitro Cytotoxicity on HepG2 Cells

TCEs 1, 2, 3, 4 and 7 were assessed for cytotoxic activity on FGFR3IIIb wildtype expressing HepG2 cancer cells in the presence of human PBMCs.

Human PBMCs were purchased from BioIVT™. HepG2 was purchased from ATCC®.

PBMCs were thawed in RPMI+10% FBS and rested overnight before the experiment. On the day of the experiment, HepG2 target cells were harvested with TrypLE and washed with assay media (RPMI+10% FBS). PBMCs were also washed with assay media. Both PBMCs and target cells were counted and checked for viability with the cell counter ensuring that the viability is at least 80%. After cell counting, cells were resuspended in media for an E:T ratio of 10:1 by resuspending target cells at a concentration of 0.1 million cells/ml and the effector cells at a concentration of 2 million cells/ml. Next, 50 μl (5000) target cells were seeded into a 96 well plate followed by adding 25 μl (50,000) effector cells to each well. TCEs were titrated in a separate 96 well U-bottom plate at a start concentration of 10 μg/ml with a 1-to-3 dilution. 25 μl of titrated TCE was added in duplicate to wells containing effector and target cells. R3Mabx38E4v1 was titrated in the first 2 rows of each plate as a positive control with a start concentration of 1 μg/ml. Before incubating plates, 2 μl of KILR total lysis control solution was added to designated maximal lysis control wells. Plates were incubated at 37° C. for 48 hours. Before developing plates, plates were washed with assay media. CellTiter-Glo® Luminescent Cell Viability was assessed by equilibrating plates at room temperature for approximately 30 minutes before adding 80 μl CellTiter-Glo® solution to each well. The plates were gently shaken on a shaker for at least 3 minutes to allow complete cell lysis. Luminescence was measured with a luminometer at an integration of 500 ms.

Percent lysis values were calculated as follows: Percent Lysis=(Luminescence value of well-No TCE control)/Total Lysis control×100. Data was analyzed using GraphPad Prism version 10.1.2.

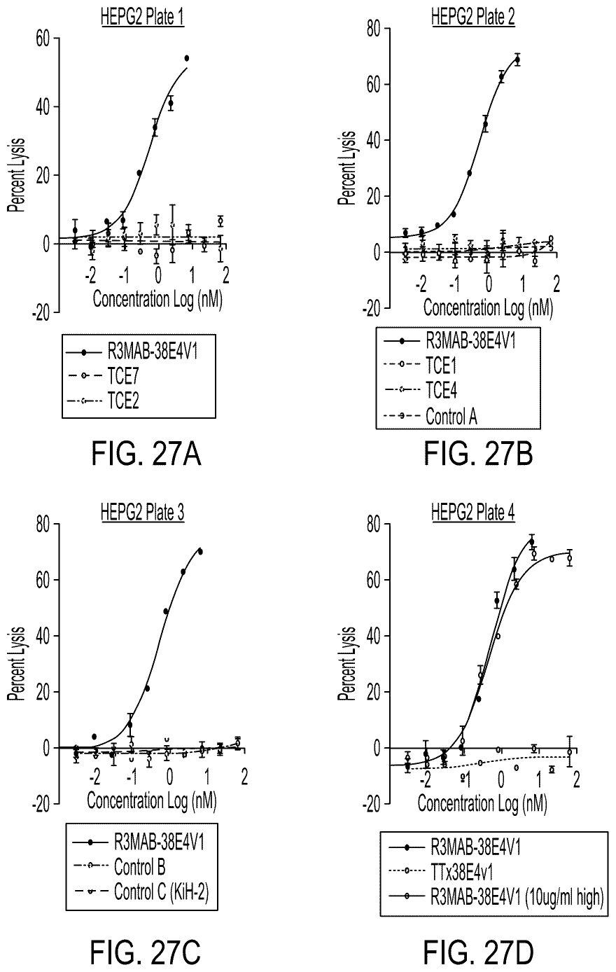

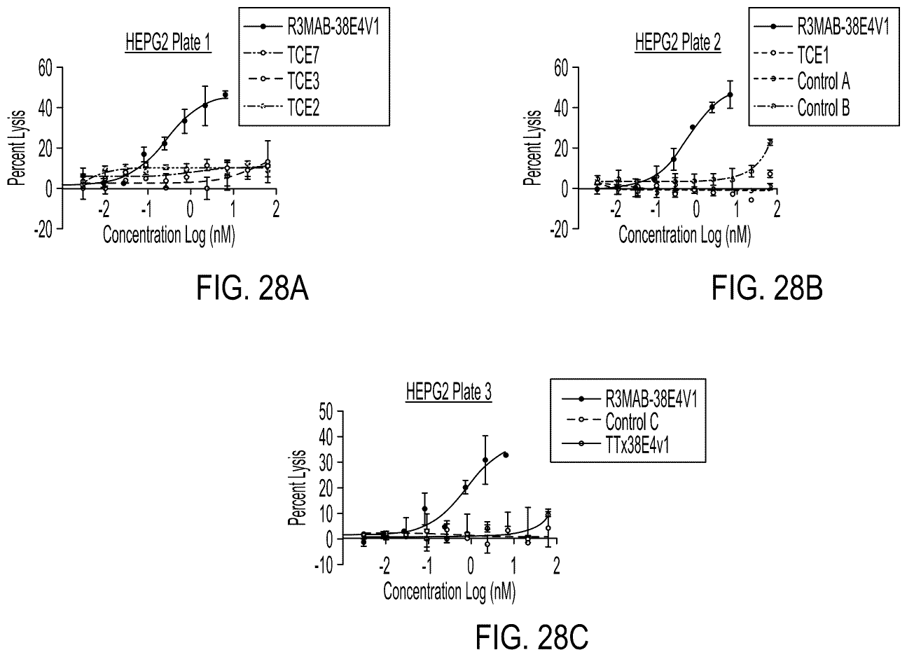

Summary: In the presence of human PBMCs in a 10:1 effector-to-target cell ratio, all TCEs of the invention tested demonstrated no cytotoxicity to the FGFR3IIIb wildtype expressing human cancer cell line, HepG2 (FIGS. 27A-B and 28A-B) when compared to negative Controls A, B and C as well as TT×38E4v1 (FIGS. 27C-D and 28B-C). In contrast, the positive control, R3Mabx38E4v1 consistently showed cytotoxicity of FGFR3IIIb WT HepG2 cells (FIGS. 27A-D and 28A-C).

### Example 13: In Vitro Cytotoxicity on OMC-1 Cells

TCEs 1, 2, 4, 5 and 7 were assessed for cytotoxic activity on endogenously expressing FGFR3IIIb S249C OMC-1 cervical cancer cells, in the presence of human expanded T cells.

Expanded T cells were generated from PBMC donor 10061 in house using conventional cell culture techniques. The OMC-1 cells were purchased from Riken BRC™.

On the day of the experiment, OMC-1 cells were harvested with TrypLE and washed 1× with Assay media (RPMI+10% FBS). The expanded T cells were thawed in RPMI+10% FBS and washed assay media with. Both effector and target cells were counted and checked for viability with the cell counter ensuring that the viability is at least 80%. After cell counting, cells were resuspended in media for an E: T ratio of 10:1 by resuspending target cells at a concentration of 0.1 million cells/ml and the effector cells at a concentration of 2 million cells/ml. Next, 50 μl (5000) target cells were seeded into a 96 well plate followed by adding 25 μl (50,000) effector cells to each well. TCEs were titrated in a separate 96 well U-bottom plate at a start concentration of 10 μg/ml with a 1-to-3 dilution. 25 μl of titrated TCE was added in duplicate to wells containing effector and target cells. R3Mabx38E4v1 was titrated in the first 2 rows of each plate as a positive control with a start concentration of 1 μg/ml. Before incubating plates, 2 μl of KILR total lysis control solution was added to designated maximal lysis control wells. Plates were incubated at 37° C. for 96 hours. Before developing plates, plates were washed with assay media. CellTiter-Glo® Luminescent Cell Viability was assessed by equilibrating plates at room temperature for approximately 30 minutes before adding 80 μl CellTiter-Glo® solution to each well. The plates were gently shaken on a shaker for at least 3 minutes to allow complete cell lysis. Luminescence was measured with a luminometer at an integration of 500 ms.

Percent lysis values were calculated as follows: Percent Lysis=(Luminescence value of well-No TCE control)/Total Lysis control×100. Data was analyzed using GraphPad Prism version 10.1.2.

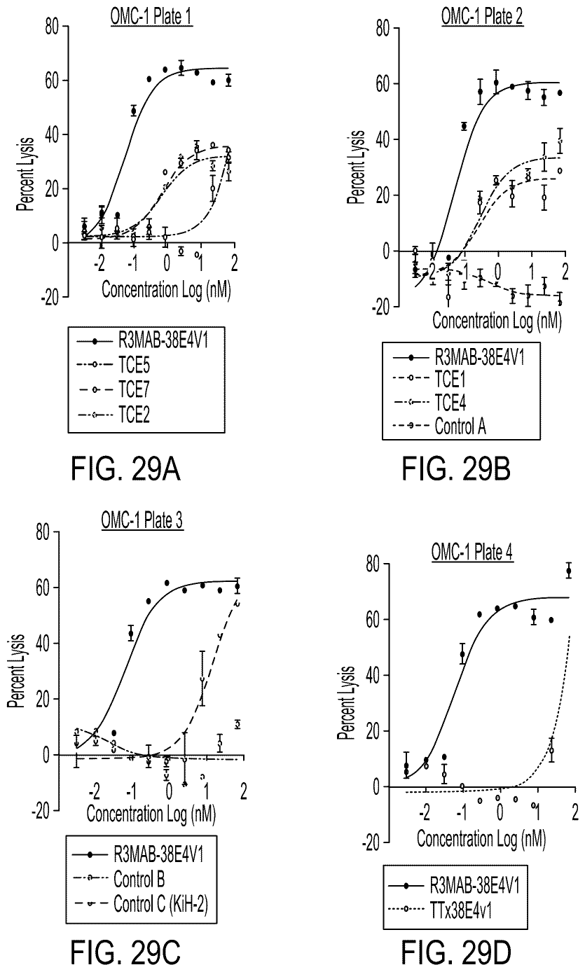

Summary: In a 10:1 effector-to-target cell ratio, all TCEs tested demonstrated cytotoxic activity against endogenously expressing FGFR3IIIb S249C OMC-1 human cervical cancer cells in the presence of human expanded T cells (FIG. 29A-B), while negative control TCEs showed minimal activity (FIG. 29C).

### Example 14: In Vivo Study in Human CD3edg Knock-In Mice

The anti-tumor activity of TCEs 1, 2, 3, 4 and 7 were evaluated in two separate studies using a transgenic mouse model utilizing homozygous human CD3edg knock-in mice implanted with murine breast cancer cells E0771 engineered to express human FGFR3IIIb S249C (TCEs 1, 2, 4 and 7) or with EO771-huFGFR3IIIb WT cells (TCEs 1 and 3).

The E0771 murine breast cancer cell line was obtained from C_{H}3 Biosystems™ (Product: #94A001). EO771-hFGFR3IIIb S249C and E0771-huFGFR3IIIb WT cells were both engineered using conventional molecular cloning techniques. Both cell lines were maintained in Dulbecco's Modified Eagle Medium (DMEM), Catalog No. 11995-081, supplemented 10% fetal bovine serum (Cat #SH300070.03) with 20 mM HEPES (Cat #15630-080) and 2 μg/ml Puromycin (Cat #A1113803). All cultures were maintained in a humidified incubator at 37° C. under 5% CO2/95% air, free of mycoplasma and pathogenic human and murine viruses. Following recovery from frozen stocks, low cell passages (up to 4) were used in the experiment. Human PBMCs were isolated from whole blood (Donor B001000834) using Ficoll-Hypaque density-gradient centrifugation and then frozen at −196° C. until use. All the test TCEs were diluted freshly for each dosing to a final concentration as indicated below in PBS. Homozygous hCD3edg knock-in mice (Strain No. 110039) were obtained from Biocytogen™ (Beijing).

On Day 0, 3×10⁵ E0771-FGFR3IIIb S249C murine breast cells were injected subcutaneously into right flank of mice (200 μl/mouse, in Hank's Balanced Salt Solution). On study Day 1 after cell implantation, the mice were randomized into groups at 5 mice/group and treated with vehicle (PBS) or test TCEs by body weight (10 μL/g). TCEs were administered at 0.44 mg/kg or 0.15 mg/kg or 0.05 mg/kg or 0.017 mg/kg as indicated below by intravenous injection (IV) once a week for three weeks. Tumor volume (TV) and body weight were measured twice per week. TV was calculated as TV (mm³)=T/6*length*width². Animals were sacrificed due to progressive disease if tumor burden was greater than 2000 mm³, or growth would surpass 2000 mm³ before the next measurement.

| EO771-huFGFR3IIIb S249C | EO771-huFGFR3IIIb WT |
|---|---|
| 5 mice per Group | 5 mice per Group |
| Group 1: Vehicle | Group 1: Vehicle |
| Group 2: 0.44 mg/kg TCE7-QW × 3 doses | Group 2: 0.44 mg/kg Control A QW × 3 doses |
| Group 3: 0.44 mg/kg TCE2-QW × 3 doses | Group 3: 0.44 mg/kg TCE1 QW × 3 doses |
| Group 4: 0.44 mg/kg TCE1-QW × 3 doses | Group 4: 0.15 mg/kg TCE1 QW × 3 doses |
| Group 5: 0.44 mg/kg TCE4-QW × 3 doses | Group 5: 0.05 mg/kg TCE1 QW × 3 doses |
| Group 6: 0.44 mg/kg Control B-QW × 3 doses | Group 6: 0.017 mg/kg TCE1 QW × 3 doses |
| Group 7: 0.44 mg/kg Control A-QW × 3 doses | Group 7: 0.44 mg/kg TCE3 QW × 3 doses |
| Group 8: 0.44 mg/kg Control C-QW × 3 doses | Group 8: 0.15 mg/kg TCE3 QW × 3 doses |
| Group 9: 0.44 mg/kg Control D-QW × 3 doses | Group 9: 0.05 mg/kg TCE3 QW × 3 doses |
| Group 10: 1 mg/kg R3Mabx38E4v1, QW × 3 | Group 10: 0.017 mg/kg TCE3 QW × 3 doses |
| doses | Group 11: 0.44 mg/kg Control C QW × 3 doses |
| | Group 12: 0.44 mg/kg R3Mabx38E4v1 QW × |
| | 3 doses |

Blood samples were collected retro-orbitally at 24 hrs post the first dose to measure cytokine levels in serum separator tube (BD sku #365967). Adequate time was allowed for blood to clot (~20 minutes) at room temperature, then blood was centrifuge at 10,000×g for 10 mins in a refrigerated centrifuge. Serum was collected, aliquoted and stored in −80° C.

Efficacy was calculated at the end of the treatment if the number of remaining control group subjects was at least half the baseline sample size or greater than 4. Otherwise, efficacy was calculated on the most recent observation day prior to the end of treatment where such conditions were met. Precent treatment/control (% T/C) were calculated as follows:

%T/C=100×ΔT/ΔC if ΔT>0

% Regression=100×ΔT/Tᵢₙᵢₜᵢₐₗ if ΔT<0 where:
T=mean tumor volume of the drug-treated group on the final day of the study;
ΔT=mean tumor volume of the drug-treated group on the final day of the study-mean tumor volume of the drug-treated group on initial day of dosing;
Tᵢₙᵢₜᵢₐₗ=mean tumor volume of the drug-treated group on initial day of dosing;
C=mean tumor volume of the control group on the final day of the study; and
AC=mean tumor volume of the control group on the final day of the study-mean tumor volume of the control group on initial day of dosing.

The % change in body weight (BW) was calculated as: (BW current-BW initial)/(BW initial)×100, where the data is presented as the % BW change from the day of treatment initiation.

Tables 7 and 8 show the quantification of certain results based on an N=5, where a p-value <0.05 is considered statistically significant; NA=Not Applicable; BL Day=Baseline Day.

TABLE 7
Anti-Tumor Efficacy in EO771-
huFGFR3IIIb S249C Tumors in hCD3edg Mice

| TCE/ | | | | |
|---|---|---|---|---|
| Days after initial treatment | | p-value | % T/C | Regression % |
| TCE1 | Day 0 | >0.999 | BL Day | BL Day |
| | Day 10 | <0.001 | 0.0 | 0.0 |
| | Day 14 | <0.001 | 1.2 | NA |
| | Day 17 | <0.001 | 2.6 | NA |
| | Day 21 | <0.001 | 6.1 | NA |
| TCE2 | Day 0 | >0.999 | BL Day | BL Day |
| | Day 10 | 0.067 | 38.4 | NA |
| | Day 14 | 0.022 | 34.8 | NA |
| | Day 17 | 0.049 | 42.1 | NA |
| | Day 21 | 0.219 | 59.3 | NA |
| TCE4 | Day 0 | >0.999 | BL Day | BL Day |
| | Day 10 | 0.015 | 26.3 | NA |
| | Day 14 | 0.117 | 49.4 | NA |
| | Day 17 | 0.135 | 52.2 | NA |
| | Day 21 | 0.348 | 67.2 | NA |
| TCE7 | Day 0 | >0.999 | BL Day | BL Day |
| | Day 10 | <0.001 | 5.4 | NA |
| | Day 14 | <0.001 | 8.4 | NA |
| | Day 17 | <0.001 | 10.1 | NA |
| | Day 21 | <0.001 | 8.9 | NA |
| Control A | Day 0 | >0.999 | BL Day | BL Day |
| | Day 10 | 0.913 | 94.9 | NA |
| | Day 14 | 0.578 | 78.2 | NA |
| | Day 17 | 0.695 | 84.5 | NA |
| | Day 21 | 0.845 | 92.1 | NA |
| Control B | Day 0 | >0.999 | BL Day | BL Day |
| | Day 10 | 0.281 | 58.4 | NA |
| | Day 14 | 0.607 | 79.7 | NA |
| | Day 17 | 0.514 | 75.6 | NA |
| | Day 21 | 0.616 | 80.9 | NA |
| Control C | Day 0 | >0.999 | BL Day | BL Day |
| | Day 10 | 0.499 | 71.8 | NA |
| | Day 14 | 0.789 | 88.9 | NA |
| | Day 17 | 0.641 | 81.9 | NA |
| | Day 21 | 0.843 | 92.0 | NA |
| Control D | Day 0 | >0.999 | BL Day | BL Day |
| | Day 10 | <0.001 | 8.4 | NA |
| | Day 14 | 0.002 | 22.0 | NA |
| | Day 17 | 0.006 | 29.2 | NA |
| | Day 21 | 0.024 | 37.7 | NA |
| R3MAbx38E4v1 | Day 0 | >0.999 | BL Day | BL Day |
| | Day 10 | <0.001 | 5.8 | NA |
| | Day 14 | <0.001 | 12.1 | NA |
| | Day 17 | <0.001 | 11.7 | NA |
| | Day 21 | <0.001 | 10.9 | NA |

TABLE 8
Anti-Tumor Efficacy in EO771-
huFGFR3IIIb WT Tumors in hCD3edg Mice

| TCE/ | | | | |
|---|---|---|---|---|
| Days after initial treatment | | p-value | % T/C | Regression % |
| TCE1 | Day 0 | >0.999 | BL Day | BL Day |
| 0.44 mg/kg | Day 10 | 0.181 | 145.4 | NA |
| | Day 13 | 0.268 | 77.4 | NA |
| | Day 16 | 0.206 | 75.3 | NA |
| | Day 20 | 0.118 | 71.0 | NA |
| | Day 23 | 0.363 | 82.1 | NA |
| | Day 27 | 0.075 | 68.1 | NA |
| TCE1 | Day 0 | >0.999 | BL Day | BL Day |
| 0.15 mg/kg | Day 10 | 0.026 | 184.1 | NA |
| | Day 13 | 0.962 | 98.9 | NA |
| | Day 16 | 0.512 | 86.4 | NA |
| | Day 20 | 0.887 | 96.9 | NA |
| | Day 23 | 0.878 | 103.4 | NA |
| | Day 27 | 0.921 | 102.2 | NA |
| TCE1 | Day 0 | >0.999 | BL Day | BL Day |
| 0.05 mg/kg | Day 10 | 0.004 | 217.5 | NA |
| | Day 13 | 0.339 | 124.2 | NA |
| | Day 16 | 0.592 | 112.6 | NA |
| | Day 20 | 0.638 | 110.8 | NA |
| | Day 23 | 0.627 | 111.1 | NA |
| | Day 27 | 0.343 | 122.6 | NA |
| TCE1 | Day 0 | >0.999 | BL Day | BL Day |
| 0.017 mg/kg | Day 10 | 0.769 | 108.8 | NA |
| | Day 13 | 0.845 | 95.6 | NA |
| | Day 16 | 0.719 | 92.3 | NA |
| | Day 20 | 0.669 | 91.1 | NA |
| | Day 23 | 0.774 | 94.0 | NA |
| | Day 27 | 0.565 | 88.4 | NA |
| Control A | Day 0 | >0.999 | BL Day | BL Day |
| 0.44 mg/kg | Day 10 | 0.007 | 207.6 | NA |
| | Day 13 | 0.900 | 97.2 | NA |
| | Day 16 | 0.797 | 105.9 | NA |
| | Day 20 | 0.898 | 102.8 | NA |
| | Day 23 | 0.611 | 111.6 | NA |
| | Day 27 | 0.765 | 93.8 | NA |
| TCE3 | Day 0 | >0.999 | BL Day | BL Day |
| 0.44 mg/kg | Day 10 | 0.252 | 138.1 | NA |
| | Day 13 | 0.629 | 89.5 | NA |
| | Day 16 | 0.442 | 84.2 | NA |
| | Day 20 | 0.393 | 83.0 | NA |
| | Day 23 | 0.190 | 75.3 | NA |
| | Day 27 | 0.724 | 92.7 | NA |
| TCE3 | Day 0 | >0.999 | BL Day | BL Day |
| 0.15 mg/kg | Day 10 | 0.024 | 185.1 | NA |
| | Day 13 | 0.774 | 106.8 | NA |
| | Day 16 | 0.663 | 90.7 | NA |
| | Day 20 | 0.350 | 81.5 | NA |
| | Day 23 | 0.370 | 82.3 | NA |
| | Day 27 | 0.253 | 78.2 | NA |
| TCE3 | Day 0 | >0.999 | BL Day | BL Day |
| 0.05 mg/kg | Day 10 | <0.001 | 278.4 | NA |
| | Day 13 | 0.931 | 98.0 | NA |
| | Day 16 | 0.831 | 95.4 | NA |
| | Day 20 | 0.762 | 93.6 | NA |
| | Day 23 | 0.792 | 94.5 | NA |
| | Day 27 | 0.911 | 97.6 | NA |
| TCE3 | Day 0 | >0.999 | BL Day | BL Day |
| 0.017 mg/kg | Day 10 | <0.001 | 242.4 | NA |
| | Day 13 | 0.581 | 113.4 | NA |
| | Day 16 | 0.873 | 96.5 | NA |
| | Day 20 | 0.954 | 101.3 | NA |
| | Day 23 | 0.810 | 105.3 | NA |
| | Day 27 | 0.840 | 104.4 | NA |
| Control C | Day 0 | >0.999 | BL Day | BL Day |
| | Day 10 | 0.021 | 188.0 | NA |
| | Day 13 | 0.539 | 115.0 | NA |
| | Day 16 | 0.896 | 97.1 | NA |
| | Day 20 | 0.958 | 98.9 | NA |
| | Day 23 | 0.909 | 102.5 | NA |
| | Day 27 | 0.804 | 105.5 | NA |
| R3MAbx38E4v1 | Day 0 | >0.999 | BL Day | BL Day |
| 0.44 mg/kg | Day 10 | 0.150 | 149.5 | NA |
| | Day 13 | 0.095 | 67.9 | NA |
| | Day 16 | 0.028 | 60.9 | NA |
| | Day 20 | 0.029 | 61.9 | NA |
| | Day 23 | 0.085 | 68.7 | NA |
| | Day 27 | 0.041 | 64.2 | NA |

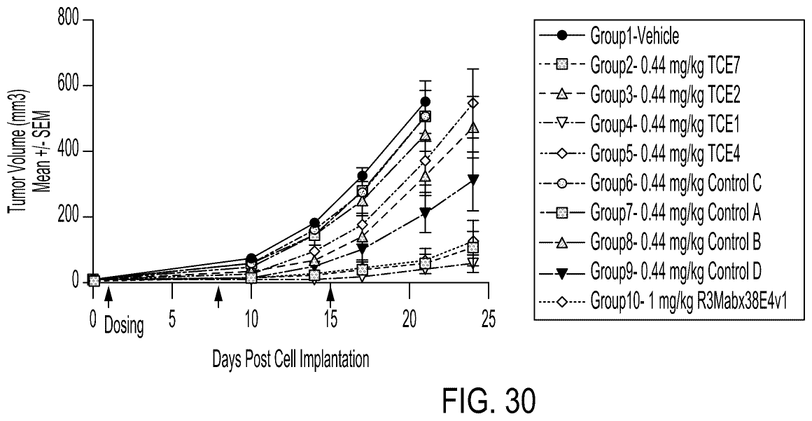

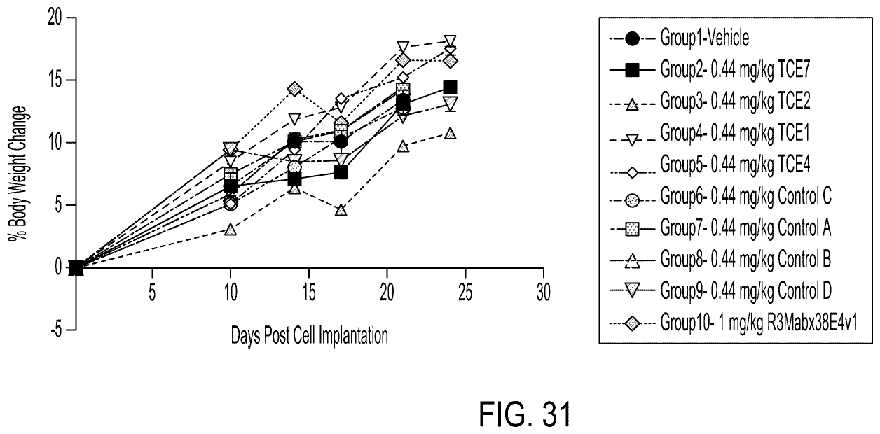

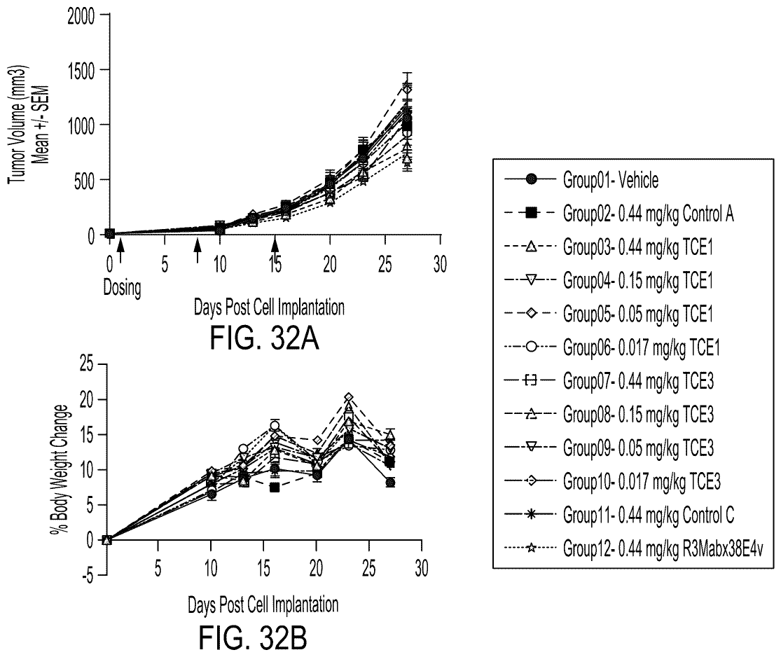

Summary: TCEs 1 and 7 significantly inhibited E0771-hFGFR3IIIb S249C tumor growth (p<0.001) on Day 21 to the same level as R3Mabx38E4v1 positive control, when compared to Vehicle and to Control D (FIG. 30 and Table 7). TCEs 2 and 4 inhibited tumor growth, but not as significantly (FIG. 30 and Table 7). Among the four TCEs tested, TCE1 showed the lowest percent treatment to control (T/C) rate (Table 7). Tumors in the three negative controls, Controls A, B and C, all had very similar growth rates as compared to Vehicle (FIG. 30 and Table 7). No significant body weight loss was observed in any group (FIG. 31). In the EO7771-hFGFR3IIIb WT model, none of the TCEs of the invention tested inhibited tumor growth at 0.44 mg/kg and 0.15 mg/kg when compared to Vehicle or negative Controls A or C (FIG. 32A and Table 8). Positive control R3Mabx38E4v1 showed the most anti-tumor activity as compared to TCEs 1 or 3 across all doses tested (p=0.041 compared to the Vehicle group, see FIG. 32A and Table 8) but this was expected given that R3Mab specifically binds FGFR3 WT. Body weight was not impacted by treatment with the TCEs (FIG. 32B). These results suggest that the TCEs of the invention have no effect on WT tumor growth in this xenograft model at the dose levels tested.

### Example 15: In Vivo Study in a Preventative Co-Implantation Xenograft Model

The anti-tumor activity of TCE 1, 2, 4 and 5 were evaluated in a preventative humanized xenograft model using overexpressing FGFR3IIIb S249C HeLa cell lines co-implanted with human PBMCs.

Female NOD SCID gamma (NSG) mice (Strain No. 005557) were obtained from Jackson Laboratory and acclimated for 1 week before initiating the experiment. Mice were housed in a 12-hour light/dark cycle facility under pathogen-free conditions in microisolator cages with standard laboratory chow and water ad libitum. Prepared Hela-FGFR3IIIb S249C cells [5×10⁶ with 1×10⁶ PBMCs in 200 μL, single-cell suspensions of over 90% viability in Hank's Balanced Saline Solution, mixed with an equal volume of Matrigel (200 μL)] were subcutaneously injected into the right hind flank of each mouse. On the next day, the mice were randomized into different groups.
A: Hela-FGFR3 S249C+PBMC, Vehicle.
B: Hela-FGFR3 S249C+PBMCs, 0.25 mg/kg TCE5-QW×2 doses
C: Hela-FGFR3 S249C+PBMCs, 0.25 mg/kg TCE2-QW×2 doses
D: Hela-FGFR3 S249C+PBMCs, 0.25 mg/kg TCE1-QW×2 doses
E: Hela-FGFR3 S249C+PBMCs, 0.25 mg/kg TCE4-QW×2 doses
F: Hela-FGFR3 S249C+PBMCs, 0.25 mg/kg Control C-QW×2 doses
G: Hela-FGFR3 S249C+PBMCs, 0.25 mg/kg Control A-QW×2 doses
H: Hela-FGFR3 S249C+PBMCs, 0.25 mg/kg Control B-QW×2 doses
I: Hela-FGFR3 S249C+PBMCs, 0.25 mg/kg R3Mabx38E4v1, QW×2 doses

Serum and blood samples were collected at 24 hrs post the first dose to test cytokine levels. Adequate time was allowed for blood to clot (~20 minutes) at room temperature, then blood was centrifuged at 10,000×g for 10 mins in a refrigerated centrifuge.

Efficacy was calculated as described above in Example 14. In addition, the % Delta T/C was defined as 100 times the ratio of the tumor volume change from Baseline at time t of the treated group versus the tumor volume change from Baseline of the control group at time t, where t is greater than tBaseline and the treated group change from Baseline is greater than zero. Baseline tumor volume is the grand mean of all tumors at tBaseline. The % Regression was defined as 100 times the ratio of the tumor volume change from Baseline of the treated group versus Baseline tumor volume at time t, where t is greater than tBaseline and the treated group change from Baseline is less than or equal to zero. The % Tumor Growth Inhibition (TGI) % TGI was defined as 100 minus % Delta T/C or 100 minus % Regression as applicable. Body weight and tumor volume were measured twice weekly. Tumor volume was calculated using the formula [tumor volume (mm³)=π/6× length×width²) and plotted as geometric means±SEM. The % T/C (ratio between the tumor volume in the treated group and in the control group) was calculated by the formula 100×ΔT/ΔC, if ΔT>0 of the geometric mean values. ΔT, mean tumor volume of the drug-treated group on the observation day of the study-mean tumor volume of the drug-treated group on initial day of dosing; ΔC, mean tumor volume of the control group on the observation day of the study—mean tumor volume of the control group on initial day of dosing. Regression was calculated using the formula=100×ΔT/Tᵢₙᵢₜᵢₐₗ, if ΔT<0. Animals with <14 mm³ tumor volume for three consecutive measurements were considered as complete responders and tumors with >50% regressions were partial responders. The % change in body weight was calculated by the formula (body weight on observation day−body weight on initial day)/body weight on initial day×100%.

Table 9 shows the quantification of certain results based on an N=5, where a p-value <0.05 is considered statistically significant; NA=Not Applicable; BL Day=Baseline Day.

TABLE 9
Anti-Tumor Efficacy in Hela-
FGFR3IIIb S249C Co-Implantation Model

| TCE/ | | | | |
|---|---|---|---|---|
| Days after initial treatment | | p-value | % T/C | Regression % |
| TCE1 | Day 7 | 0.749 | BL Day | BL Day |
| | Day 10 | 0.313 | NA | −21.2 |
| | Day 14 | 0.060 | NA | −18.8 |
| | Day 17 | <0.001 | NA | −63.1 |
| | Day 21 | <0.001 | NA | −40.1 |
| | Day 24 | <0.001 | NA | −70.5 |
| TCE2 | Day 7 | 0.969 | BL Day | BL Day |
| | Day 10 | 0.319 | NA | −5.8 |
| | Day 14 | 0.035 | NA | −9.5 |
| | Day 17 | <0.001 | NA | −56.2 |
| | Day 21 | <0.001 | NA | −82.5 |
| | Day 24 | <0.001 | NA | −86.6 |
| TCE4 | Day 7 | 0.960 | BL Day | BL Day |
| | Day 10 | 0.207 | NA | −21.6 |
| | Day 14 | <0.001 | NA | −80.1 |
| | Day 17 | <0.001 | NA | −88.0 |
| | Day 21 | <0.001 | NA | −87.8 |
| | Day 24 | <0.001 | NA | −89.4 |
| TCE5 | Day 7 | 0.836 | BL Day | BL Day |
| | Day 10 | 0.131 | NA | −18.0 |
| | Day 14 | <0.001 | NA | −75.8 |
| | Day 17 | <0.001 | NA | −75.5 |
| | Day 21 | <0.001 | NA | −74.1 |
| | Day 24 | <0.001 | NA | −81.5 |
| Control A | Day 7 | 0.768 | BL Day | BL Day |
| | Day 10 | 0.473 | 196.9 | NA |
| | Day 14 | 0.442 | 168.3 | NA |
| | Day 17 | 0.419 | 167.7 | NA |
| | Day 21 | 0.415 | 163.4 | NA |
| Control B | Day 7 | 0.596 | BL Day | BL Day |
| | Day 10 | 0.763 | 90.1 | NA |
| | Day 14 | 0.888 | 69.7 | NA |
| | Day 17 | 0.942 | 80.6 | NA |
| | Day 21 | 0.769 | 77.4 | NA |
| | Day 24 | 0.657 | 131.0 | NA |
| Control C | Day 7 | 0.885 | BL Day | BL Day |
| | Day 10 | 0.960 | 93.2 | NA |
| | Day 14 | 0.508 | 160.8 | NA |
| | Day 17 | 0.314 | 195.3 | NA |
| | Day 21 | 0.397 | 207.9 | NA |
| R3MAbx38E4v1 | Day 7 | 0.719 | BL Day | BL Day |
| | Day 10 | 0.837 | 34.0 | NA |
| | Day 14 | 0.319 | 23.1 | NA |
| | Day 17 | 0.923 | 83.9 | NA |
| | Day 21 | 0.161 | 35.0 | NA |
| | Day 24 | 0.954 | 81.0 | NA |

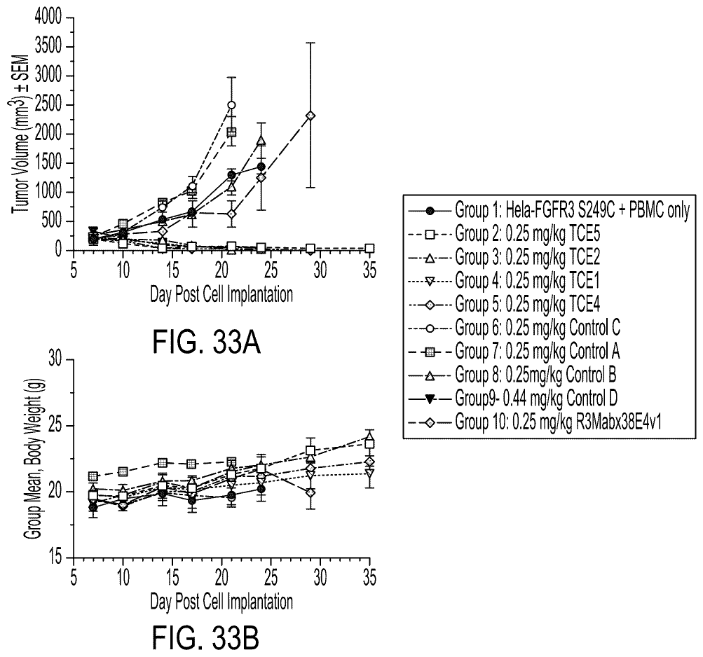

Summary: All TCEs tested at a 0.25 mg/kg dose (administered once weekly for 2 weeks) demonstrated anti-tumor efficacy by delaying tumor growth in a preventative xenograft model which led to complete tumor regression in a HeLa-FGFR3IIIb S249C tumor mouse model co-implanted with human PBMCs (FIG. 33A and Table 9). Tumors in dosed with Controls A, B and C had very similar growth rate with the vehicle group (FIG. 33A and Table 9). No significant body weight loss was observed in any group (FIG. 33B).

### Example 16: In Vivo Study in Xenograft Models

The anti-tumor activity of TCEs of the invention were evaluated in either a human bladder cancer UMUC-14 xenograft model that endogenously expresses the S249C mutation (TCEs 1, 2, 3, 4 and 7) or a human bladder cancer RT-112 xenograft model that endogenously expresses a WT-like FGFR3-TACC3 extracellular domain (TCEs 1 and 3), each with human PBMCs co-implanted.

Female NOD SCID gamma (NSG) mice (Strain No. 005557) were obtained from Jackson Laboratory and acclimated for one week before initiating the experiment. Mice were housed in a 12-hour light/dark cycle facility under pathogen-free conditions in microisolator cages with standard laboratory chow and water ad libitum.

On Day 0, either 2×10⁶ of UMUC-14 or RT-112 human bladder cells were mixed with 0.2×10⁶ or 0.625×10⁶ healthy human PBMCs, respectively, and injected subcutaneously into right flank of mice (200 μl/mouse, in Hank's Balanced Salt Solution with an equal volume of Matrigel). On study Day 1 after the cell implantation, NSG mice were randomized into treatment groups and treated with vehicle or test articles by body weight (10 μL/g). Different doses, described below, were injected by intravenous injection (IV) once a week for three weeks.

UMUC-14 Xenograft

| Experiment #1 | Experiment #2 |
|---|---|
| Group 1: Vehicle | Group 1: Vehicle |
| Group 2: 0.44 mg/kg TCE7-QW × 3 doses | Group 2: 0.44 mg/kg Control A-QW × 3 doses |
| Group 3: 0.44 mg/kg TCE2-QW × 3 doses | Group 3: 0.44 mg/kg TCE1-QW × 3 doses |
| Group 4: 0.44 mg/kg TCE1-QW × 3 doses | Group 4: 0.15 mg/kg TCE1-QW × 3 doses |
| Group 5: 0.44 mg/kg TCE4-QW × 3 doses | Group 5: 0.05 mg/kg TCE1-QW × 3 doses |
| Group 6: 0.44 mg/kg Control C-QW × 3 doses | Group 6: 0.017 mg/kg TCE1-QW × 3 doses |
| Group 7: 0.44mg/kg Control A-QW × 3 doses | Group 7: 0.44 mg/kg Control C-QW × 3 doses |
| Group 8: 0.44 mg/kg Control B-QW × 3 doses | Group 8: 0.44 mg/kg TCE3-QW × 3 doses |
| Group 9: 0.44 mg/kg Control D-QW × 3 doses | Group 9: 0.15 mg/kg TCE3-QW × 3 doses |
| Group 10: 1 mg/kg R3Mabx38E4v1, QW × 3 | Group 10: 0.05 mg/kg TCE3-QW × 3 doses |
| doses | Group 11: 0.017 mg/kg TCE3 QW × 3 doses |
| | Group 12: 1 mg/kg R3Mabx38E4v1, QW × 3 |
| | doses |

RT-112 Xenograft

Group 1: Vehicle
Group 2: 0.44mg/kg Control A-QW × 3 doses
Group 3: 0.44 mg/kg TCE1-QW × 3 doses
Group 4: 0.15 mg/kg TCE1-QW × 3 doses
Group 5: 0.05 mg/kg TCE1-QW × 3 doses
Group 6: 0.017 mg/kg TCE1-QW × 3 doses
Group 7: 0.44 mg/kg Control C-QW × 3 doses
Group 8: 0.44 mg/kg TCE3-QW × 3 doses
Group 9: 0.15 mg/kg TCE3-QW × 3 doses
Group 10: 0.05 mg/kg TCE3-QW × 3 doses
Group 11: 0.017 mg/kg TCE3-QW × 3 doses
Group 12: 1 mg/kg R3Mabx38E4v1, QW × 3 doses

Tumor volume and body weight were measured twice per week. TV was calculated as TV (mm³)=π/6*length*width². Animals were sacrificed due to progressive disease if tumor burden was greater than 2000 mm³, or growth would surpass 2000 mm³ before the next measurement.

Blood samples from both xenograft studies were collected retro-orbitally at 24 hrs post the first dose (3 mice/group) to measure cytokine levels in serum separator tube (BD sku #365967). Adequate time was allowed for blood to clot (~20 minutes) at room temperature, then blood was centrifuged at 10,000×g for 10 mins in a refrigerated centrifuge. Serum was collected, aliquoted and stored in-80° C.

Tables 10, 11 and 12 show the quantification of certain results of each experiment performed based on an N=6 for Tables 10 and 11 and N=5 for Table 12, where a p-value <0.05 is considered statistically significant; NA=Not Applicable; BL Day=Baseline Day.

TABLE 10
Experiment #1 in UMUC-14 Xenograft Model

| TCE/ | | | | |
|---|---|---|---|---|
| Days after initial treatment | | p-value | % T/C | Regression % |
| TCE1 | Day 0 | >0.999 | BL Day | BL Day |
| 0.44 mg/kg | Day 8 | 0.058 | 71.0 | NA |
| | Day 12 | <0.001 | 52.3 | NA |
| | Day 15 | <0.001 | 46.8 | NA |
| | Day 19 | <0.001 | 33.5 | NA |
| | Day 23 | <0.001 | 26.4 | NA |
| | Day 27 | <0.001 | 22.9 | NA |
| | Day 33 | <0.001 | 21.0 | NA |
| | Day 36 | <0.001 | 22.1 | NA |
| TCE2 | Day 0 | >0.999 | BL Day | BL Day |
| 0.44 mg/kg | Day 8 | 0.178 | 78.6 | NA |
| | Day 12 | 0.002 | 56.8 | NA |
| | Day 15 | 0.001 | 55.3 | NA |
| | Day 19 | <0.001 | 42.3 | NA |
| | Day 23 | <0.001 | 43.5 | NA |
| | Day 27 | <0.001 | 46.4 | NA |
| | Day 33 | <0.001 | 50.3 | NA |
| | Day 36 | <0.001 | 51.5 | NA |
| TCE4 | Day 0 | >0.999 | BL Day | BL Day |
| 0.44 mg/kg | Day 8 | 0.180 | 78.7 | NA |
| | Day 12 | 0.010 | 62.6 | NA |
| | Day 15 | 0.002 | 56.7 | NA |
| | Day 19 | <0.001 | 42.1 | NA |
| | Day 23 | <0.001 | 34.6 | NA |
| | Day 27 | <0.001 | 31.6 | NA |
| | Day 33 | <0.001 | 32.2 | NA |
| | Day 36 | <0.001 | 35.7 | NA |
| TCE7 | Day 0 | >0.999 | BL Day | BL Day |
| 0.44 mg/kg | Day 8 | 0.230 | 80.7 | NA |
| | Day 12 | <0.001 | 50.3 | NA |
| | Day 15 | <0.001 | 42.8 | NA |
| | Day 19 | <0.001 | 33.1 | NA |
| | Day 23 | <0.001 | 33.2 | NA |
| | Day 27 | <0.001 | 31.2 | NA |
| | Day 33 | <0.001 | 38.7 | NA |
| | Day 36 | <0.001 | 40.7 | NA |
| Control A | Day 0 | >0.999 | BL Day | BL Day |
| 0.44 mg/kg | Day 8 | 0.959 | 100.9 | NA |
| | Day 12 | 0.890 | 102.5 | NA |
| | Day 15 | 0.689 | 93.3 | NA |
| | Day 19 | 0.382 | 86.1 | NA |
| | Day 23 | 0.305 | 83.9 | NA |
| | Day 27 | 0.688 | 93.4 | NA |
| | Day 33 | 0.832 | 103.6 | NA |
| | Day 36 | 0.508 | 113.5 | NA |
| Control B | Day 0 | >0.999 | BL Day | BL Day |
| 0.44 mg/kg | Day 8 | 0.943 | 98.8 | NA |
| | Day 12 | 0.940 | 98.7 | NA |
| | Day 15 | 0.674 | 92.9 | NA |
| | Day 19 | 0.372 | 85.8 | NA |
| | Day 23 | 0.500 | 89.1 | NA |
| | Day 27 | 0.450 | 88.0 | NA |
| | Day 33 | 0.398 | 86.7 | NA |
| | Day 36 | 0.231 | 82.1 | NA |
| Control C | Day 0 | >0.999 | BL Day | BL Day |
| 0.44 mg/kg | Day 8 | 0.965 | 99.2 | NA |
| | Day 12 | 0.944 | 101.2 | NA |
| | Day 15 | 0.692 | 107.1 | NA |
| | Day 19 | 0.924 | 101.7 | NA |
| | Day 23 | 0.923 | 101.7 | NA |
| | Day 27 | 0.926 | 101.6 | NA |
| | Day 33 | 0.740 | 94.6 | NA |
| | Day 36 | 0.636 | 93.0 | NA |
| Control D | Day 0 | >0.999 | BL Day | BL Day |
| 0.44 mg/kg | Day 8 | 0.279 | 82.5 | NA |
| | Day 12 | 0.008 | 61.8 | NA |
| | Day 15 | 0.001 | 55.6 | NA |
| | Day 19 | <0.001 | 43.0 | NA |
| | Day 23 | <0.001 | 40.0 | NA |
| | Day 27 | <0.001 | 41.1 | NA |
| | Day 33 | <0.001 | 50.6 | NA |
| | Day 36 | <0.001 | 55.1 | NA |
| R3MAbx38E4v1 | Day 0 | >0.999 | BL Day | BL Day |
| 1 mg/kg | Day 8 | 0.302 | 83.2 | NA |
| | Day 12 | 0.002 | 55.8 | NA |
| | Day 15 | <0.001 | 52.1 | NA |
| | Day 19 | <0.001 | 37.5 | NA |
| | Day 23 | <0.001 | 34.0 | NA |
| | Day 27 | <0.001 | 23.6 | NA |
| | Day 33 | <0.001 | 31.6 | NA |
| | Day 36 | <0.001 | 32.6 | NA |

TABLE 11
Experiment #2 in UMUC-14 Xenograft Model

| TCE/ | | | | |
|---|---|---|---|---|
| Days after initial treatment | | p-value | % T/C | Regression % |
| TCE1 | Day 9 | 0.014 | BL Day | BL Day |
| 0.44 mg/kg | Day 12 | <0.001 | NA | −43.4 |
| | Day 16 | <0.001 | NA | −38.2 |
| | Day 19 | <0.001 | NA | −54.0 |
| | Day 26 | <0.001 | 1.6 | NA |
| | Day 29 | NA | NA | NA |
| | Day 33 | NA | NA | NA |
| | Day 37 | NA | NA | NA |
| | Day 40 | NA | NA | NA |
| TCE1 | Day 9 | 0.011 | BL Day | BL Day |
| 0.15 mg/kg | Day 12 | <0.001 | NA | −57.6 |
| | Day 16 | <0.001 | NA | −56.6 |
| | Day 19 | <0.001 | NA | −60.4 |
| | Day 26 | <0.001 | NA | −32.5 |
| | Day 29 | NA | NA | NA |
| | Day 33 | NA | NA | NA |
| | Day 37 | NA | NA | NA |
| | Day 40 | NA | NA | NA |
| TCE1 | Day 9 | 0.314 | BL Day | BL Day |
| 0.05 mg/kg | Day 12 | 0.029 | NA | −29.1 |
| | Day 16 | 0.011 | NA | −25.4 |
| | Day 19 | <0.001 | NA | −75.1 |
| | Day 26 | <0.001 | NA | −81.2 |
| | Day 29 | NA | NA | NA |
| | Day 33 | NA | NA | NA |
| | Day 37 | NA | NA | NA |
| | Day 40 | NA | NA | NA |
| TCE1 | Day 9 | 0.533 | BL Day | BL Day |
| 0.017 mg/kg | Day 12 | 0.330 | 28.2 | NA |
| | Day 16 | 0.91 | 2.4 | NA |
| | Day 19 | <0.001 | NA | −66.1 |
| | Day 26 | 0.003 | 2.5 | NA |
| | Day 29 | NA | NA | NA |
| | Day 33 | NA | NA | NA |
| | Day 37 | NA | NA | NA |
| | Day 40 | NA | NA | NA |
| Control A | Day 9 | 0.727 | BL Day | BL Day |
| 0.44 mg/kg | Day 12 | 0.567 | 49.0 | NA |
| | Day 16 | 0.559 | 56.7 | NA |
| | Day 19 | 0.128 | 11.3 | NA |
| | Day 26 | 0.713 | 77.1 | NA |
| | Day 29 | NA | NA | NA |
| TCE3 | Day 9 | 0.567 | BL Day | BL Day |
| 0.44 mg/kg | Day 12 | 0.138 | NA | −14.1 |
| | Day 16 | 0.048 | NA | −17.6 |
| | Day 19 | 0.50 | 3.3 | NA |
| | Day 26 | 0.342 | 48.6 | NA |
| | Day 29 | NA | NA | NA |
| | Day 33 | NA | NA | NA |
| TCE3 | Day 9 | 0.329 | BL Day | BL Day |
| 0.15 mg/kg | Day 12 | 0.073 | NA | −8.6 |
| | Day 16 | 0.118 | 19.3 | NA |
| | Day 19 | 0.012 | NA | −2.0 |
| | Day 26 | 0.011 | 10.6 | NA |
| | Day 29 | NA | NA | NA |
| | Day 33 | NA | NA | NA |
| | Day 37 | NA | NA | NA |
| | Day 40 | NA | NA | NA |
| TCE3 | Day 9 | 0.328 | BL Day | BL Day |
| 0.05 mg/kg | Day 12 | 0.041 | NA | −23.2 |
| | Day 16 | 0.038 | 0.9 | NA |
| | Day 19 | <0.001 | NA | −54.7 |
| | Day 26 | <0.001 | NA | −55.7 |
| | Day 29 | NA | NA | NA |
| | Day 33 | NA | NA | NA |
| | Day 37 | NA | NA | NA |
| | Day 40 | NA | NA | NA |
| TCE | Day 9 | 0.331 | BL Day | BL Day |
| 0.017 mg/kg | Day 12 | 0.402 | 63.5 | NA |
| | Day 16 | 0.357 | 54.0 | NA |
| | Day 19 | 0.060 | 14.1 | NA |
| | Day 26 | 0.773 | 87.3 | NA |
| Control C | Day 9 | 0.545 | BL Day | BL Day |
| 0.44 mg/kg | Day 12 | 0.441 | 47.0 | NA |
| | Day 16 | 0.756 | 92.7 | NA |
| | Day 19 | 0.864 | 100.8 | NA |
| | Day 26 | 0.922 | 114.6 | NA |
| R3MAbx38E4v1 | Day 9 | 0.388 | BL Day | BL Day |
| 1 mg/kg | Day 12 | 0.020 | NA | −42.5 |
| | Day 16 | 0.042 | NA | −3.1 |
| | Day 19 | 0.011 | NA | −11.0 |
| | Day 26 | 0.275 | 45.1 | NA |
| | Day 29 | NA | NA | NA |
| | Day 33 | NA | NA | NA |

TABLE 12
RT-112 Xenograft Model

| TCE/ | | | | |
|---|---|---|---|---|
| Days after initial treatment | | p-value | % T/C | Regression % |
| TCE1 | Day 0 | >0.999 | BL Day | BL Day |
| 0.44 mg/kg | Day 6 | 0.623 | 93.0 | NA |
| | Day 10 | 0.033 | 73.1 | NA |
| | Day 14 | <0.001 | 60.2 | NA |
| | Day 17 | <0.001 | 43.6 | NA |
| | Day 21 | <0.001 | 34.9 | NA |
| | Day 24 | <0.001 | 30.5 | NA |
| | Day 29 | <0.001 | 21.6 | NA |
| | Day 35 | <0.001 | 13.8 | NA |
| | Day 38 | <0.001 | 10.8 | NA |
| TCE1 | Day 0 | >0.999 | BL Day | BL Day |
| 0.15 mg/kg | Day 6 | 0.793 | 96.2 | NA |
| | Day 10 | 0.547 | 91.7 | NA |
| | Day 14 | 0.488 | 90.5 | NA |
| | Day 17 | 0.123 | 80.3 | NA |
| | Day 21 | 0.526 | 91.5 | NA |
| | Day 24 | 0.799 | 96.5 | NA |
| | Day 29 | 0.238 | 85.0 | NA |
| | Day 35 | <0.001 | 60.8 | NA |
| | Day 38 | <0.001 | 49.4 | NA |
| TCE1 | Day 0 | >0.999 | BL Day | BL Day |
| 0.05 mg/kg | Day 6 | 0.983 | 99.7 | NA |
| | Day 10 | 0.372 | 90.1 | NA |
| | Day 14 | 0.713 | 94.9 | NA |
| | Day 17 | 0.432 | 89.5 | NA |
| | Day 21 | 0.654 | 94.0 | NA |
| | Day 24 | 0.684 | 94.5 | NA |
| | Day 29 | 0.067 | 77.6 | NA |
| | Day 35 | <0.001 | 59.3 | NA |
| | Day 38 | <0.001 | 62.2 | NA |
| TCE1 | Day 0 | >0.999 | BL Day | BL Day |
| 0.017 mg/kg | Day 6 | 0.674 | 94.0 | NA |
| | Day 10 | 0.395 | 88.4 | NA |
| | Day 14 | 0.343 | 87.3 | NA |
| | Day 17 | 0.319 | 86.9 | NA |
| | Day 21 | 0.392 | 88.8 | NA |
| | Day 24 | 0.253 | 85.3 | NA |
| | Day 29 | 0.055 | 76.6 | NA |
| | Day 35 | <0.001 | 54.0 | NA |
| | Day 38 | <0.001 | 51.3 | NA |
| Control A | Day 0 | >0.999 | BL Day | BL Day |
| 0.44 mg/kg | Day 6 | 0.966 | 100.7 | NA |
| | Day 10 | 0.952 | 99.0 | NA |
| | Day 14 | 0.704 | 94.0 | NA |
| | Day 17 | 0.257 | 83.2 | NA |
| | Day 21 | 0.249 | 83.1 | NA |
| | Day 24 | 0.494 | 89.7 | NA |
| | Day 29 | 0.156 | 79.9 | NA |
| | Day 35 | 0.462 | 89.2 | NA |
| | Day 38 | 0.803 | 96.2 | NA |
| TCE7 | Day 0 | >0.999 | BL Day | BL Day |
| 0.44 mg/kg | Day 6 | 0.871 | 97.6 | NA |
| | Day 10 | 0.048 | 74.8 | NA |
| | Day 14 | <0.001 | 56.8 | NA |
| | Day 17 | <0.001 | 41.5 | NA |
| | Day 21 | <0.001 | 39.0 | NA |
| | Day 24 | <0.001 | 33.4 | NA |
| | Day 29 | <0.001 | 22.5 | NA |
| | Day 35 | <0.001 | 13.0 | NA |
| | Day 38 | <0.001 | 11.0 | NA |
| TCE7 | Day 0 | >0.999 | BL Day | BL Day |
| 0.15 mg/kg | Day 6 | 0.786 | 86.1 | NA |
| | Day 10 | 0.70 | 76.6 | NA |
| | Day 14 | 0.039 | 74.0 | NA |
| | Day 17 | 0.023 | 72.1 | NA |
| | Day 21 | 0.006 | 67.4 | NA |
| | Day 24 | 0.001 | 63.4 | NA |
| | Day 29 | <0.001 | 46.2 | NA |
| | Day 35 | <0.001 | 23.2 | NA |
| | Day 38 | <0.001 | 16.9 | NA |
| TCE7 | Day 0 | >0.999 | BL Day | BL Day |
| 0.05 mg/kg | Day 6 | 0.847 | 97.2 | NA |
| | Day 10 | 0.082 | 77.5 | NA |
| | Day 14 | 0.015 | 70.1 | NA |
| | Day 17 | 0.011 | 69.1 | NA |
| | Day 21 | 0.017 | 71.3 | NA |
| | Day 24 | 0.018 | 71.5 | NA |
| | Day 29 | 0.001 | 63.6 | NA |
| | Day 35 | <0.001 | 53.9 | NA |
| | Day 38 | <0.001 | 49.8 | NA |
| TCE7 | Day 0 | >0.999 | BL Day | BL Day |
| 0.017 mg/kg | Day 6 | 0.877 | 97.4 | NA |
| | Day 10 | 0.214 | 81.3 | NA |
| | Day 14 | 0.241 | 82.4 | NA |
| | Day 17 | 0.337 | 85.6 | NA |
| | Day 21 | 0.532 | 90.5 | NA |
| | Day 24 | 0.669 | 93.4 | NA |
| | Day 29 | 0.793 | 96.0 | NA |
| | Day 35 | 0.129 | 78.8 | NA |
| | Day 38 | 0.136 | 79.2 | NA |
| Control C | Day 0 | >0.999 | BL Day | BL Day |
| 0.44 mg/kg | Day 6 | 0.964 | 99.2 | NA |
| | Day 10 | 0.902 | 102.1 | NA |
| | Day 14 | 0.869 | 97.3 | NA |
| | Day 17 | 0.831 | 96.6 | NA |
| | Day 21 | 0.896 | 102.1 | NA |
| | Day 24 | 0.901 | 102.0 | NA |
| | Day 29 | 0.978 | 100.4 | NA |
| | Day 35 | 0.886 | 97.8 | NA |
| | Day 38 | 0.953 | 99.1 | NA |
| R3MAbx38E4v1 | Day 0 | >0.999 | BL Day | BL Day |
| 1 mg/kg | Day 6 | 0.771 | 95.8 | NA |
| | Day 10 | 0.001 | 61.6 | NA |
| | Day 14 | <0.001 | 54.3 | NA |
| | Day 17 | <0.001 | 38.1 | NA |
| | Day 21 | <0.001 | 26.3 | NA |
| | Day 24 | <0.001 | 21.9 | NA |
| | Day 29 | <0.001 | 13.9 | NA |
| | Day 35 | <0.001 | 6.9 | NA |
| | Day 38 | <0.001 | 5.8 | NA |

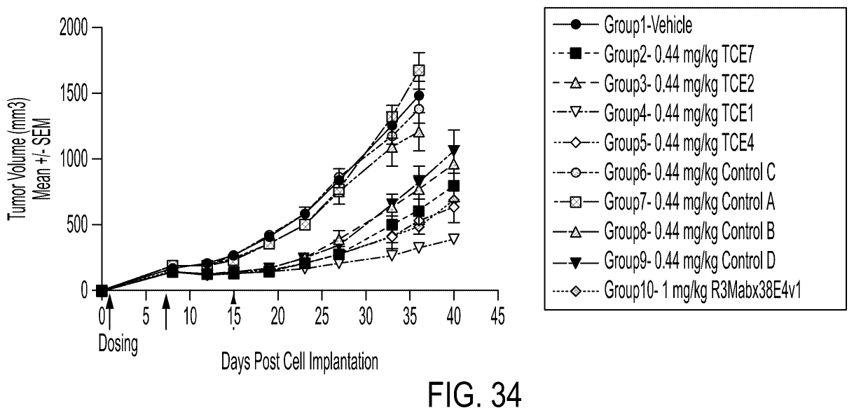

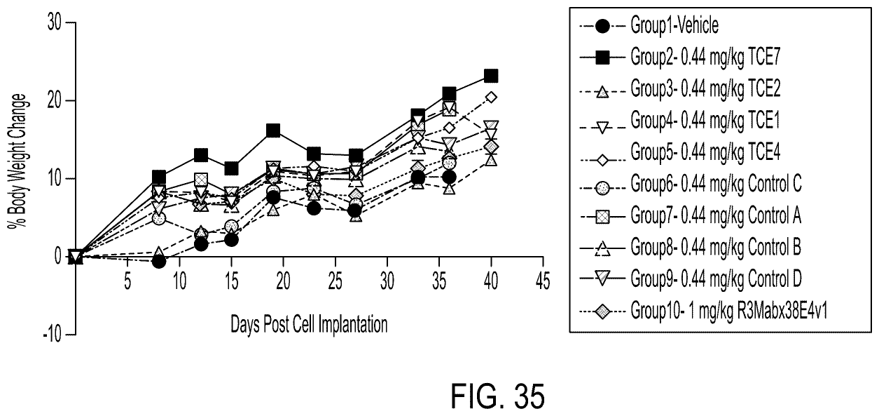

Experiment #1 UMUC-14 xenograft summary: All TCEs tested at a 0.44 mg/kg dose (administered once weekly for 3 weeks) demonstrated anti-tumor efficacy in a human bladder cancer UMUC-14 xenograft mouse model co-implanted with human PBMCs (FIG. 34). TCEs 1, 2, 4 and 7 and R3Mabx38E4v1 positive control all significantly inhibited UMUC-14 tumor growth (p<0.001) on Day 36 as compared to Vehicle group and Control D (FIG. 34 and Table 10). Among the TCEs tested, TCE1 showed the lowest T/C rate and greatest tumor growth delay, see Table 10. Tumors in all the negative control groups (Controls A, B and C) had very similar growth rate as compared with the Vehicle group. No significant body weight loss was observed in any group (FIG. 35).

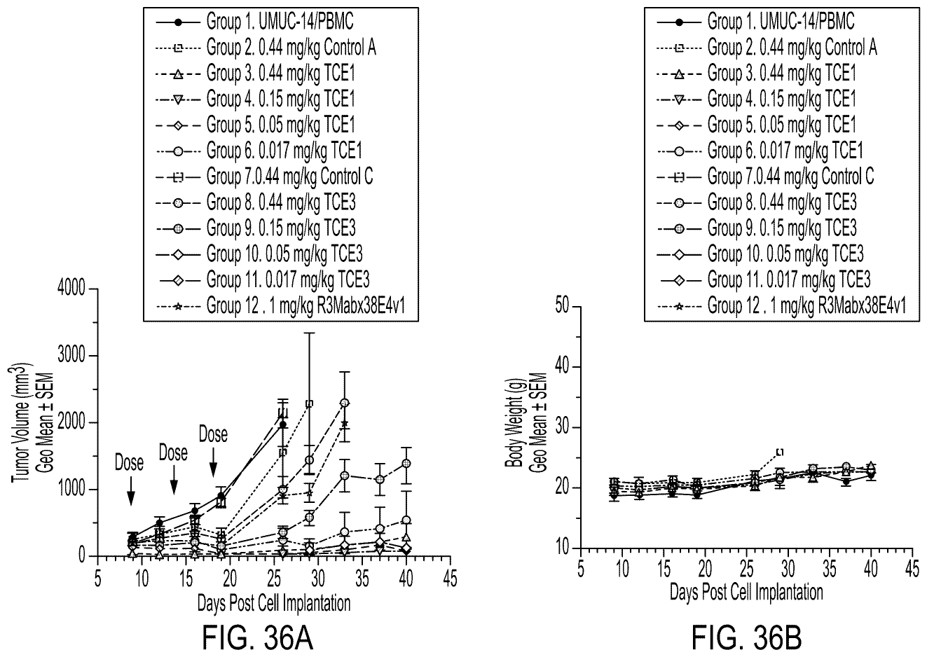

Experiment #2 UMUC-14 xenograft summary: This study demonstrated dose-dependent anti-tumor efficacy of TCEs 1 and 3 (FIG. 36A). TCE1 demonstrated an enhanced S249C+UMUC-14 tumor growth delay compared to TCE3 at the doses tested (FIG. 36A and Table 11). No significant body weight loss was observed in any group tested (FIG. 36B).

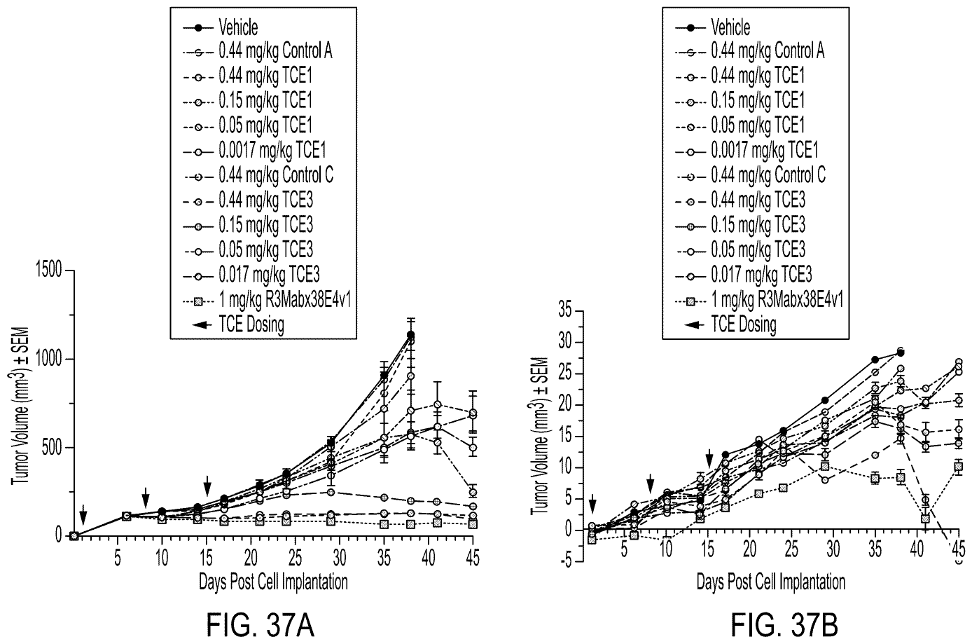

RT-112 xenograft summary: TCEs tested at a 0.44 mg/kg dose (administered once weekly for 3 weeks) demonstrated anti-tumor efficacy in a human bladder cancer RT-112 xenograft mouse model co-implanted with human PBMCs (FIG. 37A and Table 12). 0.44 mg/kg of both TCE1 and 3 significantly inhibited RT-112 tumor growth (P<0.001) on Day 38 compared with Vehicle (FIG. 37A and Table 12). Lower dose levels at 0.15 mg/kg, 0.05 mg/kg or 0.017 mg/kg did not significantly inhibit RT112 tumors. Tumors treated with Controls A and C had very similar growth rates to the Vehicle group (FIG. 37A and Table 12). No significant body weight loss was observed in any group (FIG. 37B).

### Example 17: Ex Vivo Analysis of Serum Cytokine Levels from Example 15

Serum cytokine levels induced by treatment of TCEs 1, 2 and 7 dosed at 0.44 mg/kg QWx3 in a preventative UMUC-14/PBMC co-implantation xenograft tumor model (see Example 16) were evaluated at 24 hours post treatment.

Two assays, an IL2 single-plex and a cytokine 10-plex (Quanterix™ #100-0487 and #85-0329, respectively), were used for this study. Assays were conducted per manufacturer's instruction. Briefly, assay reagents were taken out to room temperature for 30 minutes before start while samples were thawed on ice. Calibrators/standards were reconstituted with diluent and a seven-point serial dilution of the standards was done. Samples were prepared by adding 22 μl of serum to 33 μl of diluent. All washes were performed on the Bio Tek™ Plate Washer ELX406. Each assay plate was washed 10× with 300 μl of 1× washing buffer before loading of calibrators and samples. 50 μl of either calibrators or samples was added to the assay plate and shaken at ~525 rpm for 2 hours. Each assay plate was washed 10× with 300 μl of 1× washing buffer before adding 50 μl of biotinylated detection reagent and shaken for 30 minutes. Each plate was washed for another 10× with 300 μl of 1× washing buffer before adding 50 μl of Streptavidin-HRP and shaken for 30 minutes. SuperSignal™ reagents were added and the plate was immediately scanned on the SP-X™ Imager.

Assay was read on the SP-X™ Imager and data was analysis using the SP-X™ Analysis software, version 2.2.8789. Assay product information and sample plate layout with calibrator and sample dilution factors were loaded onto software prior to data analysis. Standard curve for each analyte was manually and minimally adjusted to ensure a curve fit close to 1. After confirming the standard curve for each analyte, quantification of each sample was calculated by the software. Sample concentration was exported to Excel and graphed on TIBCO Spotfire™ with the lower limit of quantification (LLOQ), limit of detection (LOD), and error bars representing the standard deviation of the average concentration per group.

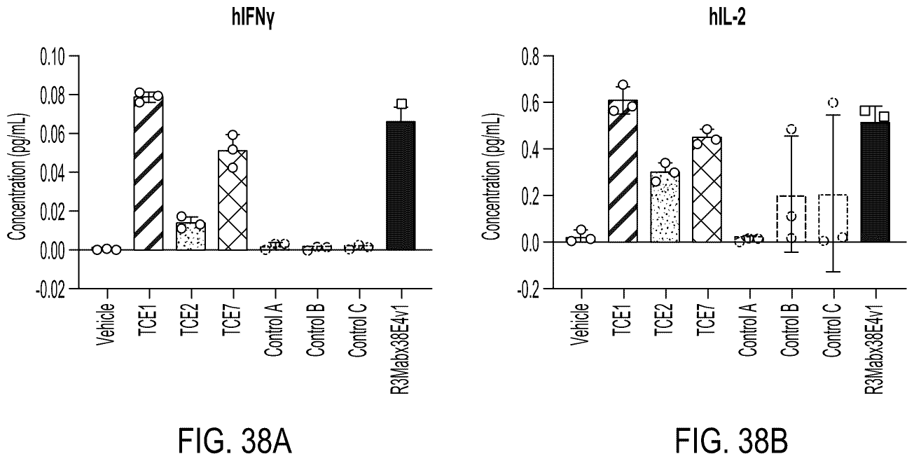

Summary: High levels of human IL-2 and human IFNg were detected for all TCEs tested. IFNg appeared to be induced the most by TCE1 followed by TCE7 and TCE2 (FIG. 38A). Similarly, IL-2 was induced the most by TCE1, followed by TCE7 and TCE2 (FIG. 38B). Overall, there was a high induction of IL-8 by all TCEs tested (data not shown). Levels of other 10-plex analytes (IL-1b, IL-4, IL-5, IL-6, IL-10, IL-12p70, IL-22, TNFa) were low and below the LLOQ (data not shown).

1. A bispecific antibody that specifically binds human fibroblast growth factor receptor 3 isoform IIIb serine 249 cysteine (hFGFR3IIIb S249C) and human CD3 (hCD3), wherein the bispecific antibody comprises (a) a first antigen binding domain that specifically binds hFGFR3IIIb S249C comprising a first heavy chain variable region (VH1) and a light chain variable region (VL1), wherein the VH1 comprises heavy chain complementarity determining regions (HCDR) HCDR1, HCDR2, and HCDR3, and the VL1 comprises light chain complementarity determining regions (LCDR) LCDR1, LCDR2, and LCDR3, wherein:
(a) HCDR1 comprises SEQ ID NO:1, HCDR2 comprises SEQ ID NO:2, HCDR3 comprises SEQ ID NO: 3, LCDR1 comprises SEQ ID NO:4, LCDR2 comprises SEQ ID NO:5, LCDR3 comprises SEQ ID NO: 6;
(b) HCDR1 comprises SEQ ID NO:1, HCDR2 comprises SEQ ID NO:7, HCDR3 comprises SEQ ID NO:8, LCDR1 comprises SEQ ID NO:4, LCDR2 comprises SEQ ID NO:5, LCDR3 comprises SEQ ID NO:6;
(c) HCDR1 comprises SEQ ID NO:9, HCDR2 comprises SEQ ID NO:10, HCDR3 comprises SEQ ID NO:11, LCDR1 comprises SEQ ID NO:4, LCDR2 comprises SEQ ID NO:5, LCDR3 comprises SEQ ID NO:6; or
(d) HCDR1 comprises SEQ ID NO:1, HCDR2 comprises SEQ ID NO:7, HCDR3 comprises SEQ ID NO:12, LCDR1 comprises SEQ ID NO:4, LCDR2 comprises SEQ ID NO:5, LCDR3 comprises SEQ ID NO:6,
and a second antigen binding domain that specifically binds human CD3.

2. The bispecific antibody of claim 1, wherein the second antigen binding domain that specifically binds hCD3 comprises a second heavy chain variable region (VH2) and a light chain variable region (VL2), wherein the VH2 comprises heavy chain complementarity determining regions (HCDR) HCDR4, HCDR5,
and HCDR6, and the VL2 comprises light chain complementarity determining regions (LCDR) LCDR4, LCDR5, and LCDR6.

3. A bispecific antibody that specifically binds hFGFR3IIIb S249C and hCD3, wherein the bispecific antibody comprises (a) a first antigen binding domain that specifically binds hFGFR3IIIb S249C and (b) a second antigen binding domain that specifically binds hCD3 comprising a heavy chain variable region (VH2) and a light chain variable region (VL2), wherein the VH2 comprises heavy chain complementarity determining regions (HCDR) HCDR4, HCDR5, and HCDR6, and the VL2 comprises light chain complementarity determining regions (LCDR) LCDR4, LCDR5, and LCDR6, wherein:
(a) HCDR4 comprises SEQ ID NO:13, HCDR5 comprises SEQ ID NO:14, HCDR6 comprises SEQ ID NO:15, LCDR4 comprises SEQ ID NO:4, LCDR5 comprises SEQ ID NO:5, LCDR6 comprises SEQ ID NO:6;
(b) HCDR4 comprises SEQ ID NO:16, HCDR5 comprises SEQ ID NO:17, HCDR6 comprises SEQ ID NO:18, LCDR4 comprises SEQ ID NO:4, LCDR5 comprises SEQ ID NO:5, LCDR6 comprises SEQ ID NO:6; or
(c) HCDR4 comprises SEQ ID NO:19, HCDR5 comprises SEQ ID NO:20, HCDR6 comprises SEQ ID NO:21, LCDR4 comprises SEQ ID NO:4, LCDR5 comprises SEQ ID NO:5, LCDR6 comprises SEQ ID NO:6.

4. The bispecific antibody of claim 1, wherein the bispecific antibody does not specifically bind to wild-type (WT) FGFR3.

5. The bispecific antibody of claim 1, wherein the bispecific antibody does not specifically bind to FGFR1, or FGFR2, or FGFR4.

6. The bispecific antibody of claim 1, wherein the bispecific antibody does not specifically bind to FGFR3 having the R248C mutation.

7. The bispecific antibody of claim 1, wherein the bispecific does not specifically bind to either wild-type (WT) or S249C mutant form of the FGFR3 isoform IIIc.

8. The bispecific antibody of claim 1, wherein the first antigen binding domain has about a 1.5-fold to 10-fold greater binding affinity (K_{D}) for hFGFR3IIIb S249C relative to the binding affinity the second antigen binding domain has for hCD3, as measured by surface plasmon resonance (SPR).

9. The bispecific antibody of claim 2, wherein:
(a) HCDR1 comprises SEQ ID NO:1, HCDR2 comprises SEQ ID NO: 2, HCDR3 comprises SEQ ID NO:3, LCDR1 comprises SEQ ID NO:4, LCDR2 comprises SEQ ID NO:5, LCDR3 comprises SEQ ID NO:6, HCDR4 comprises SEQ ID NO: 13, HCDR5 comprises SEQ ID NO: 14, HCDR6 comprises SEQ ID NO:15, LCDR4 comprises SEQ ID NO:4, LCDR5 comprises SEQ ID NO: 5, LCDR6 comprises SEQ ID NO:6;
(b) HCDR1 comprises SEQ ID NO:1, HCDR2 comprises SEQ ID NO: 2, HCDR3 comprises SEQ ID NO:3, LCDR1 comprises SEQ ID NO:4, LCDR2 comprises SEQ ID NO:5, LCDR3 comprises SEQ ID NO:6, HCDR4 comprises SEQ ID NO:16, HCDR5 comprises SEQ ID NO:17, HCDR6 comprises SEQ ID NO:18, LCDR4 comprises SEQ ID NO:4, LCDR5 comprises SEQ ID NO: 5, LCDR6 comprises SEQ ID NO: 6;
(c) HCDR1 comprises SEQ ID NO:1, HCDR2 comprises SEQ ID NO: 7, HCDR3 comprises SEQ ID NO:8, LCDR1 comprises SEQ ID NO:4, LCDR2 comprises SEQ ID NO:5, LCDR3 comprises SEQ ID NO:6, HCDR4 comprises SEQ ID NO: 19, HCDR5 comprises SEQ ID NO:20, HCDR6 comprises SEQ ID NO:21, LCDR4 comprises SEQ ID NO:4, LCDR5 comprises SEQ ID NO: 5, LCDR6 comprises SEQ ID NO:6;
(d) HCDR1 comprises SEQ ID NO:9, HCDR2 comprises SEQ ID NO: 10, HCDR3 comprises SEQ ID NO:11, LCDR1 comprises SEQ ID NO:4, LCDR2 comprises SEQ ID NO:5, LCDR3 comprises SEQ ID NO:6, HCDR4 comprises SEQ ID NO:16, HCDR5 comprises SEQ ID NO:17, HCDR6 comprises SEQ ID NO:18, LCDR4 comprises SEQ ID NO:4, LCDR5 comprises SEQ ID NO: 5, LCDR6 comprises SEQ ID NO:6;
(e) HCDR1 comprises SEQ ID NO:1, HCDR2 comprises SEQ ID NO: 2, HCDR3 comprises SEQ ID NO:3, LCDR1 comprises SEQ ID NO:4, LCDR2 comprises SEQ ID NO:5, LCDR3 comprises SEQ ID NO:6, HCDR4 comprises SEQ ID NO: 19, HCDR5 comprises SEQ ID NO:20, HCDR6 comprises SEQ ID NO:21, LCDR4 comprises SEQ ID NO:4, LCDR5 comprises SEQ ID NO: 5, LCDR6 comprises SEQ ID NO:6;
(f) HCDR1 comprises SEQ ID NO:9, HCDR2 comprises SEQ ID NO: 10, HCDR3 comprises SEQ ID NO:11, LCDR1 comprises SEQ ID NO:4, LCDR2 comprises SEQ ID NO:5, LCDR3 comprises SEQ ID NO:6, HCDR4 comprises SEQ ID NO: 19, HCDR5 comprises SEQ ID NO:20, HCDR6 comprises SEQ ID NO:21, LCDR4 comprises SEQ ID NO:4, LCDR5 comprises SEQ ID NO: 5, LCDR6 comprises SEQ ID NO:6; or
(g) HCDR1 comprises SEQ ID NO:1, HCDR2 comprises SEQ ID NO: 7, HCDR3 comprises SEQ ID NO:12, LCDR1 comprises SEQ ID NO:4, LCDR2 comprises SEQ ID NO:5, LCDR3 comprises SEQ ID NO:6, HCDR4 comprises SEQ ID NO:19, HCDR5 comprises SEQ ID NO:20, HCDR6 comprises SEQ ID NO:21, LCDR4 comprises SEQ ID NO:4, LCDR5 comprises SEQ ID NO: 5, LCDR6 comprises SEQ ID NO:6.

10. The bispecific antibody of claim 1, wherein:
(a) the VH1 comprises SEQ ID NO:22 and the VL1 comprises SEQ ID NO:23 and the VH2 comprises SEQ ID NO: 27 and the VL2 comprises SEQ ID NO:23;
(b) the VH1 comprises SEQ ID NO:22 and the VL1 comprises SEQ ID NO:23 and the VH2 comprises SEQ ID NO:28 and the VL2 comprises SEQ ID NO:23;
(c) the VH1 comprises SEQ ID NO:24 and the VL1 comprises SEQ ID NO: 23 and the VH2 comprises SEQ ID NO: 45 and the VL2 comprises SEQ ID NO:23;
(d) the VH1 comprises SEQ ID NO:25 and the VL1 comprises SEQ ID NO: 23 and the VH2 comprises SEQ ID NO:28 and the VL2 comprises SEQ ID NO:23;
(e) the VH1 comprises SEQ ID NO:22 and the VL1 comprises SEQ ID NO: 23 and the VH2 comprises SEQ ID NO:45 and the VL2 comprises SEQ ID NO:23;
(f) the VH1 comprises SEQ ID NO:25 and the VL1 comprises SEQ ID NO: 23 and the VH2 comprises SEQ ID NO:45 and the VL2 comprises SEQ ID NO:23; or
(g) the VH1 comprises SEQ ID NO:26 and the VL1 comprises SEQ ID NO: 23 and the VH2 comprises SEQ ID NO:28 and the VL2 comprises SEQ ID NO:23.

11. The bispecific antibody of claim 1, further comprising an Fe region comprising (a) an FGFR3IIIb S249C heavy chain constant region comprising a first C_{H}2 and a first C_{H}3 domains and (b) a CD3 heavy chain constant region comprising a second C_{H}2 and a second C_{H}3 domains, wherein the first C_{H}3 domain has amino acid substitutions L351D and L368E and the second C_{H}3 domain has amino acid substitutions L351K and T366K, according to the Kabat numbering system.

12. The bispecific antibody of claim 1, further comprising an Fe region comprising (a) an FGFR3IIIb S249C heavy chain constant region comprising a first C_{H}2 and a first C_{H}3 domains and (b) a CD3 heavy chain constant region comprising a second C_{H}2 and a second C_{H}3 domains, wherein the first C_{H}3 domain has amino acid substitutions T366S/L368A/Y407V and the second C_{H}3 domain has amino acid substitution T366W, according to the Kabat numbering system.

13. The bispecific antibody of claim 11, wherein the Fc region comprises Fc silencing mutations.

14. The bispecific antibody of claim 13, wherein the Fc silencing mutations are (a) L235G and G236R or (b) L234A, L235A or (c) L234A, L235A and D265A or (d) L234A, L235A and P329G or (e) N297A or (f) N297A and K322A or (g) L234A, L235A and D265S, each according to the Kabat numbering system.

15. The bispecific antibody of claim 1, wherein the antibody comprises a first heavy chain (HC1) that specifically binds human FGFR3IIIb S249C; a second heavy chain (HC2) that specifically binds human CD3; and a common light chain (cLC) wherein:
(a) HC1 comprises SEQ ID NO:32, HC2 comprises SEQ ID NO:33, CLC comprises SEQ ID NO: 37;
(b) HC1 comprises SEQ ID NO:32, HC2 comprises SEQ ID NO:39, cLC comprises SEQ ID NO: 37;
(c) HC1 comprises SEQ ID NO:34, HC2 comprises SEQ ID NO:38, cLC comprises SEQ ID NO: 37;
(d) HC1 comprises SEQ ID NO:35, HC2 comprises SEQ ID NO:39, cLC comprises SEQ ID NO: 37;
(e) HC1 comprises SEQ ID NO:32, HC2 comprises SEQ ID NO:38, cLC comprises SEQ ID NO: 37;
(f) HC1 comprises SEQ ID NO:35, HC2 comprises SEQ ID NO:38, cLC comprises SEQ ID NO: 37; or
(g) HC1 comprises SEQ ID NO:36, HC2 comprises SEQ ID NO:38, cLC comprises SEQ ID NO: 37.

16. The bispecific antibody of claim 1, wherein the antibody is a human IgG1 or IgG4 isotype.

17. The bispecific antibody of claim 16, wherein the antibody is a human IgG1 isotype.

18. A nucleic acid encoding the amino acid sequences of claim 15.

19. A host cell transfected with:
a) a first vector comprising the nucleic acids encoding SEQ ID NO: 32, and a second vector comprising the nucleic acids encoding SEQ ID NO:33;
b) a first vector comprising the nucleic acids encoding SEQ ID NO: 32, and a second vector comprising the nucleic acids encoding SEQ ID NO:39;
c) a first vector comprising the nucleic acids encoding SEQ ID NO: 34, and a second vector comprising the nucleic acids encoding SEQ ID NO:38;
d) a first vector comprising the nucleic acids encoding SEQ ID NO: 35, and a second vector comprising the nucleic acids encoding SEQ ID NO:39;
e) a first vector comprising the nucleic acids encoding SEQ ID NO: 32, and a second vector comprising the nucleic acids encoding SEQ ID NO:38;
f) a first vector comprising the nucleic acids encoding SEQ ID NO: 35, and a second vector comprising the nucleic acids encoding SEQ ID NO:38;
g) a first vector comprising the nucleic acids encoding SEQ ID NO: 36, and a second vector comprising the nucleic acids encoding SEQ ID NO:38.

20. The host cell of claim 19, further transfected with a third vector comprising the nucleic acids encoding SEQ ID NO: 37.

21. The host cell of claim 19, wherein the host cell is a mammalian host cell.

22. A process of producing a bispecific antibody comprising culturing the cell of claim 21 in a culture medium under conditions such that the bispecific antibody is expressed, and then recovering the bispecific antibody from the culture medium.

23. A pharmaceutical composition comprising the hFGFR3IIIb S249C×hCD3 bispecific antibody of claim 1, and a pharmaceutically acceptable excipient, diluent or carrier.

24. An isolated antibody that specifically binds hFGFR3IIIb S249C, wherein the antibody comprises a heavy chain variable region (VH) and a light chain variable region (VL), wherein the VH comprises heavy chain complementarity determining regions (HCDR): HCDR1, HCDR2, and HCDR3, and the VL comprises light chain complementarity determining regions (LCDR): LCDR1, LCDR2, and LCDR3, wherein:
(a) HCDR1 comprises SEQ ID NO:1, HCDR2 comprises SEQ ID NO:2, HCDR3 comprises SEQ ID NO:3, LCDR1 comprises SEQ ID NO:4, LCDR2 comprises SEQ ID NO:5, LCDR3 comprises SEQ ID NO:6;
(b) HCDR1 comprises SEQ ID NO:1, HCDR2 comprises SEQ ID NO:7, HCDR3 comprises SEQ ID NO:8, LCDR1 comprises SEQ ID NO:4, LCDR2 comprises SEQ ID NO:5, LCDR3 comprises SEQ ID NO:6;
(c) HCDR1 comprises SEQ ID NO:9, HCDR2 comprises SEQ ID NO:10, HCDR3 comprises SEQ ID NO:11, LCDR1 comprises SEQ ID NO:4, LCDR2 comprises SEQ ID NO:5, LCDR3 comprises SEQ ID NO:6; or
(d) HCDR1 comprises SEQ ID NO:1, HCDR2 comprises SEQ ID NO:7, HCDR3 comprises SEQ ID NO:12, LCDR1 comprises SEQ ID NO:4, LCDR2 comprises SEQ ID NO:5, LCDR3 comprises SEQ ID NO:6.

25. The antibody of claim 24, wherein:
(a) the VH comprises SEQ ID NO: 22 and the VL comprises SEQ ID NO: 23;
(b) the VH comprises SEQ ID NO: 24 and the VL comprises SEQ ID NO:23;
(c) the VH comprises SEQ ID NO: 25 and the VL comprises SEQ ID NO: 23; or
(d) the VH comprises SEQ ID NO: 26 and the VL comprises SEQ ID NO: 23.

26. The antibody of claim 24, wherein the antibody comprises a heavy chain (HC) and a light chain (LC), wherein:
(a) the HC comprises SEQ ID NO:40 and the LC comprises SEQ ID NO:37;
(b) the HC comprises SEQ ID NO:41 and the LC comprises SEQ ID NO:37;
(c) the HC comprises SEQ ID NO:42 and the LC comprises SEQ ID NO:37; or
(d) the HC comprises SEQ ID NO:43 and the LC comprises SEQ ID NO:37.

27. The antibody of claim 24, wherein the antibody is a human IgG1 or IgG4 isotype.

28. The antibody of claim 27, wherein the antibody is a human IgG1 isotype.

29. The antibody of claim 24, wherein the antibody is an antibody fragment or antigen-binding fragment.

30. The antibody of claim 29, wherein the antibody fragment or antigen-binding fragment is a Fab, a Fab′, an F(ab′) 2, a single-chain variable fragment (scFv), an Fv, a disulfide-linked Fv (sdFv), an Fd fragment, or a single-chain Fab (scFab).

31. The antibody of claim 24, wherein the Fc region comprises Fc silencing mutations.

32. The antibody of claim 31, wherein the Fc silencing mutations are (a) L235G and G236R or (b) L234A, L235A or (c) L234A, L235A and D265A or (d) L234A, L235A and P329G or (e) N297A or (f) N297A and K322A or (g) L234A, L235A and D265S, each according to the Kabat numbering system.

33. The antibody of claim 24, wherein the antibody is a multispecific antibody.

34. The antibody of claim 33, wherein the multispecific antibody is a bispecific antibody, or a trispecific antibody, or a tetraspecific antibody, or a diabody, or a tandem scFv, or a tandem VHH, or a tandem scFab.

35. An antibody-drug conjugate (ADC) comprising the antibody of claim 24 and a drug moiety.

36. The ADC of claim 35, wherein the drug moiety is selected from the group consisting of auristatin, N-acetyl-y calicheamicin, maytansinoid, pyrrolobenzodiazepine, exatecan and SN-38.

37. An immunocytokine comprising the antibody of claim 24 and a cytokine.

38. The immunocytokine of claim 37, wherein the cytokine is selected from the group consisting of IL-2, IL-4, IL-10, IL-12, IL-15, TNF, and IFNa.

39. A chimeric antigen receptor (CAR) comprising the antibody of claim 24, a transmembrane domain, and an intracellular signaling domain.

40. A pharmaceutical composition comprising the hFGFR3IIIb S249C antibody of claim 24 and a pharmaceutically acceptable excipient, diluent or carrier.

41. A method of treating an FGFR3IIIb S249C positive cancer in a subject in need thereof, comprising administering to the subject an effective amount of the bispecific antibody of claim 1.

42. The method of claim 41, wherein the FGFR3IIIb S249C positive cancer is relapsed or refractory.

43. The method of claim 41, wherein the FGFR3IIIb S249C positive cancer is metastatic.

44. The method of claim 41, wherein the FGFR3I11b S249C positive cancer is bladder cancer, non-muscle invasive bladder cancer (NMIBC), muscle invasive bladder cancer (MIBC), urothelial carcinoma, papillary urothelial carcinomas, infiltrating renal pelvis and ureter urothelial carcinoma, urethral urothelial carcinoma, urinary tract cancer, renal cell carcinoma, colorectal carcinoma, pancreatic exocrine carcinoma, lung cancer, including non-small cell lung cancer (NSCLC) and squamous cell lung carcinoma, skin cancer, cervical cancer, ovarian cancer, endometrial adenocarcinoma, HPV-positive vulvar squamous cell carcinoma (VSCC), prostate cancer, gastric adenocarcinoma, esophageal cancer, upper aero-digestive tract (UAT) cancer, glioma, and head and neck squamous cell carcinoma.

45. The method of claim 41, wherein the pharmaceutical composition induces an in vivo human IFNγ concentration of about 0.10 pg/ml or less.

46. The method of claim 41, wherein the pharmaceutical composition induces an in vivo human IL-2 concentration of about 0.6 pg/ml or less.

47.-55. (canceled)

56. A pharmaceutical composition comprising the hFGFR3IIIb S249C antibody of claim 24 and a pharmaceutically acceptable excipient, diluent or carrier.

57. A pharmaceutical composition comprising the ADC of claim 35 and a pharmaceutically acceptable excipient, diluent or carrier.

58. A pharmaceutical composition comprising the immunocytokine of claim 37 and a pharmaceutically acceptable excipient, diluent or carrier.

59. A pharmaceutical composition comprising the CAR of claim 39 and a pharmaceutically acceptable excipient, diluent or carrier.

60. A method of treating an FGFR3IIIb S249C positive cancer in a subject in need thereof, comprising administering to the subject an effective amount of the antibody of claim 24.

61. A method of treating an FGFR3IIIb S249C positive cancer in a subject in need thereof, comprising administering to the subject an effective amount of the ADC of claim 35.

62. A method of treating an FGFR3IIIb S249C positive cancer in a subject in need thereof, comprising administering to the subject an effective amount of the immunocytokine of claim 37.

63. A method of treating an FGFR3IIIb S249C positive cancer in a subject in need thereof, comprising administering to the subject an effective amount of the CAR of claim 39.


SEQUENCE LISTING (45 sequences)

SEQ ID NO: 1 - protein, 15 aa, synthetic construct
TFSGFSLSTSGEGVG

SEQ ID NO: 2 - protein, 9 aa, synthetic construct
LIYWTDDKR

SEQ ID NO: 3 - protein, 20 aa, synthetic construct
AHRRGGYSDHDSYYYYYLDV

SEQ ID NO: 4 - protein, 11 aa, synthetic construct
RASQSISSYLN

SEQ ID NO: 5 - protein, 8 aa, synthetic construct
YAASSLQS

SEQ ID NO: 6 - protein, 9 aa, synthetic construct
QQSYSTPPT

SEQ ID NO: 7 - protein, 9 aa, synthetic construct
LIYWSDDKR

SEQ ID NO: 8 - protein, 20 aa, synthetic construct
AHRRGGYSDHDSYYYYYMDV

SEQ ID NO: 9 - protein, 15 aa, synthetic construct
TFSGFSLSTSGEAVG

SEQ ID NO: 10 - protein, 9 aa, synthetic construct
LIYWSDDSR

SEQ ID NO: 11 - protein, 18 aa, synthetic construct
AHRRGSYSGDYYYYYMDV

SEQ ID NO: 12 - protein, 20 aa, synthetic construct
AHRRGGYSGYDSYYYYYMDV

SEQ ID NO: 13 - protein, 15 aa, synthetic construct
KASGDAFKSKTFTIS

SEQ ID NO: 14 - protein, 10 aa, synthetic construct
GIIPVFGTIT

SEQ ID NO: 15 - protein, 11 aa, synthetic construct
TRRGKWNPFDP

SEQ ID NO: 16 - protein, 13 aa, synthetic construct
KASGGTFRSFGIS

SEQ ID NO: 17 - protein, 10 aa, synthetic construct
GFIPVLGTAN

SEQ ID NO: 18 - protein, 11 aa, synthetic construct
ARRGNWNPFDP

SEQ ID NO: 19 - protein, 13 aa, synthetic construct
ATSGFTFDDYTMH

SEQ ID NO: 20 - protein, 9 aa, synthetic construct
ISWSSGSIG

SEQ ID NO: 21 - protein, 17 aa, synthetic construct
AKDHRGYGDYEGGGFDH

SEQ ID NO: 22 - protein, 128 aa, synthetic construct
QITLKESGPTLVKPTQTLTLTCTFSGFSLSTSGEGVGWIRQPPGKALEWLALIYWTDDKRYSPSLKSRLTITKDTSKNHVVLTMTNMDPVDTATYYCAHRRGGYSDHDSYYYYYLDVWGKGTTVTVSS

SEQ ID NO: 23 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 24 - protein, 128 aa, synthetic construct
QITLKESGPTLVKPTQTLTLTCTFSGFSLSTSGEGVGWIRQPPGKALEWLALIYWSDDKRYSPSLKNRLTITKDTSKNQVVLTMTNMDPVDTATYYCAHRRGGYSDHDSYYYYYMDVWGKGTMVTVSS

SEQ ID NO: 25 - protein, 126 aa, synthetic construct
QITLKESGPTLVKPTQTLTLTCTFSGFSLSTSGEAVGWIRQPPGKALEWLALIYWSDDSRYSPSLKSRLTITRVTAKNQVVLKMTNMDPVDTATYYCAHRRGSYSGDYYYYYMDVWGKGTMVTVSS

SEQ ID NO: 26 - protein, 128 aa, synthetic construct
RITLKESGPTLVKPTQTLTLTCTFSGFSLSTSGEGVGWIRQPPGKALEWLALIYWSDDKRYSPSLKNRLTITKDTSKNQVVLTMTNMDPVDTATYYCAHRRGGYSGYDSYYYYYMDVWGKGTMVTVSS

SEQ ID NO: 27 - protein, 120 aa, synthetic construct
EVQLVQSGAEVKKPGSSVKVSCKASGDAFKSKTFTISWVRQAPGQGLEWLGGIIPVFGTITYAQKFQGRVTITADKSTNTAFMELSSLRSEDTAIYYCTRRGKWNPFDPWGPGTLVTVSS

SEQ ID NO: 28 - protein, 118 aa, synthetic construct
EVQLVQSGAEVKKPGSSVKVSCKASGGTFRSFGISWVRQAPGQGLEWMGGFIPVLGTANYAQKFQGRVTIIADKSTNTAYMELSSLRSEDTAVYYCARRGNWNPFDPWGQGTLVTVSS

SEQ ID NO: 29 - protein, 330 aa, synthetic construct [sequence omitted]

SEQ ID NO: 30 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 107 aa, synthetic construct [sequence omitted]

SEQ ID NO: 32 - protein, 458 aa, synthetic construct
QITLKESGPTLVKPTQTLTLTCTFSGFSLSTSGEGVGWIRQPPGKALEWLALIYWTDDKRYSPSLKSRLTITKDTSKNHVVLTMTNMDPVDTATYYCAHRRGGYSDHDSYYYYYLDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 33 - protein, 450 aa, synthetic construct
EVQLVQSGAEVKKPGSSVKVSCKASGDAFKSKTFTISWVRQAPGQGLEWLGGIIPVFGTITYAQKFQGRVTITADKSTNTAFMELSSLRSEDTAIYYCTRRGKWNPFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 458 aa, synthetic construct
QITLKESGPTLVKPTQTLTLTCTFSGFSLSTSGEGVGWIRQPPGKALEWLALIYWSDDKRYSPSLKNRLTITKDTSKNQVVLTMTNMDPVDTATYYCAHRRGGYSDHDSYYYYYMDVWGKGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 456 aa, synthetic construct
QITLKESGPTLVKPTQTLTLTCTFSGFSLSTSGEAVGWIRQPPGKALEWLALIYWSDDSRYSPSLKSRLTITRVTAKNQVVLKMTNMDPVDTATYYCAHRRGSYSGDYYYYYMDVWGKGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 458 aa, synthetic construct
RITLKESGPTLVKPTQTLTLTCTFSGFSLSTSGEGVGWIRQPPGKALEWLALIYWSDDKRYSPSLKNRLTITKDTSKNQVVLTMTNMDPVDTATYYCAHRRGGYSGYDSYYYYYMDVWGKGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 37 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 454 aa, synthetic construct
EVQLVESGGGLVQPGRSLRLSCATSGFTFDDYTMHWVRQAPGKGLEWVSDISWSSGSIGYADSVKGRFTISRDNAKNSLYLQMNSLRAEDTALYFCAKDHRGYGDYEGGGFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 448 aa, synthetic construct
EVQLVQSGAEVKKPGSSVKVSCKASGGTFRSFGISWVRQAPGQGLEWMGGFIPVLGTANYAQKFQGRVTIIADKSTNTAYMELSSLRSEDTAVYYCARRGNWNPFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 458 aa, synthetic construct
QITLKESGPTLVKPTQTLTLTCTFSGFSLSTSGEGVGWIRQPPGKALEWLALIYWTDDKRYSPSLKSRLTITKDTSKNHVVLTMTNMDPVDTATYYCAHRRGGYSDHDSYYYYYLDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 458 aa, synthetic construct
QITLKESGPTLVKPTQTLTLTCTFSGFSLSTSGEGVGWIRQPPGKALEWLALIYWSDDKRYSPSLKNRLTITKDTSKNQVVLTMTNMDPVDTATYYCAHRRGGYSDHDSYYYYYMDVWGKGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 456 aa, synthetic construct
QITLKESGPTLVKPTQTLTLTCTFSGFSLSTSGEAVGWIRQPPGKALEWLALIYWSDDSRYSPSLKSRLTITRVTAKNQVVLKMTNMDPVDTATYYCAHRRGSYSGDYYYYYMDVWGKGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 458 aa, synthetic construct
RITLKESGPTLVKPTQTLTLTCTFSGFSLSTSGEGVGWIRQPPGKALEWLALIYWSDDKRYSPSLKNRLTITKDTSKNQVVLTMTNMDPVDTATYYCAHRRGGYSGYDSYYYYYMDVWGKGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 45 - protein, 124 aa, synthetic construct
EVQLVESGGGLVQPGRSLRLSCATSGFTFDDYTMHWVRQAPGKGLEWVSDISWSSGSIGYADSVKGRFTISRDNAKNSLYLQMNSLRAEDTALYFCAKDHRGYGDYEGGGFDHWGQGTLVTVSS